SEQUENCE LISTING (69 sequences)

SEQ ID NO: 1 - RNA, 5446 nt, Homo sapiens
  source 1..5446: mol_type = genomic RNA
tactttgggaactggtgagtctccctgtccctagggctttttagtcacatgtccatccactgtttcaatgtaacatgcatctaggcaaggttaacgattaaatggttgggatgaaaggtcatcctttacggagaacatcagaatggtagataattcctgttccactttctttgatgaaacaagtaaagaagaaacaacacaatcatattaatagaagagtcttcgttccagacgcagtccaggaatcatgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggtgaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctagcagaaggccccatgggttagaaaaggactataagaataatttcttatttaattttattttttataaaatacagaatacatacaaaagtgtgtataaaatgtacgttttaaaaaaggataagtgaacacccatgaacctactacccaggttaagaaaataaatgtcaccaggtacttgagaaacccctcgattgtctacctcgatcgtacttccttgctacccacccctcccagggacaaccactgtcctgaatttcacgataattattcctttgcctttcatttctgttttatcacctttgtatgtatctttaaacaacatatacccttttttacttatgtaaatggactgactcatactgcatacatcttctatgacttgattcttttgttcaatattatatctgagattcatccatggtgatgcaaataggtgcattattttttttcactgctctgtagtctggcattgtatgaatacagcacaatgtatcagttttaatattggggatcattagcattattctcaggtttttaaaaattataagcagtactactatgg

SEQ ID NO: 2 - RNA, 1610 nt, Homo sapiens
  source 1..1610: mol_type = genomic RNA
acagtagctcacacctgtaatcccagcactttggaaggccgaggtgggcggatcacctgagctcaggagtttgagaccagcctgtctctactaacaatataaaaattagctgggagtcacggtgggcgcctgtaatcccagctactcgggaggctgaggcaggagaattgcttgaacccaggagacagaggttgtagtgagctgagatcgcaccactgcactctagccttggcaacagtgcaagactgtctcaaaaacagcaacagagagcaggacgtgagacttctacctgctcactcagaatcatttctgcaccaaccatggccacgtttgtggagctcagtaccaaagccaagatgcccattgtgggcctgggcacttggaagtctcctcttggcaaagtgaaagaagcagtgaaggtggccattgatgcaggatatcggcacattgactgtgcctatgtctatcagaatgaacatgaagtgggggaagccatccaagagaagatccaagagaaggctgtgaagcgggaggacctgttcatcgtcagcaagttgtggcccactttctttgagagaccccttgtgaggaaagcctttgagaagaccctcaaggacctgaagctgagctatctggacgtctatcttattcactggccacagggattcaagtctggggatgaccttttccccaaagatgataaaggtaatgccatcggtggaaaagcaacgttcttggatgcctgggaggccatggaggagctggtggatgaggggctggtgaaagcccttggggtctccaatttcagccacttccagatcgagaagctcttgaacaaacctggactgaaatataaaccagtgactaaccaggttgagtgtcacccatacctcacacaggagaaactgatccagtactgccactccaagggcatcaccgttacggcctacagccccctgggctctccggatagaccttgggccaagccagaagacccttccctgctggaggatcccaagattaaggagattgctgcaaagcacaaaaaaaccgcagcccaggttctgatccgtttccatatccagaggaatgtgattgtcatccccaagtctgtgacaccagcacgcattgttgagaacattcaggtctttgactttaaattgagtgatgaggagatggcaaccatactcagcttcaacagaaactggagggcctgtaacgtgttgcaatcctctcatttggaagactatcccttcaatgcagaatattgaggttgaatctcctggtgagattatacaggagattctctttcttcgctgaagtgtgactacctccactcatgtcccattttagccaagcttatttaagatcacagtgaacttagtcctgttatagacgagaatcgaggtgctgttttagacatttatttctgtatgttcaactaggatcagaatatcacagaaaagcatggcttgaataaggaaatgacaattttttccacttatctgatcagaacaaatgtttattaagcatcagaaactctgccaacactgaggatgtaaagatcaataaaaaaaataataatcataaccaacaaaaaaaaaaaaaaa

SEQ ID NO: 3 - RNA, 1625 nt, Homo sapiens
  source 1..1625: mol_type = genomic RNA
tccgggcttccccagacagacagctggcttacagggccacctgaagacgttccagggctccacaggccactgtcttctggaggggaacggatcgactgccggtgcgcccagccaaattcaactcctgagtcctcagtctctagtcccgggaaggtttcaccgagctgccctactccttgtaccccttctagctggccttagcatagctacgtcagcagctattggcacgactgccctgattcaaggagaaactggactaatatcactatctcaacaggatcgaggccatcaagctacagatggtcttacaaatggaaccccaagtgaactcaactaacaacttccaccaaggacccctggaccaacccgttggccctttgactggcctaaagagttcccttctgaaggacactacaagtgcagggccccttcttcgcccctatccagcatctcttctcggcaaagtgaaagaagcggtgaaggtggccattgatgcagaatatcgccacattgactgtgcctatttctatgagaatcaacatgaggtgggagaagccatccaagagaagatccaagagaaggctgtgatgcgggaggacctgttcatcgtcagcaaggtgtggcccactttctttgagagaccccttgtgaggaaagcctttgagaagaccctcaaggacctgaagctgagctatctggacgtctatcttattcactggccacagggattcaagactggggatgactttttccccaaagatgataaaggtaatatgatcagtggaaaaggaacgttcttggatgcctgggaggccatggaggagctggtggacgaggggctggtgaaagcccttggggtctcaaatttcaaccacttccagatcgagaggctcttgaacaaacctggactgaaatataaaccagtgactaaccaggttgagtgtcacccatacctcacgcaggagaaactgatccagtactgccactccaagggcatcaccgttacggcctacagccccctgggctctccggatagaccttgggccaaacctgaggacccttccctgctggaggatcccaagattaaggagattgctgcaaagcacaaaaaaaccacagcccaggttctgatccgtttccatatccagaggaatgtgacagtgatccccaagtctatgacaccagcacacattgttgagaacattcaggtctttgactttaaattgagtgatgaggagatggcaaccatactcagcttcaacagaaactggagggcctttgacttcaaggaattctctcatttggaggactttcccttcgatgcagaatattgaggttgaatctcctggtgagattacacaggggattctctttcttcgctgaagtgtgactgtctccactcaagaactattttagccaagcttatctgagatcacagtgaactttgtcctgttgtagaccagaatggaggtgctgttttagacatgtatttctgtatgttcaactaggataagaatatcacagaaaagcatggcctgaataagcaaatgacaattttttccacttatctgatctgatcaaatgtctgttaagcaccagaaactctgccaacactgaggatgtaa

SEQ ID NO: 4 - RNA, 1098 nt, Homo sapiens
  source 1..1098: mol_type = genomic RNA
gtaagaaacggttgaactggatgcaatttttatcacagcttgtgtaagactgcctctgtccctcctctcacatgccattggttaaccagcagacagtgtgctcaggggcgttgccagctcattgctcttatagcctgtgagggaggaagaaacatttgctaaccaggccagtgacagaaatggattcgaaataccagtgtgtgaagctgaatgatggtcacttcatgcctgtcctgggatttggcacctatgcgcctgcagaggttcctaaaagtaaagctctagaggccgtcaaattggcaatagaagccgggttccaccatattgattctgcacatgtttacaataatgaggagcaggttggactggccatccgaagcaagattgcagatggcagtgtgaagagagaagacatattctacacttcaaagctttggagcaattcccatcgaccagagttggtccgaccagccttggaaaggtcactgaaaaatcttcaattggactatgttgacctctatcttattcattttccagtgtctgtaaaggaggacatagggattttaacatggaagaagagccctaaacataactcctaattcctttctatggaacagaaagcaattttgaatccatacttccgtgattgcatgtctacaagaaaagagagtgcagaatcctcaaagcctctgcctcaaaaacttgaggaaatgacaatcatctccttgaaggcacaaggtcttatttatgattcctgatttcacctcttgggatgttcacagacacagagtttcatgaagctgtggtgtccagaaaacctgctgcacatagggtgcacaatgagtttccatcttcttgcctcttttcaaggggcaagaactcagtccgggaatgtcttaaactacaaaccttcatgggaaaccttgttgcttctgcttcctctcttttcacactggaggttttatttttgcttagccatgaattcttgtgtcattcataacttttgtcttaaggtactgaaaactagtcaggctagttaatgcaaaagggtatattagatatgataatgggaaatcaaagccagggctacattaagaa

SEQ ID NO: 5 - RNA, 1064 nt, Homo sapiens
  source 1..1064: mol_type = genomic RNA
gcccattgtttttgtaatctctgaggagaagcagcagcaaacatttgctagtcagacaagtgacagggaatggattccaaacaccagtgtgtaaagctaaatgatggccacttcatgcctgtattgggatttggcacctatgcacctccagaggttccgagaagtaaagctttggaggtcacaaaattagcaatagaagctgggttccgccatatagattctgctcatttatacaataatgaggagcaggttggactggccatccgaagcaagattgcagatggcagtgtgaagagagaagacatattctacacttcaaagctttggtccacttttcatcgaccagagttggtccgaccagccttggaaaactcactgaagaaagctcaattggactatgttgacctctatcttattcattctccaatgtctctaaaggtatgcagtttgtatgagcataaaattgcgcttctgctgtcattataaacattgtttatctggatagttgaacagagctttttattaggaggatgtagggattatcacacagaagaagaaccgtaagtggaacacctaatttcctttctttcgagtaaattttgaatcctacttctctaatgcacacctacaagagaagagagtacagcaacctcaaagcctcttcctcaaaaacttgaaattacaatagtctctttcaaggcactgtcttagttgtggcttttgagtccatctcttgggatgttcccagacacagagtttcatgcagttgtggtgcccaataaaactgctgcacatgtgatgcacaatgagtttccaccatctctccccatttcaagctgaagcagatttggtggaagccactatgcatggttcttaaattagaaacccttaatgtggacttgcaaagctttattattctgctgcctcttctttcacaatagagtttgaagctgtatttagccaggaattactgtgtagtgtataacttttgatttaaagttacagaaaactactcaggctagttaatgcaaaagagtttactgagttatgtaaaatgggaagtc

SEQ ID NO: 6 - RNA, 1192 nt, Homo sapiens
  source 1..1192: mol_type = genomic RNA
acaggatctgcttagtgaaagaagtggcaagcaatggatcccaaatatcagcgtgtagagctaaatgatggtcacttcatgcccgtattgggatttggcacctatgcacctccagaggttccgaggaacagagctgtagaggtcaccaaattagcaatagaagctggcttccgccatattgattctgcttatttatacaataatgaggagcaggttggactggccatccgaagcaagattgcagatggcagtgtgaagagagaagacatattctacacttcaaagctttggtgcactttctttcaaccacagatggtccaaccagccttggaaagctcactgaaaaaacttcaactggactatgttgacctctatcttcttcatttcccaatggctctcaagccaggtgagacgccactaccaaaagatgaaaatggaaaagtaatattcgacacagtggatctctctgccacatgggaggtcatggagaagtgtaaggatgcaggattggccaagtccatcggggtgtcaaacttcaactgcaggcagctggagatgatcctcaacaagccaggactcaagtacaagcctgtctgcaaccaggtagaatgtcatccttacctcaaccagagcaaactgctggatttctgcaagtcaaaagacattgttctggttgcccacagtgctctgggaacccaacgacataaactatgggtggacccaaactccccagttcttttggaggacccagttctttgtgccttagcaaagaaacacaaacgaaccccagccctgattgccctgcgctaccagctgcagcgtggggttgtggtcctggccaagagctacaatgagcagcggatcagagagaacatccaggtttttgaattccagttgacatcagaggatatgaaagttctagatggtctaaacagaaattatcgatatgttgtcatggattttcttatggaccatcctgattatccattttcagatgaatattagcatagagggtgttgcacgacatctagcagaaggccctgtgtgtggatggtgatgcagaggatgtctctatgctggtgactggacacacggcctctggttaaatccctcccctcctgcttggcaacttcagctagctagatatatccatggtccagaaagcaaacataataaatttttatcttgaagt

SEQ ID NO: 7 - RNA, 1425 nt, Homo sapiens
  source 1..1425: mol_type = genomic RNA
ggccccgcctccttgagtggtgcggagctttgtgatgcggagcttcgtgatgcacgccccgatgcctgcggggctataaaaacgctcgcaagcgccaagtctcctcaggagccgccggcaagggggcaacgaggaagctcttaagagcgcggccggaaagcagttgagttacagacatcctgccaaaatgatttcttcaaagcccagacttgtcgtaccctatggcctcaagactctgctcgagggaattagcagagctgttctcaaaaccaacccatcaaacatcaaccagtttgcagcagcttattttcaagaacttactatgtatagagggaatactactatggatataaaagatctggttaaacaatttcatcagattaaagtagagaaatggtcagaaggaacgacaccacagaagaaattagaatgtttaaaagaaccaggaaaaacatctgtagaatctaaagtacctacccagatggaaaaatctacagacacagacgaggacaatgtaaccagaacagaatatagtgacaaaaccacccagtttccatcagtttatgctgtgccaggcactgagcaaacggaagcagttggtggtctttcttccaaaccagccacccctaagactactaccccaccctcatcaccacctccaacagctgtctcaccagagtttgcctacgtcccagctgacccagctcagcttgctgctcagatgttagcaatggcaacaagtgaacgaggacaaccaccaccatgttctaacatgtggaccctttattgtctaactgataagaatcaacaaggtcacccatcaccgccacctgcacctgggccttttccccaagcaaccctctatttacctaatcctaaggatccacagtttcagcagcatccaccaaaagtcacttttccaacttatgtgatgggcgacaccaagaagaccagtgccccaccttttatcttagtaggctcaaatgttcaggaagcacagggatggaaacctcttcctggacatgctgtcgtttcacagtcagatgtcttgagatatgttgcaatgcaagtgcccattgctgttcctgcagatgagaaataccagaaacataccctaagtccccagaatgctaatcctccaagtggacaagatgtccccaggccaaaaagccctgttttcctttctgttgctttcccagtagaagatgtagctaaaaaaagttcaggatctggtgacaaatgtgctccctttggaagttacggtattgctggggaggtaaccgtgactactgctcacaaacgtcgcaaagcagaaactgaaaactgatccagaaatgacgctgtctgggtcaacatttcagggaggagtctgccaccagtgtaatgtatcaataaacttcatgcaagcataaaaaaaaaaaaaaa

SEQ ID NO: 8 - RNA, 1765 nt, Homo sapiens
  source 1..1765: mol_type = genomic RNA
atctgcctccagcactgcccatccttgcccctttccactgtccttggagcttcctgggcccttccctgggcctcaggatcccaccctccatcccgtctgccctgcaggatgccgcagctgagcctgtcctggctgggcctcgggcccgtggcagcatccccgtggctgcttctgctgctggttgggggctcctggctcctggcccgcgtcctggcctggacctacaccttctatgacaactgccgccgtctccagtgttttcctcaacccccgaaacagaactggttttggggacaccagggcctggtcactcccacggaagagggcatgaagacattgacccagctggtgaccacatatccccagggctttaagttgtggctgggtcctaccttccccctcctcattttatgccaccctgacatcatccggcctatcaccagtgcctcagctgctgtcgcacccaaggatatgattttctatggcttcctgaagccctggctgggggatgggctcctgctgagtggtggtgacaagtggagccgccaccgtcggatgttgacgcctgccttccatttcaacatcttgaagccttatatgaagattttcaacaagagtgtgaacatcatgcacgacaagtggcagcgcctggcctcagagggcagcgccagactggacatgtttgaacacatcagcctcatgaccttggacagtctgcagaaatgtgtcttcagctttgaaagcaattgtcaggagaagcccagtgaatatattgccgccatcttggagctcagtgcctttgtagaaaagagaaaccagcagattctcttgcacacggacttcctgtattatctcactcctgatgggcagcgcttccgcagggcctgccacctggtgcacgacttcacagatgccgtcatccaggagcggcgccgcaccctccccactcagggtattgatgatttcctcaagaacaaggcaaagtccaagactttagacttcattgatgtgcttctgctgagcaaggatgaagatgggaaggaattgtctgatgaggacataagagcagaagctgacaccttcatgtttgagggccatgacactacagccagtggtctctcctgggtcctataccaccttgcaaagcacccagaataccaggaacagtgccggcaagaagtgcaagagcttctgaaggaccgtgaacctatagagattgaatgggacgacctggcccagctgcccttcctgaccatgtgcattaaggagagcctgcggttgcatcccccagtcccggtcatctcccgatgttgcacgcaggactttgtgctcccagacggccgcgtcatccccaaaggcattgtctgccttatcaatattatcgggatccattacaacccaactgtgtggccagaccctgaggtctacgaccccttccgtttcaaccaagagaacatcaaggagaggtcacctctggcttttattcccttctcggcagggcccagaaactgcatcgggcaggcgttcgccatggctgagatgaaggtggtcctggcgctcacgttgctgcacttccgcatcctgccgacccacattgaaccccgcaggaaacccgagctgatattgcgcgcagagggtggactttggctgcgggtggagcccctgggtgcgaactcacagtgactgccctacccacccacccacctttgtagagtcccagaaacaaaactatgctgacaaaaaaaaaaaaaaaaaaaaaaaaaaa

SEQ ID NO: 9 - RNA, 7277 nt, Homo sapiens
  source 1..7277: mol_type = genomic RNA
aggtcagggggctggggacgcgcgtggggatcgctacccggctcggccactgctgggcggacacctgggcgcgccgccgcgggaggagcccggactcgggccgaggctgcccaggcaatgcgttcactcggcgcaaacatggctgcggccctgcgcgccgcgggcgtcctgctccgcgatccgctggcatccagcagctggagggtctgtcagccatggaggtggaagtcaggtgcagctgcagcggccgtcaccacagaaacagcccagcatgcccagggtgcaaaacctcaagttcaaccgcagaagaggaagccgaaaactggaatattaatgctaaacatgggaggccctgaaactcttggagatgttcacgacttccttctgagactcttcttggaccgagacctcatgacacttcctattcagaataagctggcaccattcatcgccaaacgccgaacccccaagattcaagagcagtaccgcaggattggaggcggatcccccatcaagatatggacttccaagcagggagagggcatggtgaagctgctggatgaattgtcccccaacacagcccctcacaaatactatattggatttcggtacgtccatcctttaacagaagaagcaattgaagagatggagagagatggcctagaaagggctattgctttcacacagtatccacagtacagctgctccaccacaggcagcagcttaaatgccatttacagatactataatcaagtgggacggaagcccacgatgaagtggagcactattgacaggtggcccacacatcacctcctcatccagtgctttgcagatcatattctaaaggaactggaccattttccacttgagaagagaagcgaggtggtcattctgttttctgctcactcactgcccatgtctgtggtcaacagaggcgacccatatcctcaggaggtaagcgccactgtccaaaaagtcatggaaaggctggagtactgcaacccctaccgactggtgtggcaatccaaggttggtccaatgccctggttgggtcctcaaacagacgaatctatcaaagggctttgtgagagggggaggaagaatatcctcttggttccgatagcatttaccagtgaccatattgaaacgctgtatgagctggacatcgagtactctcaagttttagccaaggagtgtggagttgaaaacatcagaagagctgagtctcttaatggaaatccattgttctctaaggccctggccgacttggtgcattcacacatccagtcaaacgagctgtgttccaagcagctgaccctgagctgtccgctctgtgtcaatcctgtctgcagggagactaaatccttcttcaccagccagcagctgtgacccccgccggtggaccccgtggcgttaggcaaatgcccaacctccagatacctccgatgtggagagggtgttatttagagatcaaggaaggaagtcatccttccttgatatatatacagcctttgggtacaaattgtgtggtttcttgaggattggactcttgatggatttctatttttatataactatacagtaagcatttgtattttctctctctaggtataagttactagtttggaatgtccatcaggacctttaataaatgagttaaaaatttgtcttatgagacacacctatttaagtacagattttggctttattgcccaaaaccctcctgaaagggtacggagagtcccctctgtgggctggcagtgtgaatgagatctgtttagtctcgtgcatatagttgctgttttttaaatgaacacagttgagtatttgaagtgaatttgaaaaagaaatgttacttaatctttccctaagcccatgggttacagaatgctagggaggcaatttggttacctgcaatggctgcttttgccagcgaggccaccattcattggtcatcttggtatttgtgtgtgaatctcactttcctcaatgtaaaaaggaatcaagtatggatttcagaggtgctcttagattccccatacacccaagggtaataaacgtgtacaagtacagtgttcatgatacgtgccttggtgggagtccgtggtgccacagggaaggggctcccactgcttctggtctccagggacagtgctgctggaaaggctagtgatgagcttcaccctggagctcctcccgggaccttgcaagcctctccatccagcatcttctctatcttagttgaatgccttctttctgaacatttgttttaagaattattttataaagtcaacaatactttgcttgaattctttcttaatttacgattttttattataaaaatgtatagtgatacaatgggacatgtgaagaatacagaaaagtaaccactttaatgcaataactgttatcataatattgtatttcgtggtagtcctttcctgtagatatttttaatgccatttaatgccattgtcaccttggatttatgagtgaaaagtgtttctaaaaatatagaaataatgtcagatcagagtctgatcttctatgtttgtatttaaatggattaaaagatccccggtggttccatgaagaatttgtaaagatcactttctctttcctccaagccctgaaactttgttcttcaaaagagcgtttctttttttttttttttttagccagtttataaagtggaagtattaggagattcataaatcttctatattgagaattggctatgttaataaatattacaacatcattaaggttttagctaagtttgattcatgctgtctgttaaatcaaaactgatctaaatcagaattattaaatgtgaggagcttttttaatacaggaaaagaaacatgtcatccacttgagttaatagttttcctacgttgatgacagccctcatgagtagcatccacatttttaaaatttcaaattggtttttctactagtagattgtgtttctagagaaagatacaaggcataggtgattgtttaggattttcctctagcctttgccattacctttttggggatgaggttcacagtagactttgagtgaccgtcccaccgtgaagtgaattctctgagctggtggtgtggtgctggaaggaaggttatttttggagccactctctccccttaaggatatttcccaagggcctgcttcaattctttgatgactttagaggtgaaaaaatatttttatggagatgatgcagaaaactccaattcaggagcccttgcgagtatatctgaagcacttatttgctaaggaaacctgaattgatagcagtactgtgctgtctggaataatgtccttgatactgagttgggaccagactggcttttatagtgacaggcaaagaggaatttattgagatcactgctcatggcatttgttgctgtaagaagtgttgcctttgattgttactaaccacggatgggtaacggtcatacattaggctagtgtttggtaggacaaaatctttttagagctttgagaattgtcatcctgttggtcaactttgaaatacaaatgtttgccctggtaattagcaatgaactgctggcagtttcttcagctgtgtatatacggatctggcttttaattgatgaatcaacttctacagaaacttttgcagggacagtgttgatgaggcagtttagcttgccagggtgatgataaagcccaggtccctgcatgtatagtgctcttctaaagaatatgcattcttgaactacttaactttttaaaaatcacaataaatttttgcactcaaaatttgcttcgtatcaggagaaatgaactcattgttttgttttgtttttttttttttttaagatggagtcttgctatgtcacccaggctggagggcagtggtgcgatctcggctcactgctacttccacctcctgggctcaagtgatcctcccacctcagcctccaagtagctgggactacaggagtgcttcaccacgctgggctacttttttatattttttgtagagatggggttttgccatgttgtccaggctggtcttgaactcctgggctcaagggattctcctgcctcagtctcccaaagtgctgggattacaaggatgagcctctgcacctggccctgaactcattattaaaagccctttaaatgtgaggctgggtgccgtgccttacatgtgtaattccaatactttggaaggccaaggttggaggattgcttgatcccaagagttcaagaccagcctgggcaacatagggagaccctgactctacaaaaaataaagtaaaaattaactgggtgtagtgtcacatgcctgtagttccagctacttaggaggctgaggtggtaggattgcttgagcccagcagtttgaggttgcagtgaggtgtgattgcaccactgcactccagcctgggtgacagaggaagaccctgtcccaaaaccaaaaaaaagaaaagaaatacagagactgggtcatttacaaaggaaagaggtttaattgactcggttcggctttctgaggaagccttaggaaattgacaatcatggcagaaggggaagcagatgtcttacatggcagtgagtgagagcaagcaaaggggaagagcccccttataaaaccatcagatctcgtgagaactggctgtcacaagaacagcatgggggaactgtctccatgttccaatctccttccaccaggtccctccctcaacacgtggggattatggggattacaatttgaaatgagatttgggtggggaacagagccaaatcatatcattccaccctggcccctcccaaatcacatgtcctttttacatttcaaaaccaatcatgccttcacaacagtcctccagagtcttaactcattccagcattaacccaaaagtccaagttcaaagtctcatccaagacaaggcaagtcccttctgcctgtgagcctgtaacattaaaagcaagttagtgacttccaagatacaatgggagtacagacattggtaaatgttcccattccaaatgggagaaattggccaaaacacaggggctacaggccccatgcaccactgcactccactgtgcaagtctgaaacccggcagggcactccttaaattttttttttttttttttgagatggagtctcgctctgttgcccaagctggagtacagtggcacgatctcggctcactgcaacctccgcctcttgggttcaaaggatcatcctgcctcagcctccggagtagctgggctactcaggcgtgtgccaccatgcccggctaatttttgtatttttagtagagatggggcctgaccatgttggtcaggctggtctctaattcctgacctcgtgatccacccgcctcagcctctgaaagtgttgggattacaggcgtgagccaccatccccggcctactcaataaatcttaaagttccggaataatctcctttgactccatgtctcacctccaggtcacgctgatgcaagaggtgggctaatctttctagtaaattccatatttaattcaagaaaccataacttaaggcatgtaaaagagatcctttgctcaatgtgatgccattgtgcttatccaaagtatattattattacccacaaagggtgagagattaggctgcagccataccccaagtggagtgagcagcaagacctgccccctgctcagagtgtagatgactgggggcacctgcattcctaggggctctgccgtatgagctcctgtcgatgcggcaaaggaccaccttgcccaacgacagcgggaaggcagaatttaaagctggcagctgtaagcgaacgtctatgtgtgcgcacgggggcacgtgaaggcacaggtgcatcagccaagaacctccaattcacctcttaaccttctcacctcacctgaaaccccttctgccagaatcctgaaggtggcccaggaacagggctcctaacgttaggtggaaatgggaaattcattgagatgtcacaagctggaataagaaaattctgagctcacccggaaactaatgccctaaattaagattattcagcttctcaatttttaatagcaaaatggagacctgagtgtggataacttttagtatctgtgggggatcctggaaccaattccctgccaatatagaaggacaactgtctacagtacttgaagtattattaactacattcgccatgctgtatgttagatccccagaacatatttatcctgcatatctaaaattttgatcattttacaaactttctattttttttgtcaattttctccagctagacacttgtgcaatacggctattatctgatctttgccttaaatgttgtgcttcttttccatatgcacgtattttgcaaaatataaagtgtgtagagctatatagcactcagccaagtggtgggtacctgcaggtgcttcagagaagtaaattgatgctgctaatatttgttgaatggcacgaatatgatgagcaatagcaggtggtgcccttcagccagaccatcgctccgtgcgtctgatgcatcttgccaaagagtagttctgggaggtggttgcctctagagaacacattcctcctattctggggtcccgtgagagaaagaaatgcttttgcttttgatgtgggactcttactaagcctttcttcagagaaaaggaagtgaaaaatgcaccccatgataatcagtttcttacaacatactgtgatagtaccggcttcgttgtttttagctggaatcattagcttccatttttagaataacagctattggctaaattaggctacagtaggccattaagatggatgttggaattaaaaacatttttggaaaaaagcctgctttgagcctttgttataagcccttgggtagagatctgggtcctgtttctgatttcttgtgagccttcactctgacagttttgtttccagaaacacactcttagcctgctcctgaaatgggaacagacaggccaacttcccctctccagtctcccctgcgggtcaaagctttactttcctgtcatgttaagaaagaatagatttaaccttgataatccatgtagtattctgtatttttaccttttccttatctgaaaaaaagtgtatatatggcatggaattgattgcacaggcacatggcatgttggcttgtgaaccaattgttaaaatttcaagttaatcattaaaataatatctttcaaattaagttatattaaaaacaaaggtaacattctaaattcaaaaaaaaaaaaaaaaaa

SEQ ID NO: 10 - RNA, 889 nt, Homo sapiens
  source 1..889: mol_type = genomic RNA
gcagttcggcggtcccgcgggtctgtctcttgcttcaacagtgtttggacggaacagatccggggactctcttccagcctccgaccgccctccgatttcctctccgcttgcaacctccgggaccatcttctcggccatctcctgcttctgggacctgccagcaccgtttttgtggttagctccttcttgccaaccaaccatgagctcccagattcgtcagaattattccaccgacgtggaggcagccgtcaacagcctggtcaatttgtacctgcaggcctcctacacctacctctctctgggcttctatttcgaccgcgatgatgtggctctggaaggcgtgagccacttcttccgcgaattggccgaggagaagcgcgagggctacgagcgtctcctgaagatgcaaaaccagcgtggcggccgcgctctcttccaggacatcaagaagccagctgaagatgagtggggtaaaaccccagacgccatgaaagctgccatggccctggagaaaaagctgaaccaggcccttttggatcttcatgccctgggttctgcccgcacggacccccatctctgtgacttcctggagactcacttcctagatgaggaagtgaagcttatcaagaagatgggtgaccacctgaccaacctccacaggctgggtggcccggaggctgggctgggcgagtatctcttcgaaaggctcactctcaagcacgactaagagccttctgagcccagcgacttctgaagggccccttgcaaagtaatagggcttctgcctaagcctctccctccagccaataggcagctttcttaactatcctaacaagccttggaccaaatggaaataaagctttttgatgcaaaaaaaaaaaaaaaaaaa

SEQ ID NO: 11 - RNA, 5000 nt, Homo sapiens
  source 1..5000: mol_type = genomic RNA
acccgtcgccacgcccgccgcaggccaagggccagtcacttgcgggccggcgtcccgcagcccattcgcgccccgcccctgccccgccgcgggatgagtaacggttacgaagcactttctcggctacgatttctgcttagtcattgtcttccaggaaacagctccctcagtttggaatcagctctcccgctgcggccgcagtagccggagccggagccgcagccaccggtgccttcctttcccgccgccgcccagccgccgtccggcctccctcgggcccgagcgcagaccaggctccagccgcgcggcgccggcagcctcgcgctccctctcgggtctctctcgggcctcgggcaccgcgtcctgtggggcggccgcctgcctgcccgcccgcccgcagccccttcgctgcgcggcccctgggcggccgctgccatgggcaccgacagccgcgcggccaaggcgctcctggcgcgggcccgcaccctgcacctgcagacggggaacctgctgaactggggccgcctgcggaagaagtgcccgtccacgcacagcgaggaggagtttccagatgtcttggaatgcactgtatctcatgcagtagaaaagataaatcctgatgaaagagaagaaatgaaagtttctgcaaaactgttcattgtagaatcaaactcttcatcatcaactagaagtgcagttgacatggcctgttcagtccttggagttgcacagctggattctgtgatcattgcttcacctcctattgaagatggagttaatctttccttggagcatttacagccttactgggaggaattagaaaacttagttcagagcaaaaagattgttgccataggtacctctgatctagacaaaacacagttggaacagctgtatcagtgggcacaggtaaaaccaaatagtaaccaagttaatcttgcctcctgctgtgtgatgccaccagatttgactgcatttgctaaacaatttgacatacagctgttgactcacaatgatccaaaagaactgctttctgaagcaagtttccaagaagctcttcaggaaagcattcctgacattcaagcgcacgagtgggtgccgctgtggctactgcggtattcggtcattgtgaaaagtagaggaattatcaaatcaaaaggctacattttacaagctaaaagaaggggttcttaactgacttaggagcataacttacctgtaatttccttcaatatgagagaaaattgagatgtgtaaaaatctagttactgcctgtaaatggtgtcattgaggcagatattctttcgtcatatttgacagtatgttgtctgtcaagttttaaatacttatcttgcctccatatcaatccattctcatgaacctctgtattgctttccttaaactattgttttctaattgaaattgtctataaagaaaatacttgcaatatatttttcctttatttttatgactaatataaatcaagaaaatttgttgttagatatattttggcctaggtatcagggtaatgtatatacatattttttatttccaaaaaaaattcattaattgcttcttaactcttattataaccaagcaatttaattacaattgttaaaactgaaatactggaagaagatatttttcctgtcattgatgagatatatcagagtaactggagtagctgggatttactagtagtgtaaataaaattcactcttcaatacatgaatggaaacttaaatttttttttatgtgtccttgcttatagtttagctgtaataatttaaccttgtattcttgtgccatattctgtctttttattacttataaagacaaaccaaagtaaatctgaaaggagactagaagctttgaaattattgtttgggggttttataaaagcaactactgtcacctccatccagattcttttaaattattgatccatccatagtatatattgctactcattcaagaatcctcaataagtattgagtatttaccatatgttgggatactgtgggctctggagagaggagggggcaatagagctaggaattaagaatcagttgagtaaaatgtgtaatatttattccccattaataactgactaggaaggactaaaagccagaaaggggatgaaaaaaaaatccttaattcagggccgacattatctacttaaacaactttgagatatggtcttaattattttaaagcagaataatataattgaaagtttatagctaaaagagactatataggtcatttagtataattcttcattagtttacgaaccacaaaattgcaaataaataagctatgaactttgatgtacactataaatctccttaattctataaatttgtgtctgtaacctgaatagtttgaaaacttctttaaaaatctcttgtatttcatccgggcgcagtggctcacacctgtaatcccagcactttgggaggccgaggtgggcagatcacgaggtcaggagtttgagaccagcctgaccaacatggtaaaaccccatctctactaaaatacaaaaattggctgggcgtggtggcactcgcctgtaatctcagctacttgggaggctgaggcaggagaatcgcttgaacccgggaggcggaggttacagtgagccgagatcacatcactgcactccagcctgggcgacagagcgagactccatctcaaaaaaaaaaaaaaactcttgtatctcaatatttttaaaccacaggcctaaataaaactaattttgctcaagttttctcaacctagggaaaaagaactatggttccatattcaaaataaatattatagacccttttcctaagtaggattttgtggtttactgattgggtaatttgatcattaaaattatgtgaaatctgcccgggcacacctcatgcctgtaatcccagcactctgggaggccaaggcagatgatcacctgaggtcaggagttctagaccagcctggctaacatggtgaaaccctgtatctgctaaaaatacaaaaattagccaggcgtggtggcgggctcctgtaatcccagctactttggaggcgaggcacgagaatcgcttgaacctgggaggcggagtttgcagtgagccgagatcacgccattgcactccagcctgggcgacagagcgagactgcgtctcaaaaaaaaaaaaaaaagaaaaattatgtgaaatcatgtgatttgcctgggaaaacttgtttagatattgagctacttatgccttctagcctttatattaattgtatgtaatgttattaaatatatatatagttcatctttacatttggaaatgcccaacatttttttcatataagtccttaaacaagcgttcattttattttaaatctatacagtgaactggccaagatattttaagagggaactttaatatcccatttattgtttttataaccctggacttataaaaatgggttgtttgaagggttattttgaaagtgggggaaaaaaaaacttagttgctaatgtatctaaacttcagcagagctttttggtgatctcctacctgcaccctcaactcttgacaaagaagcaagactatagattcattttctgaaggggatcatgtatggaattttttgatgagtttttacttttacctctctactcttgattttctattattgaatactcttttaaaacactgatttttaaggctttatatatgttttccaggctgatgttcacatctttttttcatgaactatcagaatatagtgaacacttttcaaatatttaaggacttaatgtttaaaaagccataaaatagagagtggtaatactaccaaataattacttaaaactgaaagctaagttatcaatagtttatataagagatgttttctgaggagatgtgcatccagtgagaccaaggtagaaagtttatataattgttttttttccagtaaatatgaaaaaaaaagctgtagcttgtttattacatgtccaaaatacagtggagccttactttaacacaatgtactgtaacttggaatttgttctgttatgagtctatcttgaattcccatccatgaaactgtagtcaccaaaagcaacaagtattttcacatgatgtaaaagaccatactatgatggccattgctagaaattgaatcacaaataatagctaataatttttcatttttcaaaaaagatcatttggatagcagctatgtataaaatggaaaataaaaaattattctattttgcatgaatagttcagactttcccataccacagccaagcagtaactaaaattaggatcttaattttcaatgataaaaggtctaaggttcatttaattatgctcctttaacactgtctttctagatttttcacccagtattttcaaaatttgggaatgtaaacaattgatatatttattgtatgttggctagcagttcatccttctgcaaaatatgcattcagagaaatgtgaagcttgttttaatgaagacttaaaccatttgtgtcatttgtgttttcatattcaaatacaccaaattaaaattctgaacctatatttttcatcattaacttcctaatataccagaacatatacctttttcatgtaaagttggcaatgggatatggcagttttatttttgaaaaatatgtaacatgactttaatatttttatagttttcagaattagaaacataggaagggaaaatgttttaattagataagtcaactttttatgtgtctgtagtggtgtactataatagcaaattataaagcattattaaatgtttataataatttttaatattacctacattatgaatttaactaaaataaagtgtgagttgtatattttttaattgggttgtttcaatagctggaagcatcctgaagcattatattgatttttgaactatttgaactcaaactgagtatgatttgaaaataaattaataatttaaaaacatccaaaaaaaaaaaaaaaaaa

SEQ ID NO: 12 - RNA, 2928 nt, Homo sapiens
  source 1..2928: mol_type = genomic RNA
tcctcctgggtcttgcctagcggcgggcgcatgcttagtcaccgtgaggctgcgcttgcccggggcccgcgcccccctaccccggggaccgcccccgggccgcccgccccacttggcgcgccacttccgcgtgcatggccctgctgccccgagccctgagcgccggcgcgggaccgagctggcggcgggcggcgcgcgccttccgaggcttcctgctgcttctgcccgagcccgcggccctcacgcgcgccctctcccgtgccatggcctgcaggcaggagccgcagccgcagggcccgccgcccgctgctggcgccgtggcctcctatgactacctggtgatcgggggcggctcgggcgggctggccagcgcgcgcagggcggccgagctgggtgccagggccgccgtggtggagagccacaagctgggtggcacttgcgtgaatgttggatgtgtacccaaaaaggtaatgtggaacacagctgtccactctgaattcatgcatgatcatgctgattatggctttccaagttgtgagggtaaattcaattggcgtgttattaaggaaaagcgggatgcctatgtgagccgcctgaatgccatctatcaaaacaatctcaccaagtcccatatagaaatcatccgtggccatgcagccttcacgagtgatcccaagcccacaatagaggtcagtgggaaaaagtacaccgccccacacatcctgatcgccacaggtggtatgccctccacccctcatgagagccagatccccggtgccagcttaggaataaccagcgatggattttttcagctggaagaattgcccggccgcagcgtcattgttggtgcaggttacattgctgtggagatggcagggatcctgtcagccctgggttctaagacatcactgatgatacggcatgataaggggattcaaaccgatgacaagggtcatatcatcgtagacgaattccagaataccaacgtcaaaggcatctatgcagttggggatgtatgtggaaaagctcttcttactccagttgcaatagctgctggccgaaaacttgcccatcgactttttgaatataaggaagattccaaattagattataacaacatcccaactgtggtcttcagccacccccctattgggacagtgggactcacggaagatgaagccattcataaatatggaatagaaaatgtgaagacctattcaacgagctttaccccgatgtatcacgcagttaccaaaaggaaaacaaaatgtgtgatgaaaatggtctgtgctaacaaggaagaaaaggtggttgggatccatatgcagggacttgggtgtgatgaaatgctgcagggttttgctgttgcagtgaagatgggagcaacgaaggcagactttgacaacacagtcgccattcaccctacctcttcagaagagctggtcacacttcgttgagaaccaggagacacgtgtggcgggcagtgggacccatagatcttctgaaatgaaacaaataatcacattgacttactgtttgagttttatgtatttctttattttaatcaggatcttctgatagtggaaatttttagtacataatagaacttatttatggagttagaaatttgtagtgttatccaggattgattttcatttgatcacatctcacagtaattaatattttcaagttttttttttattaacagctctgtgctagttttttttttctgttttagcctcatcccaaatataaagctttgtgaagtacaattaacttaatgtacttgaatgaatagaacttgctactttttttttttttttttttgagacagagttttgctctcattgcccaggctggagtgcggtggtgctatttcagctcaccacaacctctgcctcctgggttcaagtgattctcctgccttagcctcccgaatagctggaattacaggcacgcaccaccatgcctgactaattttgtatttttagtagacatggggtttctccatgttggtcaggctggtctcaaactcccaccttcaggtgatccgcccacctcggcctcctgaggtgctgagattacaggcgtgagccactgtgccagcttgctaattttcacagaagttgatggcaattcttcacatgtaaacagtgccagtgcacagaacctttatatattttttgaagccagtactgtgctctgcatataacaaagctgcttcaaggatgagacctttttctaaaagcatgtaatgtgagaagccggcctgccttattttcttttttcttttttaatgattaaaaatagtttgtggcaaggcacggtggctcaggcctgtaattctagcactttgggaggccgaggcaggaggattacttgagcctacaagtttgaggccagcatgcacagcatagcaagactgcatctctacagagagtaaaaaaaattacccgagtgtggtgatgtgcatctgtaatctcagctacttgggaggctgaggtgagaggatcacttgagcttgggtgaggtgaggctgcagtgagtcctgatcatgctgctgcactcaatcttggacaacagagcaagaccctgtctcaaaaaaaaaaaaaaaaaatatatatatatatatatattatttttatgaggtgaagtgcatcaaacttgggaaagatttgaggaggctgggaacctcctggaaaaccactccttgaagaaagatatgagagacatttagaagtgattcctgctttcagaaggaggtggattcaaatacatcaaaagtcccttcctctgctaagtgtttatagttcaatgaataatttcaatatttgtatgtgttcttgtcattttatttttttctgaaaaacttccaaaaatttgaaaataaaattacagccttttcttcttataaaaaaaaaaaaaaaa

SEQ ID NO: 13 - RNA, 1774 nt, Homo sapiens
  source 1..1774: mol_type = genomic RNA
gcagttctttgaatttctcaccctaagatctggcctgtacattttcaaggaattcttgagaggttcttggagagattctgggagccaaacactccattgggatcctagctggaatataaagaatggcttatcagtggagaccatcgacagttgagaaaagaagaagcccaaaaagtacaagaatgaaaatcgagagtttttagagaacaacttgtaatggagccttcatctcttgagctgccggctgacacagtgcagcgcattgcggctgaactcaaatgccacccaacggatgagagggtggctctccacctagatgaggaagataagctgaggcacttcagggagtgcttttatattcccaaaatacaggatctgcctccagttgatttatcattagtgaataaagatgaaaatgccatctatttcttgggaaattctcttggccttcaaccaaaaatggttaaaacatatcttgaagaagaactagataagtgggccaaaatagcagcctatggtcatgaagtggggaagcgtccttggattacaggagatgagagtattgtaggccttatgaaggacattgtaggagccaatgagaaagaaatagccctaatgaatgctttgactgtaaatttacatcttctaatgttatcattttttaagcctacgccaaaacgatataaaattcttctagaagccaaagccttcccttctgatcattatgctattgagtcacaactacaacttcacggacttaacattgaagaaagtatgcggatgataaagccaagagagggggaagaaaccttaagaatagaggatatccttgaagtaattgagaaggaaggagactcaattgcagtgatcctgttcagtggggtgcatttttacactggacagcactttaatattcctgccatcacaaaagctggacaagcgaagggttgttatgttggctttgatctagcacatgcagttggaaatgttgaactctacttacatgactggggagttgattttgcctgctggtgttcctacaagtatttaaatgcaggagcaggaggaattgctggtgccttcattcatgaaaagcatgcccatacgattaaacctgcattagtgggatggtttggccatgaactcagcaccagatttaagatggataacaaactgcagttaatccctggggtctgtggattccgaatttcaaatcctcccattttgttggtctgttccttgcatgctagtttagagatctttaagcaagcgacaatgaaggcattgcggaaaaaatctgttttgctaactggctatctggaatacctgatcaagcataactatggcaaagataaagcagcaaccaagaaaccagttgtgaacataattactccgtctcatgtagaggagcgggggtgccagctaacaataacattttctgttccaaacaaagatgttttccaagaactagaaaaaagaggagtggtttgtgacaagcggaatccaaatggcattcgagtggctccagttcctctctataattctttccatgatgtttataaatttaccaatctgctcacttctatacttgactctgcagaaacaaaaaattagcagtgttttctagaacaacttaagcaaattatactgaaagctgctgtggttatttcagtattattcgatttttaattattgaaagtatgtcaccattgaccacatgtaactaacaataaataatataccttacagaaaatctgaaaaaaaaaaaaaaaaa

SEQ ID NO: 14 - RNA, 3519 nt, Homo sapiens
  source 1..3519: mol_type = genomic RNA
ctcacacgccggctcggatgatctcctgccatgactcagcgcttctcgcaggctgccctgctggggacaccggcttcgctcgggcccctcccgacgcgtccaccccctctcgccacccacgcccgcccccagccgctgggcctttcccagtgcggccgccgccgccacagctgcagtcagcaccgtcaccccagcagcatccgccgcctgcaccgcgcgtgcggcccgccccggcctgaccccgccgccgaacccggcgccagccatggagcccgaagccccccgtcgccgccacacccatcagcgcggctacctgctgacacggaaccctcacctcaacaaggacttggcctttaccctggaagagagacagcaattgaacattcatggattgttgccaccttccttcaacagtcaggagatccaggttcttagagtagtaaaaaatttcgagcatctgaactctgactttgacaggtatcttctcttaatggatctccaagatagaaatgaaaaactcttttatagagtgctgacatctgacattgagaaattcatgcctattgtttatactcccactgtgggtctggcttgccaacaatatagtttggtgtttcggaagccaagaggtctctttattactatccacgatcgagggcatattgcttcagttctcaatgcatggccagaagatgtcatcaaggccattgtggtgactgatggagagcgtattcttggcttgggagaccttggctgtaatggaatgggcatccctgtgggtaaattggctctatatacagcttgcggagggatgaatcctcaagaatgtctgcctgtcattctggatgtgggaaccgaaaatgaggagttacttaaagatccactctacattggactacggcagagaagagtaagaggttctgaatatgatgattttttggacgaattcatggaggcagtttcttccaagtatggcatgaattgccttattcagtttgaagattttgccaatgtgaatgcatttcgtctcctgaacaagtatcgaaaccagtattgcacattcaatgatgatattcaaggaacagcatctgttgcagttgcaggtctccttgcagctcttcgaataaccaagaacaaactgtctgatcaaacaatactattccaaggagctggagaggctgccctagggattgcacacctgattgtgatggccttggaaaaagaaggtttaccaaaagagaaagccatcaaaaagatatggctggttgattcaaaaggattaatagttaagggacgtgcttccttaacacaagagaaagagaagtttgcccatgaacatgaagaaatgaagaacctagaagccattgttcaagaaataaaaccaactgccctcataggagttgctgcaattggtggtgcattctcagaacaaattctcaaagatatggctgccttcaatgaacggcctattatttttgctttgagtaatccaactagcaaagcagaatgttctgcagagcagtgctacaaaataaccaagggacgtgcaatttttgccagtggcagtccttttgatccagtcactcttccaaatggacagaccctatatcctggccaaggcaacaattcctatgtgttccctggagttgctcttggtgttgtggcgtgtggattgaggcagatcacagataatattttcctcactactgctgaggttatagctcagcaagtgtcagataaacacttggaagagggtcggctttatcctcctttgaataccattagagatgtttctctgaaaattgcagaaaagattgtgaaagatgcataccaagaaaagacagccacagtttatcctgaaccgcaaaacaaagaagcatttgtccgctcccagatgtatagtactgattatgaccagattctacctgattgttattcttggcctgaagaggtgcagaaaatacagaccaaagttgaccagtaggataatagcaaacatttctaactctattaatgaggtctttaaacctttcataatttttaaaggttggaatcttttataatgattcataagacacttagattaagattttactttaacagtctaaaaattgatagaagaatatcgatataaattgggataaacatcacatgagacaattttgcttcactttgccttctggttatttatggtttctgtctgaattattctgcctacgttctctttaaaagctgttgtacgtactacggagaaactcatcatttttatacaggacactaatgggaagaccaaaattactaataaattgacataaccaacattaaaactcataattattttgttgaccattttgttaaaatctacttttcaaaaaaaaaaagctagaaatgaatctaggcgtaggtgaacttttgctaagcagaaataacactactttgttgcctagagaaagataacttctcaagtatttttattccagtcctagatcatatatgttcttttgtgcaacggaattctaacagttctaagagaaagatcactgctgtttacagcgccttgtgcagccttagattttaatattcttttgtcattgttacatctcatagagtaaagctcttattaccttgatcctgagtcagaaatcccacctgaaatcaccttttttcccccttgatcaaacatcccatccttcagctaccatactgttgctacagggattttgtggactgtggcccctgtcccgaggttggcaccttcagttcagcacagcctgagcagtgagaaggtctgaaaggagagtatatagttaagatccttgagaaagggctgcctgaggaactgacctcttaaagatctcaggatctttaagacaacaagttaggttcctactggagttacctgccagaatggcctcttaattaactcaggtaatgaagagctaactgtgttataatcatcttgcttttgcctgaatttggagaaagtattataattaagttcccagtatcagaaatgtccttacataagattaaaatatcttgatgactaataccattctatgagaaagagtagttatatgcccagactgtattaatttactttagaaactaatgtttgaagtaatggaaaaaattttaaattataaagctaaggtgcaataacatttgctacttatttatagaattatttgaagaattttgtttttgaagtaatgctttaaggagtataagatattcaagataaattatactataaaatgattttattgaaagttgaaggttacacaaattgttttaggtatgagcagaagaggttaaggtatttctaaaggtaacatatagtcaagagtttcctcaaaatagttatttggagaagaatcagaatgtctgtgtatttcttgtctgtttctatgttgtcttatagctctgactaaatgtgtttacctatgcaaaagatttattaaagcatagaaaaggtgaatgaataaaaatataaaataattgtcctttttcttaaaa

SEQ ID NO: 15 - RNA, 2917 nt, Homo sapiens
  source 1..2917: mol_type = genomic RNA
ggcccttccggggctgcgcggctcccccgcctcggtgccggcaaaaatgtgcctagtcacggggccgctctcgggggaactgaggtcgccttcgggctgggacccggagccccttcgccgcgccccaagacctccttgagtgcgggctgcgacgcgctcaccccgctgggccgtctgtgggcgcggctttgcgaagtcatccatctctcggatcactctctggcagccttgagctctcttgaaagcccagccccgggacgagggaggagcgccttaagtgcccagcgggctcagaagccccgacgtgtggcggctgagccgggccccgcgcactttctcggccggggaggggttcgggctcgggcacccggagttggcccctcgtaacgccgcgggaaagtgcgggcgagggcagtggactctgaggccggagtcggcggcacccggggcttctagttcggacgcggtgccccctggtggcgctcaccgcgcgcgtggccttggcttccgtgacagcgctcggttggccgtcacagcagccctcggttggccctttcctgctttatagcgtgcaaacctcgccgcgccagggccaagggacaggttggagctgttgatctgttgcgcaattgctattttccccagagcggctttgtctttggatttagcgtttcagaattgcaattccaaaatgtgtaagacgggatattctcttctgtgctgtcaagggacatggatttgattgacatactttggaggcaagatatagatcttggagtaagtcgagaagtatttgacttcagtcagcgacggaaagagtatgagctggaaaaacagaaaaaacttgaaaaggaaagacaagaacaactccaaaaggagcaagagaaagcctttttcgctcagttacaactagatgaagagacaggttgcccacattcccaaatcagatgctttgtactttgatgactgcatgcagcttttggcgcagacattcccgtttgtagatgacaatgaggtttcttcggctacgtttcagtcacttgttcctgatattcccggtcacatcgagagcccagtcttcattgctactaatcaggctcagtcacctgaaacttctgttgctcaggtagcccctgttgatttagacggtatgcaacaggacattgagcaagtttgggaggagctattatccattcctgagttacagtgtcttaatattgaaaatgacaagctggttgagactaccatggttccaagtccagaagccaaactgacagaagttgacaattatcatttttactcatctataccctcaatggaaaaagaagtaggtaactgtagtccacattttcttaatgcttttgaggattccttcagcagcatcctctccacagaagaccccaaccagttgacagtgaactcattaaattcagatgccacagtcaacacagattttggtgatgaattttattctgctttcatagctgagcccagtatcagcaacagcatgccctcacctgctactttaagccattcactctctgaacttctaaatgggcccattgatgtttctgatctatcactttgcaaagctttcaaccaaaaccaccctgaaagcacagcagaattcaatgattctgactccggcatttcactaaacacaagtcccagtgtggcatcaccagaacactcagtggaatcttccagctatggagacacactacttggcctcagtgattctgaagtggaagagctagatagtgcccctggaagtgtcaaacagaatggtcctaaaacaccagtacattcttctggggatatggtacaacccttgtcaccatctcaggggcagagcactcacgtgcatgatgcccaatgtgagaacacaccagagaaagaattgcctgtaagtcctggtcatcggaaaaccccattcacaaaagacaaacattcaagccgcttggaggctcatctcacaagagatgaacttagggcaaaagctctccatatcccattccctgtagaaaaaatcattaacctccctgttgttgacttcaacgaaatgatgtccaaagagcagttcaatgaagctcaacttgcattaattcgggatatacgtaggaggggtaagaataaagtggctgctcagaattgcagaaaaagaaaactggaaaatatagtagaactagagcaagatttagatcatttgaaagatgaaaaagaaaaattgctcaaagaaaaaggagaaaatgacaaaagccttcacctactgaaaaaacaactcagcaccttatatctcgaagttttcagcatgctacgtgatgaagatggaaaaccttattctcctagtgaatactccctgcagcaaacaagagatggcaatgttttccttgttcccaaaagtaagaagccagatgttaagaaaaactagatttaggaggatttgaccttttctgagctagtttttttgtactattatactaaaagctcctactgtgatgtgaaatgctcatactttataagtaattctatgcaaaatcatagccaaaactagtatagaaaataatacgaaactttaaaaagcattggagtgtcagtatgttgaatcagtagtttcactttaactgtaaacaatttcttaggacaccatttgggctagtttctgtgtaagtgtaaatactacaaaaacttatttatactgttcttatgtcatttgttatattcatagatttatatgatgatatgacatctggctaaaaagaaattattgcaaaactaaccactatgtacttttttataaatactgtatggacaaaaaatggcattttttatattaaattgtttagctctggcaaaaaaaaaaaattttaagagctggtactaataaaggattattatgactgttaaattattaaaa

SEQ ID NO: 16 - RNA, 2423 nt, Homo sapiens
  source 1..2423: mol_type = genomic RNA
atcctccgcccagcaccccaggattcaggcgttgggtcccgcccttgtaggctgtccacctcaaacgggccggacaggatatataagagagaatgcaccgtgcactacacacgcgactcccacaaggttgcagccggagccgcccagctcaccgagagcctagttccggccagggtcgccccggcaaccacgagcccagccaatcagcgccccggactgcaccagagccatggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatgagattccttagcctggatttccttctaacatgttatcaaatctgggtatctttccaggcttccctgacttgctttagtttttaagatttgtgtttttctttttccacaaggaataaatgagagggaatcgactgtattcgtgcatttttggatcatttttaactgattcttatgattactatcatggcatataaccaaaatccgactgggctcaagaggccacttagggaaagatgtagaaagatgctagaaaaatgttctttaaaggcatctacacaatttaattcctctttttagggctaaagttttagggtacagtttggctaggtatcattcaactctccaatgttctattaatcacctctctgtagtttatggcagaagggaattgctcagagaaggaaaagactgaatctacctgccctaagggacttaacttgtttggtagttagccatctaatgcttgtttatgatatttcttgctttcaattacaaagcagttactaatatgcctagcacaagtaccactcttggtcagcttttgttgtttatatacagtacacagataccttgaaaggaagagctaataaatctcttctttgctgcagtcatctactttttttttaattaaaaaaaatttttttttgaagcagtcttgctctgttacccaggctggagtgcagtggtgtgatctcggctcactgcaacctctgcctcccaggttccagcaattctcctgcctcagcctccctagtagctgggatgacaggcgcctgccatcatgcctgactaatttttgtatttttagtagagacggcgtttcaccatgttggccaggctggtctcaaactcctgacctcaggtgatccgcctacctcagcctcccaaagtgctgggattacaggcgtgatccaccacacctggcccttgcaatcttctactttaaggtttgcagagataaaccaataaatccacaccgtacatctgcaatatgaattcaagaaaggaaatagtaccttcaatacttaaaaatagtcttccacaaaaaatactttatttctgatctatacaaattttcagaaggttattttctttatcattgctaaactgatgacttactatgggatggggtccagtcccatgaccttggggtacaattgtaaacctagagttttatcaactttggtgaacagttttggcataatagtcaatttctacttctggaagtcatctcattccactgttggtattatataattcaaggagaatatgataaaacactgccctcttgtggtgcattgaaagaagagatgagaaatgatgaaaaggttgcctgaaaaatgggagacagcctcttacttgccaagaaaatgaagggattggaccgagctggaaaacctcctttaccagatgctgactggcactggtggtttttgctctcgacagtatccacaatagctgacggctgggtgtttcagtttgaaaatattttgttgccttcatcttcactgcaattttgtgtaaatttctcaaagatctgaattaaataaataaaattcatttctacagacccacaaaaaaaaaaa

SEQ ID NO: 17 - RNA, 1591 nt, Homo sapiens
  source 1..1591: mol_type = genomic RNA
cgggcgccgcgggccatggcgggcgagaaccaccagtggcagggcagcatcctctacaacatgcttatgagcgcgaagcaaacgcgcgcggctcctgaggctccagagacgcggctggtggatcagtgctggggctgttcgtgcggcgatgagcccggggtgggcagagaggggctgctgggcgggcggaacgtggcgctcctgtaccgctgctgcttttgcggtaaagaccacccacggcagggcagcatcctctacagcatgctgacgagcgcaaagcaaacgtacgcggcaccgaaggcgcccgaggcgacgctgggtccgtgctggggctgttcgtgcggctctgatcccggggtgggcagagcggggcttccgggtgggcggcccgtggcactcctgtaccgctgctgcttttgtggtgaagaccacccgcggcagggcagcatcctctacagcttgctcactagctcaaagcaaacgcacgtggctccggcagcgcccgaggcacggccagggggcgcgtggtgggaccgctcctacttcgcgcagaggccagggggtaaagaggcgctaccaggcgggcgggccacggcgcttctgtaccgctgctgcttttgcggtgaagaccacccgcagcagggcagcaccctctactgcgtgcccacgagcacaaatcaagcgcaggcggctccggaggagcggccgagggccccctggtgggacacctcctctggtgcgctgcggccggtggcgctcaagagtccacaggtggtctgcgaggcagcctcagcgggcctgttgaagacgctgcgcttcgtcaagtacttgccctgcttccaggtgctgcccctggaccagcagctggtgctggtgcgcaactgctgggcgtccctgctcatgcttgagctggcccaggaccgcttgcagttcgagactgtggaagtctcggagcccagcatgctgcagaagatcctcaccaccaggcggcgggagaccgggggcaacgagccactgcccgtgcccacgctgcagcaccatttggcaccgccggcggaggccaggaaggtgccctccgcctcccaggtccaagccatcaagtgctttctttccaaatgctggagtctgaacatcagtaccaaggagtacgcctacctcaaggggaccgtgctctttaacccggacgtgccgggcctgcagtgcgtgaagtacattcagggactccagtggggaactcagcaaatactcagtgaacacaccaggatgacgcaccaagggccccatgacagattcatcgaacttaatagtacccttttcctgctgagattcatcaatgccaatgtcattgctgaactgttcttcaggcccatcatcggcacagtcagcatggatgatatgatgctggaaatgctctgtacaaagatataaagtcatgtgggccacacaagtgcagtagtgcagttcaccatgagggaagaataaagagctgtgggcaaaagagtgtaaaatattttaaaataaactttcttaatatttttacatgcagagtatttttgtattcaattaaagaaataattttattccaaaaaaa

SEQ ID NO: 18 - RNA, 1958 nt, Homo sapiens
  source 1..1958: mol_type = genomic RNA
acttccctctggcctctcagagcctcttggatccccacagggtaatgggtgtcccgatctcgcgggggactctgtgatccgtgttcccctgaccctcctagtgcacaacttggccgggctcactgggctcctgcaccactgcctgtcaggtccgctgccagccccaagccccccaccagccatgagctcctccagaaaggaccacctcggcgccagcagctcagagcccctcccggtcatcattgtgggtaacggcccctctggtatctgcctgtcctacctgctctccggctacacaccctacacgaagccagatgccatccacccacaccccctgctgcagaggaagctcaccgaggccccgggggtctccatcctggaccaggacctggactacctgtccgaaggcctcgaaggccgatcccaaagccccgtggccctgctctttgatgcccttctacgcccagacacagactttgggggaaacatgaagtcggtcctcacctggaagcaccggaaggagcacgccatcccccacgtggttctgggccggaacctccccgggggagcctggcactccatcgaaggctccatggtgatcctgagccaaggccagtggatggggctcccggacctggaggtcaaggactggatgcagaagaagcgaagaggtcttcgcaacagccgggccactgccggggacatcgcccactactacagggactacgtggtcaagaagggtctggggcataactttgtgtccggtgctgtagtcacagccgtggagtgggggacccccgatcccagcagctgtggggcccaggactccagccccctcttccaggtgagcggcttcctgaccaggaaccaggcccagcagcccttctcgctgtgggcccgcaacgtggtcctcgccacaggcacgttcgacagcccggcccggctgggcatccccggggaggccctgcccttcatccaccatgagctgtctgccctggaggccgccacaagggtgggtgcggtgaccccggcctcagaccctgtcctcatcattggcgcggggctgtcagcggccgacgcggtcctctacgcccgccactacaacatcccggtgatccatgccttccgccgggccgtggacgaccctggcctggtgttcaaccagctgcccaagatgctgtaccccgagtaccacaaggtgcaccagatgatgcgggagcagtccatcctgtcgcccagcccctatgagggttaccgcagcctccccaggcaccagctgctgtgcttcaaggaagactgccaggccgtgttccaggacctcgagggtgtcgagaaggtgtttggggtctccctggtgctggtcctcatcggctcccaccccgacctctccttcctgcctggggcaggggctgactttgcagtggatcctgaccagccgctgagcgccaagaggaaccccattgacgtggaccccttcacctaccagagcacccgccaggagggcctgtacgccatggggccgctggccggggacaacttcgtgaggtttgtgcaggggggcgccttggctgtggccagctccctgctaaggaaggagaccaggaagccaccctaacactcggccagacccgctggctcccaggccctgagaggacagagatgaccacatccctgctggatgcaggacccgtccaaagatgccccggggaggggtgtcagcccacgttgctggcctttggggtcaagaggagtagggatcccaggctgccctggacttagaccagtgtctgaggtggtaacagcggccgcaggccagggttggcctagacctgggatttgtggggaaagctgctggtgtgaccagctgagcacccagccaggagacctgcagccctgcgccttccagaagcaggtcccaaataaagccagtgcccacctgcaaaaaaaaaaaaaaaaaa

SEQ ID NO: 19 - RNA, 2432 nt, Homo sapiens
  source 1..2432: mol_type = genomic RNA
atggcccagtgagtgactcgccaggggcagcccggctcggcctcagcgggcggggaactctttgggggtcgagatctccctcgttctctccgacgcctcccaccctgggggtcgcctgagctcacttggggctctgtgaccctggccctacggcgtctcgggcccagagctccttccctgcgggcccggccccctgccctctcggccgcgcagagctgacatcgcgctgatcggattggccgtcatgggccagaacttaattctgaacatgaatgaccacggctttgtggtctgtgcttttaataggactgtctccaaagttgatgatttcttggccaatgaggcaaagggaaccaaagtggtgggtgcccagtccctgaaagagatggtctccaagctgaagaagccccggcggatcatcctcctggtgaaggctgggcaagctgtggatgatttcatcgagaaattggtaccattgttggatactggtgacatcatcattgacggaggaaattctgaatatagggacaccacaagacggtgccgagacctcaaggccaagggaattttatttgtggggagcggagtcagtggtggagaggaaggggcccggtatggcccatcgctcatgccaggagggaacaaagaagcgtggccccacatcaagaccatcttccaaggcattgctgcaaaagtgggaactggagaaccctgctgtgactgggtgggagatgagggagcaggccacttcgtgaagatggtgcacaacgggatagagtatggggacatgcagctgatctgtgaggcataccacctgatgaaagacgtgctgggcatggcgcaggacgagatggcccaggcctttgaggattggaataagacagagctagactcattcctgattgaaatcacagccaatattctcaagttccaagacaccgatggcaaacacctgctgccaaagatcagggacagcgcggggcagaagggcacagggaagtggaccgccatctccgccctggaatacggcgtacccgtcaccctcattggagaagctgtctttgctcggtgcttatcatctctgaaggatgagagaattcaagctagcaaaaagctgaagggtccccagaagttccagtttgatggtgataagaaatcattcctggaggacattcggaaggcactctacgcttccaagatcatctcttacgctcaaggctttatgctgctaaggcaggcagccaccgagtttggctggactctcaattatggtggcatcgccctgatgtggagagggggctgcatcattagaagtgtattcctaggaaagataaaggatgcatttgatcgaaacccggaacttcagaacctcctactggacgacttctttaagtcagctgttgaaaactgccaggactcctggcggcgggcagtcagcactggggtccaggctggcattcccatgccctgttttaccactgccctctccttctatgacgggtacagacatgagatgcttccagccagcctcatccaggctcagcgggattacttcggggctcacacctatgaactcttggccaaaccagggcagtttatccacaccaactggacaggccatggtggcaccgtgtcatcctcgtcatacaatgcctgatcatgctgctcctgtcaccctccacgattccacagaccaggacattccatgtgcctcatggcactgccacctggccctttgccctattttctgttcagttttttaaaagtgttgtaagagactcctgaggaagacacacagtttatttgtaaagtagctctgtgagagccaccatgccctctgcccttgcctcttgggactgaccaggagctgctcatgtgcgtgagagtgggaaccatctccttgcggcagtggcttccgcgtgccccgtgtgctggtgcggttcccatcacgcagacaggaagggtgtttgcgcactctgatcaactggaacctctgtatcatgcggctgaattccctttttcctttactcaataaaagctacatcagactgatgctctttctccagattcttagtctcacctcggccacatggagccattatccccattggcagaaagatttttctttaaaaaaaaagactagaataacacaagaaaccacatttaggattatgcttcactcagaggaggcaggcagggaggacacaccaggggctttaatacactgggcatgttttctttctccaattgggcaatgggtacatggacgttcactgtaacgtgctttttctttcgtcttttttttttttttttttttttttgctcctggcaagctgtgcgtgacattctttatggctttttgtatgtcaaatacttcatactaaactttctagagaattaaactttaatgatgggctcaaaaaaaaaaaaaa

SEQ ID NO: 20 - RNA, 4583 nt, Homo sapiens
  source 1..4583: mol_type = genomic RNA
gcggccgccccggcggctcctggaaccccggttcgcggcgatgccagccaccccagcgaagccgccgcagttcagtgcttggataatttgaaagtacaatagttggtttccctgtccacccgccccacttcgcttgccatcacagcacgcctatcggatgtgagaggagaagtcccgctgctcgggcactgtctatatacgcctaacacctacatatattttaaaaacattaaatataattaacaatcaaaagaaagaggagaaaggaagggaagcattactgggttactatgcacttgcgactgatttcttggctttttatcattttgaactttatggaatacatcggcagccaaaacgcctcccggggaaggcgccagcgaagaatgcatcctaacgttagtcaaggctgccaaggaggctgtgcaacatgctcagattacaatggatgtttgtcatgtaagcccagactattttttgctctggaaagaattggcatgaagcagattggagtatgtctctcttcatgtccaagtggatattatggaactcgatatccagatataaataagtgtacaaaatgcaaagctgactgtgatacctgtttcaacaaaaatttctgcacaaaatgtaaaagtggattttacttacaccttggaaagtgccttgacaattgcccagaagggttggaagccaacaaccatactatggagtgtgtcagtattgtgcactgtgaggtcagtgaatggaatccttggagtccatgcacgaagaagggaaaaacatgtggcttcaaaagagggactgaaacacgggtccgagaaataatacagcatccttcagcaaagggtaacctgtgtcccccaacaaatgagacaagaaagtgtacagtgcaaaggaagaagtgtcagaagggagaacgaggaaaaaaaggaagggagaggaaaagaaaaaaacctaataaaggagaaagtaaagaagcaatacctgacagcaaaagtctggaatccagcaaagaaatcccagagcaacgagaaaacaaacagcagcagaagaagcgaaaagtccaagataaacagaaatcggtatcagtcagcactgtacactagagggttccatgagattattgtagactcatgatgctgctatctcaaccagatgcccaggacaggtgctctagccattaggaccacaaatggacatgtcagttattgctctgtctaaacaacattcccagtagttgctatattcttcatacaagcatagttaacaacaaagagccaaaagatcaaagaagggatactttcagatggttgtcttgtgtgcttctctgcatttttaaaagacaagacattcttgtacatattatcaataggctataagatgtaacaacgaaatgatgacatctggagaagaaacatcttttccttataaaaatgtgttttcaagctgttgttttaagaagcaaaagatagttctgcaaattcaaagatacagtatcccttcaaaacaaataggagttcagggaagagaaacatccttcaaaggacagtgttgttttgaccgggagatctagagagtgctcagaattagggcctggcatttggaatcacaggatttatcatcacagaaacaactgttttaagattagttccatcactctcatcctgtatttttataagaaacacaagagtgcataccagaattgaatataccatatgggattggagaaagacaaatgtggaagaaatcatagagctggagactacttttgtgctttacaaaactgtgaaggattgtggtcacctggaacaggtctccaatctatgttagcactatgtggctcagcctctgttaccccttggattatatatcaacctgtaaacatgtgcctgtaacttacttccaaaaacaaaatcatacttattagaagaaaattctgattttatagaaaaaaaatagagcaaggagaatataacatgtttgcaaagtcatgtgttttctttctcaatgagggaaaaacaattttattacctgcttaatggtccacctggaactaaaagggatactattttctaacaaggtatatctagtaggggagaaagccaccacaataaatatatttgttaatagtttttcaagttttgttcactctgttttattgtttgttttattgagaaattcttactcttagagactcatgaattaagaaagagaattctgctaactcagagaacctggttcctatgtaattcagaatatattacatttctcagtaatatttgttttttgaatccacctttatctgagccaatggagatttacttatagcgtattaggagatatttattccattttcttattttaatcaacattctaattatagacacatgggcctccctagctgatttcactgctcccccttcattgcttagaaatgggcatcatttcttgtatgtcagatccccctgcatcttcaacatttagtcttttcttctccatattttctatctgtggatctctttaggggattgaagtcaccctagctgaaggcctcaccagtgtttcacagaggacacagcccaccccttgcaggaggaggtatctctgagtgtgcagcacagaatcgcatgacccaccttaaccttcctgttgtcatggaaggatgcacggctgctctgtccactgtgattcctagccctctcaagatcactgctttctgaagaatttgcaatgactctggcttctggctgcttatctctggacacccgttctccaccagttgtacagttcatgtaatctacttggcttaattgattttccacttctctcttcctcttctaagatataaacattttaaatgatttattcctgtttcttattctggtgtttctttccttgtccctatgagataagtgtctcaactcactaaatctattcccaatgtataaaataattctaattccattttcagctaaaacatatattaccaagaagaaacaaactttatcctacagaatgatgttaggtagaaatatgtccccaggtttgagacctttcggatgatttcatataccatctttcttctgagtgttacccagtcaagtataagtagccaaattatttttgcacatctttctgtttctcatgtcttcatttattcaacaagcacttactgggaaggtctacacctgcataggcaatgctggaaaaagggttaagtaaaccaggacatgacaatggtggcaaatgactatcaggtcttcccatgtgtttgactcaaacttattaccctatggtccttctgacaatggcagaaggtctgaatccttgatgctaaacttatataaaagtagaattattacaaaggaaaaagaaataaaaactaacattcattttcatatgttggatgaaatataaatgaagaaaaagataacatcaattttaactgtaattctccatccaccagtaacagatccttaagacaatagaatcatacagtattcaaaccagcagccttctcaaatttgagcaaaaactctatcaacctctggtaaagttcctacactagtcacagaaggtgttaactttctactctgattctgtctccataatggggtaaactgttgatagtttaccccatcaacagatggtcggtaaattattgattcgaagaatcgagagagtgcagcaacataaatctgttaatgtctgatcaagctcctgccctgttctccgaattcagcttcataattaagggaaggcctgttttctatcctcagatttaggttctagtagcagttgtgtaaccactagtgagtcacttaactcctctgggtccccatttctcatgtgcaacaagaaagaggggaactggagatgatcactctagttccagacaagggaacatttcacactttgtttacttcagggtgatgtccctgagtcctcattagtgactgcgtcctttggaagttatcccaaccctgcttttctcaaaagtgaaaatgtataggctctcagaggagacagatttaactctgcttctctaatgttattgaattaaaagctgttcacattagtggttattaaatattgaaataacactgggaagaaaaagcatatataaatacagctaaaaacaagaatagatattcattctcacaaagggagacagcaaagaaaatggaaagtgcactggtgctagcgttagacagcttgtgttaatgtctcaattctgctactaactggttgcagcttgtgtgaccttgggcacattgtatgatctcgcagaatatcatcccaaatctgcaaaatggaattggcatcatctcttttgcaagattgttatgagaattaaaaggttcttcattcaatataataataaatattttgtatataaatgaatatcaattaaaagttatgactaattccacaagtcaaacatataaattttatttcttgattcatgatatgtgatagtattcataaaaatgtacatgcatgataatttcaaggaataagtatatatgtgagaatcatggaaatgaaattaataatattaactagtaattaaattgtaa

SEQ ID NO: 21 - RNA, 9648 nt, Homo sapiens
  source 1..9648: mol_type = genomic RNA
ggtttgtaatgatagggcggcagcagcagcagcagcagcagtggtggaacgaggaggtggagaattgagagcacgatgcatacacaggtgtttctgagtagtaattagatcgctgtgaaggaaaaagcacacctttgagttttcacctgtgaacactatagcgctgagagagacagtctgaaagcagaggaagacatcgatcagtaacaccaagagacaccaaagttgaaagttttgttttctttccctctgttttatttttcccccgtgtgtccctactatggtcagaaagcctgttgtgtccaccatctccaaaggaggttacctgcagggaaatgttaacgggaggctgccttccctgggcaacaaggagccacctgggcaggagaaagtgcagctgaagaggaaagtcactttactgaggggagtctccattatcattggcaccatcattggagcaggaatcttcatctctcctaagggcgtgctccagaacacgggcagcgtgggcatgtctctgaccatctggacggtgtgtggggtcctgtcactatttggagctttgtcttatgctgaattgggaacaactataaagaaatctggaggtcattacacatatattttggaagtctttggtccattaccagcttttgtacgagtctgggtggaactcctcataatacgccctgcagctactgctgtgatatccctggcatttggacgctacattctggaaccattttttattcaatgtgaaatccctgaacttgcgatcaagctcattacagctgtgggcataactgtagtgatggtcctaaatagcatgagtgtcagctggagcgcccggatccagattttcttaaccttttgcaagctcacagcaattctgataattatagtccctggagttatgcagctaattaaaggtcaaacgcagaactttaaagacgccttttcaggaagagattcaagtattacgcggttgccactggctttttattatggaatgtatgcatatgctggctggttttacctcaactttgttactgaagaagtagaaaaccctgaaaaaaccattccccttgcaatatgtatatccatggccattgtcaccattggctatgtgctgacaaatgtggcctactttacgaccattaatgctgaggagctgctgctttcaaatgcagtggcagtgaccttttctgagcggctactgggaaatttctcattagcagttccgatctttgttgccctctcctgctttggctccatgaacggtggtgtgtttgctgtctccaggttattctatgttgcgtctcgagagggtcaccttccagaaatcctctccatgattcatgtccgcaagcacactcctctaccagctgttattgttttgcaccctttgacaatgataatgctcttctctggagacctcgacagtcttttgaatttcctcagttttgccaggtggctttttattgggctggcagttgctgggctgatttatcttcgatacaaatgcccagatatgcatcgtcctttcaaggtgccactgttcatcccagctttgttttccttcacatgcctcttcatggttgccctttccctctattcggacccatttagtacagggattggcttcgtcatcactctgactggagtccctgcgtattatctctttattatatgggacaagaaacccaggtggtttagaataatgtcagagaaaataaccagaacattacaaataatactggaagttgtaccagaagaagataagttatgaactaatggacttgagatcttggcaatctgcccaaggggagacacaaaatagggatttttacttcattttctgaaagtctagagaattacaactttggtgataaacaaaaggagtcagttatttttattcatatattttagcatattcgaactaatttctaagaaatttagttataactctatgtagttatagaaagtgaatatgcagttattctatgagtcgcacaattcttgagtctctgatacctacctattggggttaggagaaaagactagacaattactatgtggtcattctctacaacatatgttagcacggcaaagaaccttcaaattgaagactgagatttttctgtatatatgggttttgtaaagatggttttacacactatagatgtctatactgtgaaaagtgttttcaattctgaaaaaaagcatacatcatgattatggcaaagaggagagaaagaaatttattttacattgacattgcattgcttccccttagataccaatttagataacaaacactcatgctttaatggattatacccagagcactttgaacaaaggtcagtggggattgttgaatacattaaagaagagtttctaggggctactgtttatgagacacatccaggagttatgtttaagtaaaaatccttgagaatttattatgtcagatgttttttcattcattatcaggaagttttagttatctgtcatttttttttttcacatcagtttgatcaggaaagtgtataacacatcttagagcaagagttagtttggtattaaatcctcattagaacaaccacctgtttcactaataacttacccctgatgagtctatctaaacatatgcattttaagccttcaaattacattatcaacatgagagaaatcaccaacaaagaagatgttcaaaataatagtcccatatctgtaatcatatctacatgcaatgttagtaattctgaagttttttaaatttatggctatttttacacgatgatgaattttgacagtttgtgcattttctttatacattttatattcttctgttaaaatatctcttcagatgaaactgtccagattaattaggaaaaggcatatattaacataaaaattgcaaaagaaatgtcgctgtaaataagatttacaactgatgtttctagaaaatttccacttctatatctaggctttgtcagtaatttccacaccttaattatcattcaacttgcaaaagagacaactgataagaagaaaattgaaatgagaatctgtggataagtgtttgtgttcagaagatgttgttttgccagtattagaaaatactgtgagccgggcatggtggcttacatctgtaatcccagcactttgggaggctgagggggtggatcacctgaggtcgggagttctagaccagcctgaccaacatggagaaaccccatctctactaaaaatacaaaattagctgggcatggtggcacatgctggtaatctcagctattgaggaggctgaggcaggagaattgcttgaacccgggaggcggaggttgcagtgagccaagattgcaccactgtactccagcctgggtgacaaagtcagactccatctccaaaaaaaaaagattatatatatatatatatgtgtgtgtatgtgtgtgtgtgtgtgtgtgtgtatatatatatatatatatatacacacacacacacacactttttatatatatatatatatatatatagtggaacttacaaatgagagtaatataatgatgaaattttgaactgttatttataaacatctaaggtaaaatggttagtcatggccagagtatgtttcatcctttaatttttgtccatttgaaaataaggatttttgaaagaattataccaattaaaattattaaaggcaaacatagaattcataaaaaattgtccaaagtagaaatgatgacctataatttggagcatttccaattcagtaatttcaattttgctcttgaaaacatttaatatatatccaagactgacatttctttagctgaacctaacgtttgggtctctgagtgaatttataataactccttccttccttagcatagggttttcaaaatttgatttataattcctatttccagtaaatattgttcatttgtccacatctctccctatgatatgttgctggaggtaagaatttctttcatattcctattttttttttccccatagactaggctcatagaatttaaacaagcaaattttcctgagctttttcttgccaaatgaaagaagactggtaaattctcatagagaggtttgtgtagttcttggctcttcctggggttaatgtgcttatattcacagtggcaaattggtctcagactttaatttatttatttttgatttgaatttctctttaaaagtatcaatttaaaaggtaactagaattattctttctcattttcaaaagtgatttttgcattattaaatttccctgccattgtaatgccatttcacgcagaaaaaaagtcagccagtaattaagaaaaaaagtgatggagattaagtagtattttggcttatttttaggactcatcatgagaagacacagttcctttaatcaggaaattaatatccataattttcactcaaaattgcagtatgtaaagcagattctcaaaaactctcctgaacacttatttatatatatgtttttatataagtaaaatttttctcatatttttatacgatatgcacacacacacatacatgcacatactacttactacatgttctgtacttgtactttgtaccatgcatattcaaatgtttatatacataagtttattataacataaacagtaaaagtaatgaatactgtttaaaataactaatatagtattttttaatttttgtggggatggattctcaaatacttgtgattttaaaagattctaaagctaaaacacaacttgattttaaaaagaatgattctccttacacaattataaatatttgcagtaaatattttccttataatactgttttgaccccatttaaaaagtattagattatattcctttgatccaatgaaaactgaaccttataaatggttagctgaaagtagaccttattcttgtccttctttagaagagtaaagatttgtcctagggaagatggctgacttcggttcccaacatgcgtatgcatttagactgtagctcctcagccctgtggacacaaaatttggacagcttattaggttacgttagcaatgcatgacggtttctccaacactaagatattcacgttgaaacagatttcctgttcgtcttatgtgtctggtaaaattgtttccccaattacaatttgacatatcaatagagggttaacaagagtataattacataacagaattcctcatgaactgtaatcagtctacaggaaaatcattattttatcttgatttgcagatgaatatactgctaagaaagggagcaactctgacctttgttaaagttgatcttttgtaattgaggtataaggtatgaaaagataaaaaaccgaaggccagagaatcaggaaatgaaagatagtatggactgaaggtaacaatattttaatgttatgcaatatagtcagagaaatattaaaaattagttgtttgctgtgcataggtggatctcgcaggaagctaatgaaacctaagcttcagtgcctctcacttagacatgttccattcgaggtcctgaacctaactttgtattaggaattctgtactaattttgttgaagaagaccagcaaagttgtgtacacttctacccccacaaaatctgcattgtccatgtgagtaaagtaaaataattcctgttatttttttctgttagaaataagtatggaggatatgtttttaaaaatttatgagttaattgaaatatccatatataacaagtgactttctcacaatatatatgatgtgatatatagggagatagtttcactttcatcatattttatacgttgattctgaactatagaaaaataataaatgggattttaattatagctcttagttgggaaagaaatatagagagatgtgggatttgaatgcccatgaaagacattttattttacttgaatatattcttgcttcactttaccctccataatatgttgtacattagtgctgatcaagtttacagagttacattttgctttcctaaccattcagtcaggaattaaaatatggcattgtataacaactgggaagaagctcatagtggatataaattagagtagataatgggtcaccttgatagcctctgtttacattacttgtatatgggcaaaataattattacctatacgtgtatttaagcttaattttcatataaacagtatttttaatctatgttaaaatagataatatctaaaagtgtgatctctaggtagtccttagtttattagtactgtacttcaaaaagatttttaaataggtccggcacggtggctcatgcctgtaatcccagcactttgggaggctgaggcgggcgaatcacctgaggtcaggagttcgagatcagcctggccaacatggtgaaaccctgtctcaactaaaaatataaaaattagccgggcgtggtggcaggcgcctgtaatcccagctactcgggaggctgaggcaggagaatcacttgaacccaaggggcagaagctgcagttagccaagatcgcatcattgcactccagcctaggggacaagagcgcgagacttcatctcaaaaaaaaaaaaaaaaaaaaaaaaagatttttaaataatagctaaaggtatgctctctaggtcatccttagtttattagtactgtacttaaaaattattttttaatagtcaattttgggagataattatttctttccttatattttccaattagttggtgtctaaaaataaatgttttgtctaattttagatcaggtatacattcacaaaagcataaatcatagtctcacaggaaattcaccaattttccatatgtcgtgagataactgtcctttctacaacctcataacaatgaatttatataattacctagattttcttagtgtgaatctacccattagttttattttcttggtagttatttttttccctcctctctgttactattggccttaaaatacacagaggacggttacagtgtcctaatagctgttacatgtgtgtgtttcagcgtacttgaatcaagtgtacatttatagtaccaataaccgcctttacagctttacagttaacaattctctcacaaaactgtagagcattaggcatctgagagccatagagggccaactttgttccagagtgaacatgctttttttcctcaacatatacactactgattttttttaaaagtatgactttcaagtgaattaatgtattggttaggagaactgcttgctaagtccttattacctcttgttaaagcctcagaaggccgtgctgaaagccagaggggaaaaaaagagtaatgcacaggtatctcttttgcagtggtgactgtattttgagtaccttgtgtgacagggtattattacagcatcttgtgggaaaacctattaggcctttgcatgttaaagctgtataatttgttgggttgtgagtggtctgacttaaatgtgtattataaaatttagacatcaaattttcctactaactaactttattagatgcatacttggaagcacagtcatatcacactgggaggcaatgcaatgtggttacctggtcctaggtttgaactgtcttatttcaaaagatttctgaattaatttttccctagaatttctccttcattccaaagtacaaacatactttgaagaatgaaacagattgttcccatgaatgtatgctcatactcgactagaaacgatctatgttaaatgactgtgtatatgaattatttcaagtactaccccaaataactttcttattgctctgaaagaagaaaagcaatgtaaatcactatgattattgcacaaacaaccagaattctccaacaattttaagtaatctgatcctcttcttggagaaaattgttacctaatagtttttccttatgaatgttattactactggtataaatcaaatttctataaatttcctacttaagtcttaagaactgggttcttcctttgatgttattcatgttcagaaaggaaacaacactttactcttttaggacaattcctagaatctatagtagtatcaggatatattttgctttaaaatatattttggttattttgaatacagacattggctccaaattttcatctttgcacaatagtatgacttttcactagaacttctcaacatttgggaactttgcaaatatgagcatcatatgtgttaaggctgtatcatttaatgctatgagatacattgttttctccctatgccaaacaggtgaacaaacgtagttgttttttactgatactaaatgttggctacctgtgattttatagtatgcacatgtcagaaaaaggcaagacaaatggcctcttgtactgaatacttcggcaaacttattgggtcttcattttctgacagacaggatttgactcaatatttgtagagcttgcgtagaatggattacatggtagtgatgcactggtagaaatggtttttagttattgactcagaattcatctcaggatgaatcttttatgtctttttattgtaagcatatctgaatttactttataaagatggttttagaaagctttgtctaaaaatttggcctaggaatggtaacttcattttcagttgccaaggggtagaaaaataatatgtgtgttgttatgtttatgttaacatattattaggtactatctatgaatgtatttaaatatttttcatattctgtgacaagcatttataatttgcaacaagtggagtccatttagcccagtgggaaagtcttggaactcaggttacccttgaaggatatgctggcagccatctctttgatctgtgcttaaactgtaatttatagaccagctaaatccctaacttggatctggaatgcattagttatgaccttgtaccattcccagaatttcaggggcatcgtgggtttggtctagtgattgaaaacacaagaacagagagatccagctgaaaaagagtgatcctcaatatcctaactaactggtcctcaactcaagcagagtttcttcactctggcactgtgatcatgaaacttagtagaggggattgtgtgtattttatacaaatttaatacaatgtcttacattgataaaattcttaaagagcaaaactgcattttatttctgcatccacattccaatcatattagaactaagatatttatctatgaagatataaatggtgcagagagactttcatctgtggattgcgttgtttcttagggttcctagcactgatgcctgcacaagcatgtgatatgtgaaataaaatggattcttctatagctaaatgagttccctctggggagagttctggtactgcaatcacaatgccagatggtgtttatgggctatttgtgtaagtaagtggtaagatgctatgaagtaagtgtgtttgttttcatcttatggaaactcttgatgcatgtgcttttgtatggaataaattttggtgcaatatgatgtcattcaactttgcattgaattgaattttggttgtatttatatgtattatacctgtcacgcttctagttgcttcaaccattttataaccatttttgtacatattttacttgaaaatattttaaatggaaatttaaataaacatttgatagtttacataataa

SEQ ID NO: 22 - RNA, 2704 nt, Homo sapiens
  source 1..2704: mol_type = genomic RNA
cggcacctggcgagcggagccggagtcgggctggggaccgcggggtcgaggccggaccgcggcggggtcgggggagaaacgcgcgctgccctggcacgggcccccccccccggccgcgcggaatggtatggcccggccggagttaaggccggggggaggcggcgagtcccgcggcggcggcgacgatggggctgcgtgcaggaggaacgctgggcagggccggcgcgggtcggggggcgcccgaggggcccgggccgagcggcggcgcgcagggcggcagcatccactcgggccgcatcgccgcggtgcacaacgtgccgctgagcgtgctcatccggccgctgccgtccgtgttggaccccgccaaggtgcagagcctcgtggacacgatccgggaggacccagacagcgtgccccccatcgatgtcctctggatcaaaggggcccagggaggtgactacttctactcctttgggggctgccaccgctacgcggcctaccagcaactgcagcgagagaccatccccgccaagcttgtccagtccactctctcagacctaagggtgtacctgggagcatccacaccagacttgcagtagcagcctccttggcacctgctgccaccttcaagagcccagaagacacacctggcctccagcaggctgggccatgcagaagggatagcaggggtgcattctctttgcacctggcgagagggtctgactctgggcacccctctcaccagctacaaggccttggactcactgtacagtgtgggagccccagttcccacctctgtgacaataggatcatggccttacccttgaagcattaccgagaaggagaacagagatgggcttgaagagccacgtgctgccggctccaaattcccaaggacaaggatccctctgcatttttgtctatgtaacctcttatatggactacattcagctgcaaggaaaggaaaaccttgattgcagtggtttaaacaaacagaagattgtttttccacatagcatggattctggagatgggtggctaatggtattggttcaacaactccacgaaggtaggggtcacgtcttggatccttttgccttaatctcagtgctcgttacttcatggtcccaagatggctgctgtatccccaagaatcatgtctgcgttcaaggaaggaggggtggaggaagaggaagggccaaactagctggacccgtcaccttctatcagaaagtaaaacctcgtcagaagtctgtttcctgctctctccctctgcatatcttcacttagatgcccttggcccgagccagctaccattgcacctctagctgcaaacaaagctaagacagcagggaacagaattgtcatggctgaatagaccaatcgtgttccatctactgagactggcacactgcctcctgcaataaaactgggatcccattaccaagagagaaatgcagaattgtgtaccagttagcttttgctgtgtaacaaaccatccccaaacttggcagctagaaacaaaccctgtattttcccacaatcctatgggttggcaatttgggctgggctcaacagggcagttctgctgctcacacctgggatccctcatggagctaaggtcagctgttacctcagctgggcctggatggtctaggatagccttactcacttgcctggcaggtgacaggctgttggctggaattgcttggttctcctccatgtggcctctccagcaggctagctcaggcttattcacatgatggcttcaggattccaaagagagtgagagtagaagctgaaagacttcttgagttcttggcctggaactgggactaggacagtgtcacttctgctaagttcttttggtcagagcaaatcacaaggctttacccagattcaagggatgagaaacagactacatgtcttgatgaggggaaccacaaagagcttgtggccatttttcacctatcacaaataattttggatgggtatttatttggataaaggtatttccctcttccccctttctctctgtctcatggggcctcactctgccaagttggaaggcactaagacattgtcctggccctcagggtctaggggaagaggtgttggggcaggaagtgagtctctccatgggctggacccactgtagtaggagtgcctccttgtctgcactgctggtatggggttaggccaggtaggacattccagaggggcttctgaaaaccaagagtccctggggaaagggaacagagtaaggcaggccttgttctcactgccctctaagggaacttggtcactcggcacttttaagcctcagtttctccagttcaataataaggacaagagcttttcccatgcattctctttccccgggaaagttgactgaggtgaccagtaatagaattgaaaagggagagtgtcttcagtgcaatgtggcatcctggattgggtcttggaacaaaaacaggacattagtgggaaaattggaaatctgaaaaaagtctgaattttagttaatataccaatttcagtctcttggttttgacagatgtaccatggtgatgtaagatgttgaccttggggtaggctgggtgaagggtatacaggaactctttgtactatctctgcaacttctctgtaaatctagtatcattccaaaataaaagtttatttaatttaaaaaaaaaaaaaaaaa

SEQ ID NO: 23 - RNA, 1319 nt, Homo sapiens
  source 1..1319: mol_type = genomic RNA
cgcgcccgtcccgtcgccgccgccgccgccgcagacccctcggtcttgctatgtcgagctcacccgtgaagcgtcagaggatggagtccgcgctggaccagctcaagcagttcaccaccgtggtggccgacacgggcgacttccacgccatcgacgagtacaagccccaggatgctaccaccaacccgtccctgatcctggccgcagcacagatgcccgcttaccaggagctggtggaggaggcgattgcctatggccggaagctgggcgggtcacaagaggaccagattaaaaatgctattgataaactttttgtgttgtttggagcagaaatactaaagaagattccgggccgagtatccacagaagtagacgcaaggctctcctttgataaagatgcgatggtggccagagccaggcggctcatcgagctctacaaggaagctgggatcagcaaggaccgaattcttataaagctgtcatcaacctgggaaggaattcaggctggaaaggagctcgaggagcagcacggcatccactgcaacatgacgttactcttctccttcgcccaggctgtggcctgtgccgaggcgggtgtgaccctcatctccccatttgttgggcgcatccttgattggcatgtggcaaacaccgacaagaaatcctatgagcccctggaagaccctggggtaaagagtgtcactaaaatctacaactactacaagaagtttagctacaaaaccattgtcatgggcgcctccttccgcaacacgggcgagatcaaagcactggccggctgtgacttcctcaccatctcacccaagctcctgggagagctgctgcaggacaacgccaagctggtgcctgtgctctcagccaaggcggcccaagccagtgacctggaaaaaatccacctggatgagaagtctttccgttggttgcacaacgaggaccagatggctgtggagaagctctctgacgggatccgcaagtttgccgctgatgcagtgaagctggagcggatgctgacagaacgaatgttcaatgcagagaatggaaagtagcgcatccctgaggctggactccagatctgcaccgccggccagctgggatctgactgcacgtggcttctgatgaatcttgcgttttttacaaattggagcagggacagatcatagatttctgattttatgtaaaattttgcctaatacattaaagcagtcacttttcctgtgctgtttcaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - RNA, 2920 nt, Homo sapiens
  source 1..2920: mol_type = genomic RNA
gatttatggaaagcacctggtgcgtgttctgccctctgaggaccctgccctttcccaaaaggggtatcttaagaaggaaaatatggctgctctttgccggacagcagagtcccagtaggatttccattttccaaacctggtatcatctcctagttggaagaagtggtaagcccacgaacacaaatgcaggagggagaggtgccaagaagcagcggtacacagcaggcacagagacacgtggtcttcagcagagcctatggggttcagatgattcacataagaatagaagtttcagggctggacctggggaggcagcctgagcctgagccggctgtcctgagcctgagtactctagctgccttgtcgtcatcgcatctggctgccatccagcgccagcacacagtaatgagtggccgagcttcctctgggagggaggaaacagttaaaatcttgcagcagctgcaatcatctaggcgtggttctcttgtctgacttgggctgcacagatcctgggccaagggacagaagaaagacagcctaggagcagagcctcccagatggctgagttggatctaatggctccagggccactgcccagggccactgctcagcccccagcccctctcagcccagactctgggtcacccagcccagattctgggtcagccagcccagtggaagaagaggacgtgggctcctcggagaagcttggcagggagacggaggaacaggacagcgactctgcagagcagggggatcctgctggtgaggggaaagaggtcctgtgtgacttctgccttgatgacaccagaagagtgaaggcagtgaagtcctgtctaacctgcatggtgaattactgtgaagagcacttgcagccgcatcaggtgaacatcaaactgcaaagccacctgctgaccgagccagtgaaggaccacaactggcgatactgccctgcccaccacagcccactgtctgccttctgctgccctgatcagcagtgcatctgccaggactgttgccaggagcacagtggccacaccatagtctccctggatgcagcccgcagggacaaggaggctgaactccagtgcacccagttagacttggagcggaaactcaagttgaatgaaaatgccatctccaggctccaggctaaccaaaagtctgttctggtgtcggtgtcagaggtcaaagcggtggctgaaatgcagtttggggaactccttgctgctgtgaggaaggcccaggccaatgtgatgctcttcttagaggagaaggagcaagctgcgctgagccaggccaacggtatcaaggcccacctggagtacaggagtgccgagatggagaagagcaagcaggagctggagaggatggcggccatcagcaacactgtccagttcttggaggagtactgcaagtttaagaacactgaagacatcaccttccctagtgtttacgtagggctgaaggataaactctcgggcatccgcaaagttatcacggaatccactgtacacttaatccagttgctggagaactataagaaaaagctccaggagttttccaaggaagaggagtatgacatcagaactcaagtgtctgccgttgttcagcgcaaatattggacttccaaacctgagcccagcaccagggaacagttcctccaatatgcgtatgacatcacgtttgacccggacacagcacacaagtatctccggctgcaggaggagaaccgcaaggtcaccaacaccacgccctgggagcatccctacccggacctccccagcaggttcctgcactggcggcaggtgctgtcccagcagagtctgtacctgcacaggtactattttgaggtggagatcttcggggcaggcacctatgttggcctgacctgcaaaggcatcgaccggaaaggggaggagcgcaacagttgcatttccggaaacaacttctcctggagcctccaatggaacgggaaggagttcacggcctggtacagtgacatggagaccccactcaaagctggccctttccggaggctcggggtctatatcgacttcccgggagggatcctttccttctatggcgtagagtatgataccatgactctggttcacaagtttgcctgcaaattttcagaaccagtctatgctgccttctggctttccaagaaggaaaacgccatccggattgtagatctgggagaggaacccgagaagccagcaccgtccttggtggggactgctccctagactccaggagccatatcccagacctttgccagctacagtgatgggatttgcattttagggtgatttgggggcagaaataactgctgatggtagctggcttttgaaatcctatggggtctctgaatgaaaacattctccagctgctctcttttgctccatatggtgctgttctctatgtgtttgcagtaattcttttttttttttttgagacggagtctcgcactgttgcccaggctggagagcagtggcgcgatcttggctcactgcaagctccgcctcccgagttcaagcaattctcctgcctcagcctcccgagtagctgggattacaggtgcctgccaccacacccagctaatgttttgtatttttagtagagatggggtttcaccatgttggccaggcagatctcaaactcctgacctcgtgatgcacccacctcggcctcccaaagtgctgggattacatgcgtgagccactgcgccctgcctgtttgtagtaatttttaggcaccaaatctccctcatcttctagtgccattctcctctctgttcaggtaaatgtcacactgtgcccagaatggatgaccaggaaccttaaagagtggctgaaaagattgcagagttatcataataaattgctaacttgcgtaaaaaaaaaaaaaaaaaaaa

SEQ ID NO: 25 - RNA, 1982 nt, Homo sapiens
  source 1..1982: mol_type = genomic RNA
gtgcctcccggctggtttctgagatcagccacagaagttaaacttctttccagggaagaagggcggggatgtcagggctggagagtgcccgtgtccttctgtgtgcattgggctccttcctccttaattctctgctttccacttttaggctgaactccagtgcacccagttagacttggagcggaaactcaagttgaatgaaaatgccatctccaggctccaggctaaccaaaagtctgttctggtgtcggtgtcagaggtcaaagcagtggctgaaatgcagtttggggaactccttgctgctgtgaggaaggcccaggccaatgtgatgctcttcttagaggagaaggagcaagctgcgctgagccaggccaacggtatcaaggcccacctggagtacaggagtgccgagatggagaagagtaagcaggagctggagacgatggcggccatcagcaacactgtccagttcttggaggagtactgcaagtttaagaacactgaagacatcaccttccctagtgtttacatagggctgaaggataaactctcgggcatccgcaaagttatcacggaatccactgtacacttaatccagttgctggagaactataagaaaaagctccaggagttttccaaggaagaggagtatgacatcagaactcaagtgtctgccattgttcagcgcaaatattggacttccaaacctgagcccagcaccagggaacagttcctccaatatgtgcatgacatcacgttcgacccggacacagcacacaagtatctccggctgcaggaggagaaccgcaaggtcaccaacaccacgccctgggagcatccctacccggacctccccagcaggttcctgcactggcggcaggtgctgtcccagcagagtctgtacctgcacaggtactattttgaggtggagatcttcggggcaggcacctatgttggcctgacctgcaaaggcatcgaccagaaaggggaggagcgcagcagttgcatttccggaaacaacttctcctggagcctccaatggaacgggaaggagttcacggcctggtacagtgacatggagaccccactcaaagctggccctttctggaggctcggggtctatattgacttcccaggagggatcctttccttctatggcgtagagtatgattccatgactctggttcacaagtttgcctgcaagttttcagaaccagtctatgctgccttctggctttccaagaaggaaaacgccatccggattgtagatctgggagaggaacccgagaagccagcaccgtccttggtggggactgctccctagactccaggagccatatcccagacctttgccagctacagtgatgggatttgcattttagggtgatttgggggcaaaaataactgctgatggtagctggcttttgaaatcctatggggtctctgaatgaaaacattctccagctgctctcttttgctccatatggtgctgttctctatgtgtttgcagtaattctttttttttttttttttgagacggagtctcgcactgttgcccaggctggagtgcagtggcgtgatcttggctcactgcaagctccgcctcccgagttcaagcaattctcctgcctcagcctcccgagtagctgggattacaggtgcctgccaccacacccagctaacgttttgtatttttagtagagatggggtttcaccatgttggccaggcagatctcaaacttctgacctcgtgatgcactcacctcggcctcccaaagtgctgggattacaggcgtgagccactgcgccctgcctgtttgtagtaatttttaggcaccaaatctccctcatcttctagtgccattctcctctctgttcaggtaaatgtcacactgtgcccagaatggatgaccaggaaccttcaagagtggctgaaaagattgcagagttatcataataaattgctaacttgcgtatttcct

SEQ ID NO: 26 - RNA, 826 nt, Homo sapiens
  source 1..826: mol_type = genomic RNA
ctcgcaggctccaggggcggggcgtggccggggcgcagcgacgggcgcggaggtccggccgggcgcgcgcgcccccgccacacgcacgccgggcgtgccagtttataaagggagagagcaagcagcgagtcttgaagctctgtttggtgctttggatccatttccatcggtccttacagccgctcgtcagactccagcagccaagatggtgaagcagatcgagagcaagactgcttttcaggaagccttggacgctgcaggtgataaacttgtagtagttgacttctcagccacgtggtgtgggccttgcaaaatgatcaagcctttctttcatgatgttgcttcagagtgtgaagtcaaatgcatgccaacattccagttttttaagaagggacaaaaggtgggtgaattttctggagccaataaggaaaagcttgaagccaccattaatgaattagtctaatcatgttttctgaaaatataaccagccattggctatttaaaacttgtaatttttttaatttacaaaaatataaaatatgaagacataaacccagttgccatctgcgtgacaataaaacattaatgctaacactttttaaaaccgtctcatgtctgaatagctttcaaaataaatgtgaaatggtcatttaatgtattttcctatattctcaatcactttttagtaaccttgtaggccactgattattttaagattttaaaaattattattgctaccttaatgtattgctacaaaaatctcttgttgggggcaatgcaggtaataaagtagtatgttgttatttgtaaaaaaaaaaaaaaaaaa

SEQ ID NO: 27 - RNA, 3846 nt, Homo sapiens
  source 1..3846: mol_type = genomic RNA
agaccctcacgtgatgacaacagctagcaaagttctgtagctactgccttagggcatagtctaatttcttcagtaaaaacacacttattccaaatttggttccagaattgccttaaattgtttttgctctgttcttaggttgggggcggctatgagcaggcagaggatgtggtgtcacccaattaggagctctcagcttacgaggcaattagcataggttgccagggctgcacgaggagtggatttctgctttgtcattctgactctggcagttagcccgcccgctcggcgcagggcgtggcttctcgtagccattaggaaacagcaaccctttcacctcagttttcttcactccggcatttgcagcagagcgaaaggtggtcgagtcctgaaggagggcctgatgtcttcatcattctcaaattcttaggacggtcgggccctggaaggaacgctctcggaattggccgcggaaaccgatctgcccgttgtgtttgtgaaacagagaaagataggcggccatggtccaaccttgaaggaggcagcccaatatggcaagaaggtgatggtcctggactttgtcactcccacccctcttggaactagatggggtctcggaggaacatgtgtgaatgtgggttgcatacctaaaaaactgatgcatcaagcagctttgttaggacaagccctgcaagactctcgaaattatggatggaaagtcgaggagacagttaagcatgattgggacagaatgatagaagctgtacagaatcacattggctctttgaattggggctaccgagtagctctgcgggagaaaaaagtcgtctatgagaatgcttatgggcaatttattggtcctcacaggattaaggcaacaaataataaaggcaaagaaaaaatttattcagcagagagatttctcattgccactggtgaaagaccacgttacttgggcatccctggtgacaaagaatactgcatcagcagtgatgatcttttctccttgccttactgcccgggtaagaccctggttgttggagcatcctatgtcgctttggagtgcgctggatttcttgctggtattggtttagacgtcactgttatggttaggtccattcttcttagaggatttgaccaggacatggccaacaaaattggtgaacacatggaagaacatggcatcaagtttataagacagttcgtaccaattaaagttgaacaaattgaagcagggacaccaggccgactcagagtagtagctcagtccaccaatagtgaggaaatcattgaaggagaatataatacggtgatgctggcaataggaagagatgcttgcacaagaaaaattggcttagaaaccgtaggggtgaagataaatgaaaagactggaaaaatacctgtcacagatgaagaacagaccaatgtgccttacatctatgccattggcgatatattggaggataaggtggagctcaccccagttgcaatccaggcaggaagattgctggctcagaggctctatgcaggttccactgtcaagtgtgactatgaaaatgttccaaccactgtatttactcctttggaatatggtgcttgtggcctttctgaggagaaagctgtggagaagtttggggaagaaaatattgaggtttaccatagttacttttggccattggaatggacgattccgtcaagagataacaacaaatgttatgcaaaaataatctgtaatactaaagacaatgaacgtgttgtgggctttcacgtactgggtccaaatgctggagaagttacacaaggctttgcagctgcgctcaaatgtggactgaccaaaaagcagctggacagcacaattggaatccaccctgtctgtgcagaggtattcacaacattgtctgtgaccaagcgctctggggcaagcatcctccaggctggctgctgaggttaagccccagtgtggatgctgttgccaagactgcaaaccactggctcgtttccgtgcccaaatccaaggcgaagttttctagagggttcttgggctcttggcacctgcgtgtcctgtgcttaccaccgcccaaggcccccttggatctcttggataggagttggtgaatagaaggcaggcagcatcacactggggtcactgacagacttgaagctgacatttggcagggcatcgaagggatgcatccatgaagtcaccagtctcaagcccatgtggtaggcggtgatggaacaactgtcaaatcagttttagcatgacctttccttgtggattttcttattctcgttgtcaagttttctagggttgaatttttttcttttttctccatggtgttaatgatattagagatgaaaaacgttagcagttgatttttgtccaaaagcaagtcatggctagagtatccatgcaaggtgtcttgttgcatggaagggatagtttggctcccttggaggctatgtaggcttgtcccgggaaagagaactgtcctgcagctgaaatggactgttctttactgacctgctcagcagtttcttctctcatatattcccaaaacaagtacatctgcgatcaactctagccaaatttgcccctgtgtgctacatgatggatgattattattttaaggtctgtttaggaagggaaatggctacttggccagccattgcctggcatttggtagtatagtatgattctcaccattatttgtcatggaggcagacatacaccagaaatgggggagaaacagtacatatctttctgtctttagtttattgtgtgctggtctaagcaagctgagatcatttgcaatggaaaacacgtaacttgtttaaaagtttttctggtagctttagctttatgctaaaaaaaataatgacattgggtatctatttctttctaagactacattagtaggaaaataagtcttttcatgcttatgatttagctgttttgtggtaattgctttttaaaggaagttattaatatcataagttattattaatattttgaacacaggtggatgtgaaggattttcatttaaaaaccaagtggttttgactttttctgttgaatgaacaactgtgccttgtggaatttttgcagaagtgtttatgctttgttagcatttcaacttgcattattataaagaggtattaatgcctcagttatgtgtttgtcaatgtactggctgaggattctatctcagctgtcttttctaactgtgtaggttgagttttgaacacgtgcttgtggacatcaggcctcctgccagcagttcttgaagcttctttttcattcctgctactctacctgtatttctcagttgcagcactgagtggtcaaaatacatttctgggccacctcagggaacccatgcatctgcctggcatttaggcagcagagcccctgaccgtcccccacagggctctgcctcacgtcctcatctcatttggctgtgtaaagaaatgggaaaagggaaaaggagagagcaattgaggcagttgaccatattcagttttatttatttatttttaatttgtttttttctccaagtccaccagtctctgaaattagaacagtaggcggtatgagataatcaggcctaatcatgttgtgattctcttttcttagtggagtggaatgttctatccccacaagaaggattatatcttatagacttgtcttgttcagattctgtatttacccattttattgaaacatatactaagttccatgtatttttgttacaaatcttctgaaaaaaaacaaaacaatgtgaaacattaaaattaaaaggcattaataatatccacgtgtgccttcttactgaaaaaaaaaaa

SEQ ID NO: 28 - RNA, 3013 nt, Homo sapiens
  source 1..3013: mol_type = genomic RNA
gtgaaggaaatagggacctggccctgggccttgtgtagcgggagggggagctaggaagcagctgagggcagaatccaggagggcctggctgcgggggaatgaagcctccgccttcgcaggcaaaagcctttaaatacgggctcaggcccgggactcagagtgtaacgcgtggcagcctgagggaggggcgtgcgccgagagggagctcagatcgagcggggcgcgggtggagaagctgcggcggcgcggcccgtaggaaggtgctgtccgaacgatcgggataggagcggtccctgcgcttgctgctgggaagtggtacaatcatgtttgaaattaagaagatctgttgcatcggtgcaggctatgttggaggacccacatgtagtgtcattgctcatatgtgtcctgaaatcagggtaacggttgttgatgtcaatgaatcaagaatcaatgcgtggaattctcctacacttcctatttatgagccaggactaaaagaagtggtagaatcctgtcgaggaaaaaatctttttttttctaccaatattgatgatgccatcaaagaagctgatcttgtatttatttctgtgctgtccaaccctgagtttctggcagagggaacagccatcaaggacctaaagaacccagacagagtactgattggaggggatgaaactccagagggccagagagctgtgcaggccctgtgtgctgtatatgagcactgggttcccagagaaaagatcctcaccactaatacttggtcttcagagctttccaaactggcagcaaatgcttttcttgcccagagaataagcagcattaactccataagtgctctgtgtgaagcaacaggagctgatgtagaagaggtagcaacagcgattggaatggaccagagaattggaaacaagtttctaaaagccagtgttgggtttggtgggagctgtttccaaaaggatgttctgaatttggtttatctctgtgaggctctgaatttgccagaagtagctcgttattggcagcaggtcatagacatgaatgactaccagaggaggaggtttgcttcccggatcatagatagtctgtttaatacagtaactgataagaagatagctattttgggatttgcattcaaaaaggacactggtgatacaagagaatcttctagtatatatattagcaaatatttgatggatgaaggtgcacatctacatatatatgatccaaaagtacctagggaacaaatagttgtggatctttctcatccaggtgtttcagaggatgaccaagtgtcccggctcgtgaccatttccaaggatccatatgaagcatgtgatggtgcccatgctgttgttatttgcactgagtgggacatgtttaaggaattggattatgaacgcattcataaaaaaatgctaaagccagcctttatcttcgatggacggcgtgtcctggatgggctccacaatgaactacaaaccattggcttccagattgaaacaattggcaaaaaggtgtcttcaaagagaattccatatgctccttctggtgaaattccgaagtttagtcttcaagatccacctaacaagaaacctaaagtgtagagattgccatttttatttgtgattttttttttttttttttggtacttcaggatagcaaatatctatctgctattaaatggtaaatgaaccaagtgtttttttttgttttttttttgagacagagtctcactgttgcccaggctggagtgcagtggtgcaatctcggctcactgcaagctctgcttcccaggttcacgccattctcctggctcagcctcccaagtagctgggactacaggcacccgccacagtgcctggctaattttttgtatttttagtagagacagggtttcaccatgtgagccaggatggtctcaatctcctgaccttgtgaaccacccgtctcggcctcccaaagtgctgggattacaggtgtgagccaccacgcctggcccatgaaccaagtgtttttaaggaaacaaaactatttttttaatcatcagatttatactagctatatggatattagcatatctggtaattatgaatctagaatttttttacatatttttataatactgttagctcagttattggatgagtgaaagataatcatgttggttttaatagtgtcaatttttgtaaaataaaaattaaacttcaaactctttactttataaattgtccataggccacactttaatatcacattataaagggaaggacagtcttcattcctcctggttattggtttgtttgtcattaaagatatattttgaatccatgaaattgctatgctaaacagcctttacatgtatggtctggttaaagttcctttgttccttttgttttaataaaatgtgtcactgattttttagctcaaaatcatcactgttaatttccagtcaccccaaatatggttaaaagattttttttttaatcatgaagagaaaattagtagcatttctttctctccccattatttattggttttcctcactaatctttttttttttagtccaaaagccaaaaatatttatcttggttttacattttaatttccattcttaattgtaatttttttctttaaataaggaaaccaatataatctcatgtataaaaacttaaatattttacaagttacatatagcatcattctaaaataagaattttttttgttttctgtctgcttttttcttatgtctcttgttgagttttatattttcagtggttatttttgcttgtgttagatcattattaaaatatatccaatgtccctttgatacttgtgctctgctgagaatgtacagtttgcattaaacatcccaggtctcatccttcaggaattttgcagttcaatgagaagagggagacaaatataaagatgaggacagaagcatctctacagatgaaaattacataaataaaacattctccatcaacaactaaaaaaaaaaaaaaaaaaa

SEQ ID NO: 29 - RNA, 19 nt, Homo sapiens
  source 1..19: mol_type = genomic RNA
tggagtaagtcgagaagta

SEQ ID NO: 30 - RNA, 19 nt, Homo sapiens
  source 1..19: mol_type = genomic RNA
acaactagatgaagagaca

SEQ ID NO: 31 - RNA, 19 nt, Homo sapiens
  source 1..19: mol_type = genomic RNA
tgacagaagttgacaatta

SEQ ID NO: 32 - RNA, 19 nt, Homo sapiens
  source 1..19: mol_type = genomic RNA
gtaagaagccagatgttaa

SEQ ID NO: 33 - protein, 1545 aa, Homo sapiens
MLEKFCNSTFWNSSFLDSPEADLPLCFEQTVLVWIPLGYLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 34 - protein, 316 aa, Homo sapiens
MATFVELSTKAKMPIVGLGTWKSPLGKVKEAVKVAIDAGYRHIDCAYVYQNEHEVGEAIQEKIQEKAVKREDLFIVSKLWPTFFERPLVRKAFEKTLKDLKLSYLDVYLIHWPQGFKSGDDLFPKDDKGNAIGGKATFLDAWEAMEELVDEGLVKALGVSNFSHFQIEKLLNKPGLKYKPVTNQVECHPYLTQEKLIQYCHSKGITVTAYSPLGSPDRPWAKPEDPSLLEDPKIKEIAAKHKKTAAQVLIRFHIQRNVIVIPKSVTPARIVENIQVFDFKLSDEEMATILSFNRNWRACNVLQSSHLEDYPFNAEY

SEQ ID NO: 35 - protein, 316 aa, Homo sapiens
MATFVELSTKAKMPIVGLGTWRSLLGKVKEAVKVAIDAEYRHIDCAYFYENQHEVGEAIQEKIQEKAVMREDLFIVSKVWPTFFERPLVRKAFEKTLKDLKLSYLDVYLIHWPQGFKTGDDFFPKDDKGNMISGKGTFLDAWEAMEELVDEGLVKALGVSNFNHFQIERLLNKPGLKYKPVTNQVECHPYLTQEKLIQYCHSKGITVTAYSPLGSPDRPWAKPEDPSLLEDPKIKEIAAKHKKTTAQVLIRFHIQRNVTVIPKSMTPAHIVENIQVFDFKLSDEEMATILSFNRNWRAFDFKEFSHLEDFPFDAEY

SEQ ID NO: 36 - protein, 323 aa, Homo sapiens
MDSKYQCVKLNDGHFMPVLGFGTYAPAEVPKSKALEAVKLAIEAGFHHIDSAHVYNNEEQVGLAIRSKIADGSVKREDIFYTSKLWSNSHRPELVRPALERSLKNLQLDYVDLYLIHFPVSVKPGEEVIPKDENGKILFDTVDLCATWEAMEKCKDAGLAKSIGVSNFNHRLLEMILNKPGLKYKPVCNQVECHPYFNQRKLLDFCKSKDIVLVAYSALGSHREEPWVDPNSPVLLEDPVLCALAKKHKRTPALIALRYQLQRGVVVLAKSYNEQRIRQNVQVFEFQLTSEEMKAIDGLNRNVRYLTLDIFAGPPNYPFSDEY

SEQ ID NO: 37 - protein, 323 aa, Homo sapiens
MDSKHQCVKLNDGHFMPVLGFGTYAPPEVPRSKALEVTKLAIEAGFRHIDSAHLYNNEEQVGLAIRSKIADGSVKREDIFYTSKLWSTFHRPELVRPALENSLKKAQLDYVDLYLIHSPMSLKPGEELSPTDENGKVIFDIVDLCTTWEAMEKCKDAGLAKSIGVSNFNRRQLEMILNKPGLKYKPVCNQVECHPYFNRSKLLDFCKSKDIVLVAYSALGSQRDKRWVDPNSPVLLEDPVLCALAKKHKRTPALIALRYQLQRGVVVLAKSYNEQRIRQNVQVFEFQLTAEDMKAIDGLDRNLHYFNSDSFASHPNYPYSDEY

SEQ ID NO: 38 - protein, 323 aa, Homo sapiens
MDPKYQRVELNDGHFMPVLGFGTYAPPEVPRNRAVEVTKLAIEAGFRHIDSAYLYNNEEQVGLAIRSKIADGSVKREDIFYTSKLWCTFFQPQMVQPALESSLKKLQLDYVDLYLLHFPMALKPGETPLPKDENGKVIFDTVDLSATWEVMEKCKDAGLAKSIGVSNFNCRQLEMILNKPGLKYKPVCNQVECHPYLNQSKLLDFCKSKDIVLVAHSALGTQRHKLWVDPNSPVLLEDPVLCALAKKHKQTPALIALRYQLQRGVVVLAKSYNEQRIRENIQVFEFQLTSEDMKVLDGLNRNYRYVVMDFLMDHPDYPFSDEY

SEQ ID NO: 39 - protein, 493 aa, Homo sapiens
MISSKPRLVVPYGLKTLLEGISRAVLKTNPSNINQFAAAYFQELTMYRGNTTMDIKDLVKQFHQIKVEKWSEGTTPQKKLECLKEPGKTSVESKVPTQMEKSTDTDEDNVTRTEYSDKTTQFPSVYAVPGTEQTEAVGGLSSKPATPKTTTPPSSPPPTAVSPEFAYVPADPAQLAAQMLGKVSSIHSDQSDVLMVDVATSMPVVIKEVPSSEAAEDVMVAAPLVCSGKVLEVQVVNQTSVHVDLGSQPKENEAEPSTASSVPLQDEQEPPAYDQAPEVTLQADIEVMSTVHISSVYNDVPVTEGVVYIEQLPEQIVIPFTDQVACLKENEQSKENEQSPRVSPKSVVEKTTSGMSKKSVESVKLAQLEENAKYSSVYMEAEATALLSDTSLKGQPEVPAQLLDAEGAIKIGSEKSLHLEVEITSIVSDNTGQEESGENSVPQEMEGKPVLSGEAAEAVHSGTSVKSSSGPFPPAPEGLTAPEIEPEGESTAE

SEQ ID NO: 40 - protein, 524 aa, Homo sapiens
MPQLSLSWLGLGPVAASPWLLLLLVGGSWLLARVLAWTYTFYDNCRRLQCFPQPPKQNWFWGHQGLVTPTEEGMKTLTQLVTTYPQGFKLWLGPTFPLLILCHPDIIRPITSASAAVAPKDMIFYGFLKPWLGDGLLLSGGDKWSRHRRMLTPAFHFNILKPYMKIFNKSVNIMHDKWQRLASEGSARLDMFEHISLMTLDSLQKCVFSFESNCQEKPSEYIAAILELSAFVEKRNQQILLHTDFLYYLTPDGQRFRRACHLVHDFTDAVIQERRCTLPTQGIDDFLKNKAKSKTLDFIDVLLLSKDEDGKELSDEDIRAEADTFMFEGHDTTASGLSWVLYHLAKHPEYQEQCRQEVQELLKDREPIEIEWDDLAQLPFLTMCIKESLRLHPPVPVISRCCTQDFVLPDGRVIPKGIVCLINIIGIHYNPTVWPDPEVYDPFRFDQENIKERSPLAFIPFSAGPRNCIGQAFAMAEMKVVLALTLLHFRILPTHTEPRRKPELILRAEGGLWLRVEPLGANSQ

SEQ ID NO: 41 - protein, 423 aa, Homo sapiens
MRSLGANMAAALRAAGVLLRDPLASSSWRVCQPWRWKSGAAAAAVTTETAQHAQGAKPQVQPQKRKPKTGILMLNMGGPETLGDVHDFLLRLFLDRDLMTLPIQNKLAPFIAKRRTPKIQEQYRRIGGGSPIKIWTSKQGEGMVKLLDELSPNTAPHKYYIGFRYVHPLTEEAIEEMERDGLERAIAFTQYPQYSCSTTGSSLNAIYRYYNQVGRKPTMKWSTIDRWPTHHLLIQCFADHILKELDHFPLEKRSEVVILFSAHSLPMSVVNRGDPYPQEVSATVQKVMERLEYCNPYRLVWQSKVGPMPWLGPQTDESIKGLCERGRKNILLVPIAFTSDHIETLYELDIEYSQVLAKECGVENIRRAESLNGNPLFSKALADLVHSHIQSNELCSKQLTLSCPLCVNPVCRETKSFFTSQQL

SEQ ID NO: 42 - protein, 175 aa, Homo sapiens
MSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 43 - protein, 274 aa, Homo sapiens
MGTDSRAAKALLARARTLHLQTGNLLNWGRLRKKCPSTHSEELHDCIQKTLNEWSSQINPDLVREFPDVLECTVSHAVEKINPDEREEMKVSAKLFIVESNSSSSTRSAVDMACSVLGVAQLDSVIIASPPIEDGVNLSLEHLQPYWEELENLVQSKKIVAIGTSDLDKTQLEQLYQWAQVKPNSNQVNLASCCVMPPDLTAFAKQFDIQLLTHNDPKELLSEASFQEALQESIPDIQAHEWVPLWLLRYSVIVKSRGIIKSKGYILQAKRRGS

SEQ ID NO: 44 - protein, 522 aa, Homo sapiens
MALLPRALSAGAGPSWRRAARAFRGFLLLLPEPAALTRALSRAMACRQEPQPQGPPPAAGAVASYDYLVIGGGSGGLASARRAAELGARAAVVESHKLGGTCVNVGCVPKKVMWNTAVHSEFMHDHADYGFPSCEGKFNWRVIKEKRDAYVSRLNAIYQNNLTKSHIEIIRGHAAFTSDPKPTIEVSGKKYTAPHILIATGGMPSTPHESQIPGASLGITSDGFFQLEELPGRSVIVGAGYIAVEMAGILSALGSKTSLMIRHDKVLRSFDSMISTNCTEELENAGVEVLKFSQVKEVKKTLSGLEVSMVTAVPGRLPVMTMIPDVDCLLWAIGRVPNTKDLSLNKLGIQTDDKGHIIVDEFQNTNVKGIYAVGDVCGKALLTPVAIAAGRKLAHRLFEYKEDSKLDYNNIPTVVFSHPPIGTVGLTEDEAIHKYGIENVKTYSTSFTPMYHAVTKRKTKCVMKMVCANKEEKVVGIHMQGLGCDEMLQGFAVAVKMGATKADFDNTVAIHPTSSEELVTLR

SEQ ID NO: 45 - protein, 465 aa, Homo sapiens
MEPSSLELPADTVQRIAAELKCHPTDERVALHLDEEDKLRHFRECFYIPKIQDLPPVDLSLVNKDENAIYFLGNSLGLQPKMVKTYLEEELDKWAKIAAYGHEVGKRPWITGDESIVGLMKDIVGANEKEIALMNALTVNLHLLMLSFFKPTPKRYKILLEAKAFPSDHYAIESQLQLHGLNIEESMRMIKPREGEETLRIEDILEVIEKEGDSIAVILFSGVHFYTGQHFNIPAITKAGQAKGCYVGFDLAHAVGNVELYLHDWGVDFACWCSYKYLNAGAGGIAGAFIHEKHAHTIKPALVGWFGHELSTRFKMDNKLQLIPGVCGFRISNPPILLVCSLHASLEIFKQATMKALRKKSVLLTGYLEYLIKHNYGKDKAATKKPVVNIITPSHVEERGCQLTITFSVPNKDVFQELEKRGVVCDKRNPNGIRVAPVPLYNSFHDVYKFTNLLTSILDSAETKN

SEQ ID NO: 46 - protein, 572 aa, Homo sapiens
MEPEAPRRRHTHQRGYLLTRNPHLNKDLAFTLEERQQLNIHGLLPPSFNSQEIQVLRVVKNFEHLNSDFDRYLLLMDLQDRNEKLFYRVLTSDIEKFMPIVYTPTVGLACQQYSLVFRKPRGLFITIHDRGHIASVLNAWPEDVIKAIVVTDGERILGLGDLGCNGMGIPVGKLALYTACGGMNPQECLPVILDVGTENEELLKDPLYIGLRQRRVRGSEYDDFLDEFMEAVSSKYGMNCLIQFEDFANVNAFRLLNKYRNQYCTFNDDIQGTASVAVAGLLAALRITKNKLSDQTILFQGAGEAALGIAHLIVMALEKEGLPKEKAIKKIWLVDSKGLIVKGRASLTQEKEKFAHEHEEMKNLEAIVQEIKPTALIGVAAIGGAFSEQILKDMAAFNERPIIFALSNPTSKAECSAEQCYKITKGRAIFASGSPFDPVTLPNGQTLYPGQGNNSYVFPGVALGVVACGLRQITDNIFLTTAEVIAQQVSDKHLEEGRLYPPLNTIRDVSLKIAEKIVKDAYQEKTATVYPEPQNKEAFVRSQMYSTDYDQILPDCYSWPEEVQKIQTKVDQ

SEQ ID NO: 47 - protein, 605 aa, Homo sapiens
MMDLELPPPGLPSQQDMDLIDILWRQDIDLGVSREVFDFSQRRKEYELEKQKKLEKERQEQLQKEQEKAFFAQLQLDEETGEFLPIQPAQHIQSETSGSANYSQVAHIPKSDALYFDDCMQLLAQTFPFVDDNEVSSATFQSLVPDIPGHIESPVFIATNQAQSPETSVAQVAPVDLDGMQQDIEQVWEELLSIPELQCLNIENDKLVETTMVPSPEAKLTEVDNYHFYSSIPSMEKEVGNCSPHFLNAFEDSFSSILSTEDPNQLTVNSLNSDATVNTDFGDEFYSAFIAEPSISNSMPSPATLSHSLSELLNGPIDVSDLSLCKAFNQNHPESTAEFNDSDSGISLNTSPSVASPEHSVESSSYGDTLLGLSDSEVEELDSAPGSVKQNGPKTPVHSSGDMVQPLSPSQGQSTHVHDAQCENTPEKELPVSPGHRKTPFTKDKHSSRLEAHLTRDELRAKALHIPFPVEKIINLPVVDFNEMMSKEQFNEAQLALIRDIRRRGKNKVAAQNCRKRKLENIVELEQDLDHLKDEKEKLLKEKGENDKSLHLLKKQLSTLYLEVFSMLRDEDGKPYSPSEYSLQQTRDGNVFLVPKSKKPDVKKN

SEQ ID NO: 48 - protein, 274 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 49 - protein, 470 aa, Homo sapiens
MAGENHQWQGSILYNMLMSAKQTRAAPEAPETRLVDQCWGCSCGDEPGVGREGLLGGRNVALLYRCCFCGKDHPRQGSILYSMLTSAKQTYAAPKAPEATLGPCWGCSCGSDPGVGRAGLPGGRPVALLYRCCFCGEDHPRQGSILYSLLTSSKQTHVAPAAPEARPGGAWWDRSYFAQRPGGKEALPGGRATALLYRCCFCGEDHPQQGSTLYCVPTSTNQAQAAPEERPRAPWWDTSSGALRPVALKSPQVVCEAASAGLLKTLRFVKYLPCFQVLPLDQQLVLVRNCWASLLMLELAQDRLQFETVEVSEPSMLQKILTTRRRETGGNEPLPVPTLQHHLAPPAEARKVPSASQVQAIKCFLSKCWSLNISTKEYAYLKGTVLFNPDVPGLQCVKYIQGLQWGTQQILSEHTRMTHQGPHDRFIELNSTLFLLRFINANVIAELFFRPIIGTVSMDDMMLEMLCTKI

SEQ ID NO: 50 - protein, 560 aa, Homo sapiens
MGKWRPRGCCRGNMQCRQEVPATLTSSELFSTRNQPQPQPQPLLADAPVPWAVASRMCLTPGQGCGHQGQDEGPLPAPSPPPAMSSSRKDHLGASSSEPLPVIIVGNGPSGICLSYLLSGYTPYTKPDAIHPHPLLQRKLTEAPGVSILDQDLDYLSEGLEGRSQSPVALLFDALLRPDTDFGGNMKSVLTWKHRKEHAIPHVVLGRNLPGGAWHSIEGSMVILSQGQWMGLPDLEVKDWMQKKRRGLRNSRATAGDIAHYYRDYVVKKGLGHNFVSGAVVTAVEWGTPDPSSCGAQDSSPLFQVSGFLTRNQAQQPFSLWARNVVLATGTFDSPARLGIPGEALPFIHHELSALEAATRVGAVTPASDPVLIIGAGLSAADAVLYARHYNIPVIHAFRRAVDDPGLVFNQLPKMLYPEYHKVHQMMREQSILSPSPYEGYRSLPRHQLLCFKEDCQAVFQDLEGVEKVFGVSLVLVLIGSHPDLSFLPGAGADFAVDPDQPLSAKRNPIDVDPFTYQSTRQEGLYAMGPLAGDNFVRFVQGGALAVASSLLRKETRKPP

SEQ ID NO: 51 - protein, 483 aa, Homo sapiens
MAQADIALIGLAVMGQNLILNMNDHGFVVCAFNRTVSKVDDFLANEAKGTKVVGAQSLKEMVSKLKKPRRIILLVKAGQAVDDFIEKLVPLLDTGDIIIDGGNSEYRDTTRRCRDLKAKGILFVGSGVSGGEEGARYGPSLMPGGNKEAWPHIKTIFQGIAAKVGTGEPCCDWVGDEGAGHFVKMVHNGIEYGDMQLICEAYHLMKDVLGMAQDEMAQAFEDWNKTELDSFLIEITANILKFQDTDGKHLLPKIRDSAGQKGTGKWTAISALEYGVPVTLIGEAVFARCLSSLKDERIQASKKLKGPQKFQFDGDKKSFLEDIRKALYASKIISYAQGFMLLRQAATEFGWTLNYGGIALMWRGGCIIRSVFLGKIKDAFDRNPELQNLLLDDFFKSAVENCQDSWRRAVSTGVQAGIPMPCFTTALSFYDGYRHEMLPASLIQAQRDYFGAHTYELLAKPGQFIHTNWTGHGGTVSSSSYNA

SEQ ID NO: 52 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 53 - protein, 501 aa, Homo sapiens
MVRKPVVSTISKGGYLQGNVNGRLPSLGNKEPPGQEKVQLKRKVTLLRGVSIIIGTIIGAGIFISPKGVLQNTGSVGMSLTIWTVCGVLSLFGALSYAELGTTIKKSGGHYTYILEVFGPLPAFVRVWVELLIIRPAATAVISLAFGRYILEPFFIQCEIPELAIKLITAVGITVVMVLNSMSVSWSARIQIFLTFCKLTAILIIIVPGVMQLIKGQTQNFKDAFSGRDSSITRLPLAFYYGMYAYAGWFYLNFVTEEVENPEKTIPLAICISMAIVTIGYVLTNVAYFTTINAEELLLSNAVAVTFSERLLGNFSLAVPIFVALSCFGSMNGGVFAVSRLFYVASREGHLPEILSMIHVRKHTPLPAVIVLHPLTMIMLFSGDLDSLLNFLSFARWLFIGLAVAGLIYLRYKCPDMHRPFKVPLFIPALFSFTCLFMVALSLYSDPFSTGIGFVITLTGVPAYYLFIIWDKKPRWFRIMSEKITRTLQIILEVVPEEDKL

SEQ ID NO: 54 - protein, 137 aa, Homo sapiens
MGLRAGGTLGRAGAGRGAPEGPGPSGGAQGGSIHSGRIAAVHNVPLSVLIRPLPSVLDPAKVQSLVDTIREDPDSVPPIDVLWIKGAQGGDYFYSFGGCHRYAAYQQLQRETIPAKLVQSTLSDLRVYLGASTPDLQ

SEQ ID NO: 55 - protein, 337 aa, Homo sapiens
MSSSPVKRQRMESALDQLKQFTTVVADTGDFHAIDEYKPQDATTNPSLILAAAQMPAYQELVEEAIAYGRKLGGSQEDQIKNAIDKLFVLFGAEILKKIPGRVSTEVDARLSFDKDAMVARARRLIELYKEAGISKDRILIKLSSTWEGIQAGKELEEQHGIHCNMTLLFSFAQAVACAEAGVTLISPFVGRILDWHVANTDKKSYEPLEDPGVKSVTKIYNYYKKFSYKTIVMGASFRNTGEIKALAGCDFLTISPKLLGELLQDNAKLVPVLSAKAAQASDLEKIHLDEKSFRWLHNEDQMAVEKLSDGIRKFAADAVKLERMLTERMFNAENGK

SEQ ID NO: 56 - protein, 564 aa, Homo sapiens
MAELDLMAPGPLPRATAQPPAPLSPDSGSPSPDSGSASPVEEEDVGSSEKLGRETEEQDSDSAEQGDPAGEGKEVLCDFCLDDTRRVKAVKSCLTCMVNYCEEHLQPHQVNIKLQSHLLTEPVKDHNWRYCPAHHSPLSAFCCPDQQCICQDCCQEHSGHTIVSLDAARRDKEAELQCTQLDLERKLKLNENAISRLQANQKSVLVSVSEVKAVAEMQFGELLAAVRKAQANVMLFLEEKEQAALSQANGIKAHLEYRSAEMEKSKQELERMAAISNTVQFLEEYCKFKNTEDITFPSVYVGLKDKLSGIRKVITESTVHLIQLLENYKKKLQEFSKEEEYDIRTQVSAVVQRKYWTSKPEPSTREQFLQYAYDITFDPDTAHKYLRLQEENRKVTNTTPWEHPYPDLPSRFLHWRQVLSQQSLYLHRYYFEVEIFGAGTYVGLTCKGIDRKGEERNSCISGNNFSWSLQWNGKEFTAWYSDMETPLKAGPFRRLGVYIDFPGGILSFYGVEYDTMTLVHKFACKFSEPVYAAFWLSKKENAIRIVDLGEEPEKPAPSLVGTAP

SEQ ID NO: 57 - protein, 348 aa, Homo sapiens
MQFGELLAAVRKAQANVMLFLEEKEQAALSQANGIKAHLEYRSAEMEKSKQELETMAAISNTVQFLEEYCKFKNTEDITFPSVYIGLKDKLSGIRKVITESTVHLIQLLENYKKKLQEFSKEEEYDIRTQVSAIVQRKYWTSKPEPSTREQFLQYVHDITFDPDTAHKYLRLQEENRKVTNTTPWEHPYPDLPSRFLHWRQVLSQQSLYLHRYYFEVEIFGAGTYVGLTCKGIDQKGEERSSCISGNNFSWSLQWNGKEFTAWYSDMETPLKAGPFWRLGVYIDFPGGILSFYGVEYDSMTLVHKFACKFSEPVYAAFWLSKKENAIRIVDLGEEPEKPAPSLVGTAP

SEQ ID NO: 58 - protein, 105 aa, Homo sapiens
MVKQIESKTAFQEALDAAGDKLVVVDFSATWCGPCKMIKPFFHSLSEKYSNVIFLEVDVDDCQDVASECEVKCMPTFQFFKKGQKVGEFSGANKEKLEATINELV

SEQ ID NO: 59 - protein, 649 aa, Homo sapiens
  MOD_RES 648: note = pyrrolidone carboxylic acid
MGCAEGKAVAAAAPTELQTKGKNGDGRRRSAKDHHPGKTLPENPAGFTSTATADSRALLQAYIDGHSVVIFSRSTCTRCTEVKKLFKSLCVPYFVLELDQTEDGRALEGTLSELAAETDLPVVFVKQRKIGGHGPTLKAYQEGRLQKLLKMNGPEDLPKSYDYDLIIIGGGSGGLAAAKEAAQYGKKVMVLDFVTPTPLGTRWGLGGTCVNVGCIPKKLMHQAALLGQALQDSRNYGWKVEETVKHDWDRMIEAVQNHIGSLNWGYRVALREKKVVYENAYGQFIGPHRIKATNNKGKEKIYSAERFLIATGERPRYLGIPGDKEYCISSDDLFSLPYCPGKTLVVGASYVALECAGFLAGIGLDVTVMVRSILLRGFDQDMANKIGEHMEEHGIKFIRQFVPIKVEQIEAGTPGRLRVVAQSTNSEEIIEGEYNTVMLAIGRDACTRKIGLETVGVKINEKTGKIPVTDEEQTNVPYIYAIGDILEDKVELTPVAIQAGRLLAQRLYAGSTVKCDYENVPTTVFTPLEYGACGLSEEKAVEKFGEENIEVYHSYFWPLEWTIPSRDNNKCYAKIICNTKDNERVVGFHVLGPNAGEVTQGFAAALKCGLTKKQLDSTIGIHPVCAEVFTTLSVTKRSGASILQAGCXG

SEQ ID NO: 60 - protein, 494 aa, Homo sapiens
MFEIKKICCIGAGYVGGPTCSVIAHMCPEIRVTVVDVNESRINAWNSPTLPIYEPGLKEVVESCRGKNLFFSTNIDDAIKEADLVFISVNTPTKTYGMGKGRAADLKYIEACARRIVQNSNGYKIVTEKSTVPVRAAESIRRIFDANTKPNLNLQVLSNPEFLAEGTAIKDLKNPDRVLIGGDETPEGQRAVQALCAVYEHWVPREKILTTNTWSSELSKLAANAFLAQRISSINSISALCEATGADVEEVATAIGMDQRIGNKFLKASVGFGGSCFQKDVLNLVYLCEALNLPEVARYWQQVIDMNDYQRRRFASRIIDSLFNTVTDKKIAILGFAFKKDTGDTRESSSIYISKYLMDEGAHLHIYDPKVPREQIVVDLSHPGVSEDDQVSRLVTISKDPYEACDGAHAVVICTEWDMFKELDYERIHKKMLKPAFIFDGRRVLDGLHNELQTIGFQIETIGKKVSSKRIPYAPSGEIPKFSLQDPPNKKPKV

SEQ ID NO: 61 - DNA, 78 bp, Homo sapiens
  source 1..78: mol_type = genomic DNA
aaacctgccataactttcccaagaactgagtactctgtacctgggagtagttggcagatccactggtttctgactgga

SEQ ID NO: 62 - DNA, 78 bp, Homo sapiens
  source 1..78: mol_type = genomic DNA
tgtggacctaactagggggagcctaaaataatgttgggactacctagatggtcagaaagaatgagccaattaacttct

SEQ ID NO: 63 - DNA, 78 bp, Homo sapiens
  source 1..78: mol_type = genomic DNA
aaacctgccataactttcccaagaactgagtactctgtactacctagatggtcagaaagaatgagccaattaacttct

SEQ ID NO: 64 - DNA, 79 bp, Homo sapiens
  source 1..79: mol_type = genomic DNA
aggttaggtactgaactcatcaggaggctgaggttggaaagtagatttgacaaggttaagtaaaagaaaggcaaagctg

SEQ ID NO: 65 - DNA, 79 bp, Homo sapiens
  source 1..79: mol_type = genomic DNA
attttttcgggttttttttccacttttttccttttgaaattttattatttatttactcattttgagatagggtctcact

SEQ ID NO: 66 - DNA, 79 bp, Homo sapiens
  source 1..79: mol_type = genomic DNA
aggttgggtactgaactcatcaggaggctgagtttgaaattttattatttatttactcattttgagatagggtctcact

SEQ ID NO: 67 - DNA, 79 bp, Homo sapiens
  source 1..79: mol_type = genomic DNA
cttggttctcctgctactacttctgttgctgctacttgatccttacaggatgtttctatactttacaaaactctttggt

SEQ ID NO: 68 - DNA, 79 bp, Homo sapiens
  source 1..79: mol_type = genomic DNA
gtgatggcagtgggcacgcccatatacatttgcatacactctaatataaatgtttacaaacatacacacacacacattc

SEQ ID NO: 69 - DNA, 79 bp, Homo sapiens
  source 1..79: mol_type = genomic DNA
cttggttctcctgctactacttctgttgctgctacttgatctaatataaatgtttacaaacatacacacacacacattc

(19) United States
(12) Patent Application Publication
(10) Pub. No.: US 2025/0376733 A1
(43) Pub. Date: Dec. 11, 2025

(54) METHODS FOR DIAGNOSING AND TREATING CANCER BY MEANS OF THE EXPRESSION STATUS AND MUTATIONAL STATUS OF NRF2 AND DOWNSTREAM TARGET GENES OF SAID GENE

(71) Applicants: Genentech, Inc., South San Francisco, CA (US); Foundation Medicine, Inc., Cambridge, MA (US)

(72) Inventors: Christiaan Nicolaas KLIJN, San Francisco, CA (US); James LEE, San Bruno, CA (US); David Hugh STOKOE, San Francisco, CA (US); Robert GENTLEMAN, San Mateo, CA (US); Florian GNAD, Boston, MA (US); Leonard David GOLDSTEIN, Burlingame, CA (US); Juliann CHMIELECKI, Medford, MA (US); Ryan J. HARTMAIER, Cambridge, MA (US)

(21) Appl. No.: 19/016,860

(22) Filed: Jan. 10, 2025

Related U.S. Application Data
(63) Continuation of application No. 17/335,526, filed on Jun. 1, 2021, now abandoned.
Application No. 17/335,526 is a division of application No. 16/230,223, filed on Dec. 21, 2018, now Pat. No. 11,066,709.
Application No. 16/230,223 is a continuation of application No. PCT/US2017/041398, filed on Jul. 10, 2017.
(60) Provisional application No. 62/360,294, filed on Jul. 8, 2016.

(51) Int. Cl. C12Q 1/6886; A61K 31/337; A61K 45/06; A61P 35/00; C12Q 1/6806; C12Q 1/686; C12Q 1/6872

(52) U.S. Cl. CPC C12Q 1/6886; A61K 31/337; A61P 35/00; C12Q 1/6806; C12Q 1/686; C12Q 1/6872; A61K 45/06

(57) ABSTRACT

The invention provides methods of identifying a subject having cancer, such as lung cancer, by analyzing expression levels of one or more NRF2 splice variants or NRF2 target genes. The invention also provides methods of treating cancer in a subject with a NRF2 pathway antagonist, wherein the subject expresses one or more NRF2 splice variants or overexpresses one or more NRF2 target genes.

### SEQUENCE LISTING

The instant application contains a Sequence Listing which has been submitted electronically in XML format and is hereby incorporated by reference in its entirety. Said XML copy, created on Jan. 9, 2025, is named 50474-127004_Sequence_Listing_1_9_25 and is 152,294 bytes in size.

### FIELD OF THE INVENTION

The present invention relates generally to methods for diagnosing, treating, and providing prognoses for cancer, e.g., lung cancer.

### BACKGROUND OF THE INVENTION

Cancer remains one of the most deadly threats to human health. Lung cancer, in particular, is the primary cause of cancer-related death for men and women in the United States, despite recent advances in therapeutic treatments. The majority of lung cancers are non-small cell lung cancers (NSCLC), and most often of either the adenomatous or squamous subtype. Recent studies have identified patterns of point mutations that underlie these indications (Imielinski et al. Cell. 150 (6): 1107-1120, 2012), but despite an increasing number of identified mutations associated with various cellular pathways, a comprehensive understanding of the nature and influence of these mutations on these cellular pathways is lacking.

Thus, there is an unmet need in the field to develop effective diagnostic and therapeutic strategies for cancers, such as lung cancer.

### SUMMARY OF THE INVENTION

The present invention provides compositions and methods for diagnosing, treating, and providing prognoses for cancer, for example, lung cancer (e.g., non-small cell lung cancer (NSCLC)) and head and neck carcinoma.

In one aspect, the invention features a method of diagnosing a cancer in a subject, the method comprising: (a) determining the expression level of at least one (e.g., 1, 2, 3, 4, 5, 6, 7, 8, 9, 10, 11, 12, 13, 14, 15, 16, 17, 18, 19, 20, 21, 22, 23, 24, 25, 26, or 27) gene selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject; and (b) comparing the expression level of the at least one gene to a reference expression level of the at least one gene, wherein an increase in the expression level of the at least one gene in the sample relative to the reference expression level of the at least one gene identifies a subject having a cancer.

In another aspect, the invention features a method of identifying a subject having a cancer that is a NRF2-dependent cancer, the method comprising: (a) determining the expression level of at least one (e.g., 1, 2, 3, 4, 5, 6, 7, 8, 9, 10, 11, 12, 13, 14, 15, 16, 17, 18, 19, 20, 21, 22, 23, 24, 25, 26, or 27) gene selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject; (b) comparing the expression level of the at least one gene to a reference expression level of the at least one gene; and (c) determining if the subject's cancer is a NRF2-dependent cancer, wherein an increase in the expression level of the at least one gene in the sample relative to the reference expression level of the at least one gene identifies a subject having a NRF2-dependent cancer. In some embodiments of either of the preceding aspects, the expression level of at least two (e.g., 2, 3, 4, 5, 6, 7, 8, 9, 10, 11, 12, 13, 14, 15, 16, 17, 18, 19, 20, 21, 22, 23, 24, 25, 26, or 27) genes selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, and FTL in a sample obtained from the subject is determined. In some embodiments, the expression level of at least three (e.g., 3, 4, 5, 6, 7, 8, 9, 10, 11, 12, 13, 14, 15, 16, 17, 18, 19, 20, 21, 22, 23, 24, 25, 26, or 27) genes selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject is determined.

In some embodiments, the expression level of at least four (e.g., 4, 5, 6, 7, 8, 9, 10, 11, 12, 13, 14, 15, 16, 17, 18, 19, 20, 21, 22, 23, 24, 25, 26, or 27) genes selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject is determined. In some embodiments, the expression level of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject is determined.

In some embodiments, the expression level of one or more (e.g., 1, 2, 3, 4, 5, 6, 7, 8, 9, 10, 11, 12, 13, 14, 15, 16, 17, 18, 19, 20, or 21) of AKR1B10, AKR1C2, SRXN1, OSGIN1, FECH, GCLM, TRIM16,

KYNU, CABYR, SLC7A11, TRIM16L, AKR1C4, NROB1, UGDH, TXNRD1, GSR, AKR1C3, TALDO1, PGD, TXN, or NQO1 is determined. In some embodiments, the expression level of one or more (e.g., 1, 2, 3, 4, 5, 6, 7, 8, 9, 10, 11, or 12) of AKR1B10, AKR1C2, ME1, KYNU, CABYR, TRIM16L, AKR1C4, CYP4F11, RSPO3, AKR1B15, NROB1, and AKR1C3 is determined.

In some embodiments, (a) the expression level of the at least two genes in the sample is an average (e.g., mean or median) of the at least two genes of the sample; (b) the reference expression level of the at least two genes is an average (e.g., mean or median) of the at least two genes of the reference; and (c) the average (e.g., mean or median) of the at least two genes of the sample is compared to the average of the at least two genes of the reference.

In some embodiments, the reference expression level is the mean level of expression of the at least one gene in a population of subjects. In some embodiments, the population of subjects is a population of subjects sharing a common ethnicity.

In some embodiments, the reference expression level is the mean level of expression of the at least one gene in a population of subjects having cancer (e.g., lung cancer, e.g., non-small cell lung cancer (NSCLC), e.g., squamous NSCLC).

In some embodiments, the expression level is an mRNA expression level. In some embodiments, the mRNA expression level is determined by PCR, RT-PCR, RNA-seq, gene expression profiling, serial analysis of gene expression, or microarray analysis.

In other embodiments, the expression level is a protein expression level. In some embodiments, the protein expression level is determined by western blot, immunohistochemistry, or mass spectrometry. In some embodiments, any of the preceding methods further comprises determining a DNA sequence of NRF2. In some embodiments, the DNA sequence is determined by PCR, exome-seq, microarray analysis, or whole genome sequencing.

In another aspect, the invention features a method of diagnosing a cancer in a subject, the method comprising determining a DNA sequence of in a sample obtained from the subject, wherein the presence of NRF2 DNA comprising a deletion of all or a portion of its exon 2 identifies the subject as having a cancer. In some embodiments, the DNA sequence is determined by PCR, exome-seq, microarray analysis, or whole genome sequencing.

In another aspect, the invention features a method of identifying a subject having cancer, the method comprising determining the mRNA expression level of NRF2 comprising a deletion of all or a portion of its exon 2 in a sample obtained from the subject, wherein the presence of NRF2 comprising a deletion of all or a portion of its exon 2 identifies the subject as having a cancer. In some embodiments, the mRNA expression level is determined by PCR, RT-PCR, RNA-seq, gene expression profiling, serial analysis of gene expression, or microarray analysis. In some embodiments, the method further comprises determining a DNA sequence of the NRF2. In some embodiments, the DNA sequence is determined by PCR, exome-seq, microarray analysis, or whole genome sequencing.

In some embodiments of any of the preceding aspects, the NRF2 further comprises a deletion of all or a portion of its exon 3.

In another aspect, the invention features a method of diagnosing a cancer in a subject, the method comprising determining the protein expression level of NRF2 comprising a deletion of all or a portion of its Neh2 domain in a sample obtained from the subject, wherein the presence of NRF2 comprising a deletion of all or a portion of its Neh2 domain identifies the subject as having a cancer.

In another aspect, the invention features a method of identifying a subject having cancer, the method comprising determining the protein expression level of NRF2 comprising a deletion of all or a portion of its Neh2 domain in a sample obtained from the subject, wherein the presence of NRF2 comprising a deletion of all or a portion of its Neh2 domain identifies the subject as having a cancer.

In some embodiments of any of the preceding aspects, the NRF2 further comprises a deletion in all or a portion of its Neh4 domain. In some embodiments, the protein expression is determined by western blot, immunohistochemistry, or mass spectrometry.

In some embodiments, the method further comprises administering to the subject a therapeutically effective amount of a NRF2 pathway antagonist. In some embodiments, the method further comprises administering to the subject a therapeutically effective amount of an anti-cancer agent. In other embodiments, the method comprises administering an anti-cancer agent and a NRF2 pathway antagonist. In some embodiments, the anti-cancer agent and the NRF2 pathway antagonist are co-administered. In other embodiments, the anti-cancer agent and the NRF2 pathway antagonist are sequentially administered. In some embodiments, the anti-cancer agent is selected from the group consisting of an anti-angiogenic agent, a chemotherapeutic agent, a growth inhibitory agent, a cytotoxic agent, and an immunotherapy. In some embodiments, the anti-angiogenic agent is a VEGF antagonist. In some embodiments, the NRF2 pathway antagonist is selected from the group consisting of a CREB antagonist, a CREB Binding Protein (CBP) antagonist, a Maf antagonist, an activating transcription factor 4 (ATF4) antagonist, a protein kinase C (PKC) antagonist, a Jun antagonist, a glucocorticoid receptor antagonist, a UbcM2 antagonist, a HACE1 antagonist, a c-Myc agonist, a SUMO agonist, a KEAP1 agonist, a CUL3 agonist, or a retinoic acid receptor α (RARα) agonist.

In another aspect, the invention features a method of treating a subject having a cancer, the method comprising administering to the subject a therapeutically effective amount of a NRF2 pathway antagonist, wherein the expression level of at least one (e.g., 1, 2, 3, 4, 5, 6, 7, 8, 9, 10, 11, 12, 13, 14, 15, 16, 17, 18, 19, 20, 21, 22, 23, 24, 25, 26, or 27) of the following genes AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject has been determined to be increased relative to a reference expression level of the at least one gene. In other embodiments, the expression level of at least two (e.g., 2, 3, 4, 5, 6, 7, 8, 9, 10, 11, 12, 13, 14, 15, 16, 17, 18, 19, 20, 21, 22, 23, 24, 25, 26, or 27) genes selected from the group consisting of AKR1B10, AKR1C2, SRXN1, OSGIN1, FECH, GCLM, TRIM16, ME1, KYNU, CABYR, SLC7A11,TRIM16L, AKR1C4, CYP4F11, RSPO3, ABCC2, AKR1B15, NR0B1, UGDH, TXNRD1, GSR, AKR1C3, TALDO1, PGD, TXN, NQO1, and FTL in a sample obtained from the subject is determined. In other embodiments, the expression level of at least three (e.g., 3, 4, 5, 6, 7, 8, 9, 10, 11, 12, 13, 14, 15, 16, 17, 18, 19, 20, 21, 22, 23, 24, 25, 26, or 27) genes selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject is determined. In other embodiments, the expression level of at least four (e.g., 4, 5, 6, 7, 8, 9, 10, 11, 12, 13, 14, 15, 16, 17, 18, 19, 20, 21, 22, 23, 24, 25, 26, or 27) genes selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject is determined. In other embodiments, the expression level of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, and FTL in a sample obtained from the subject is determined.

In some embodiments, the expression level of one or more (e.g., 1, 2, 3, 4, 5, 6, 7, 8, 9, 10, 11, 12, 13, 14, 15, 16, 17, 18, 19, 20, or 21) of AKR1B10, AKR1C2, SRXN1, OSGIN1, FECH, GCLM, TRIM16, KYNU, CABYR, SLC7A11, TRIM16L, AKR1C4, NROB1, UGDH, TXNRD1, GSR, AKR1C3, TALDO1, PGD, TXN, or NQO1 is determined. In other embodiments, the expression level of one or more (e.g., 1, 2, 3, 4, 5, 6, 7, 8, 9, 10, 11, or 12) of AKR1B10, AKR1C2, ME1, KYNU, CABYR, TRIM16L, AKR1C4, CYP4F11, RSPO3, AKR1B15, NROB1, and AKR1C3 is determined.

In some embodiments, (a) the expression level of at least two genes in the sample is an average of the at least two genes of the sample; (b) the reference expression level of the at least two genes is an average of the at least two genes of the reference; and (c) the average of the at least two genes of the sample is compared to the average of the at least two genes of the reference. In some embodiments, the reference expression level is the mean level of expression of the at least one gene in a population of subjects. In some embodiments, the population of subjects is a population of subjects sharing a common ethnicity. In some embodiments, the reference expression level is the mean level of expression of the at least one gene in a population of subjects having cancer.

In some embodiments, the lung cancer is a non-small cell lung cancer (NSCLC), e.g., squamous NSCLC.

In some embodiments, the expression level is an mRNA expression level. In some embodiments, the mRNA expression level is determined by PCR, RT-PCR, RNA-seq, gene expression profiling, serial analysis of gene expression, or microarray analysis. In some embodiments, the mRNA expression level is determined by RNA-seq.

In some embodiments, the method further comprises determining a DNA sequence of the NRF2 (e.g., by PCR, exome-seq, microarray analysis, or whole genome sequencing).

In some embodiments, the expression level is a protein expression level. In some embodiments, the protein expression is determined by western blot, immunohistochemistry, or mass spectrometry.

In another aspect, the invention features a method of treating a subject having a cancer, the method comprising: (a) determining the mRNA expression level of NRF2 comprising a deletion of all or a portion of its exon 2 in a sample obtained from the subject, wherein the presence of NRF2 mRNA comprising a deletion of all or a portion of its exon 2 identifies the subject as having a cancer; and (b) administering to the subject a therapeutically effective amount of a NRF2 pathway antagonist.

In some embodiments, the mRNA expression is determined by PCR, RT-PCR, RNA-seq, gene expression profiling, serial analysis of gene expression, or microarray analysis. In some embodiments, the mRNA expression is determined by RNA-seq. In some embodiments, the method further comprises determining a DNA sequence of the NRF2 (e.g., by PCR, exome-seq, microarray analysis, or whole genome sequencing).

In another aspect, the invention features a method of treating a subject having a cancer, the method comprising: (a) determining a DNA sequence of NRF2 comprising a deletion of all or a portion of its exon 2 in a sample obtained from the subject, wherein the presence of NRF2 DNA comprising a deletion of all or a portion of its exon 2 identifies the subject as having a cancer; and (b) administering to the subject a therapeutically effective amount of a NRF2 pathway antagonist. In some embodiments, the DNA sequence is determined by PCR, exome-seq, microarray analysis, or whole genome sequencing. In some embodiments, the NRF2 (e.g., mRNA or DNA) further comprises a deletion in all or a portion of its exon 3.

In another aspect, the invention features a method of treating a subject having a cancer, the method comprising: (a) determining the protein expression level of NRF2 comprising a deletion of all or a portion of its Neh2 in a sample obtained from the subject, wherein the presence of NRF2 protein comprising a deletion of all or a portion of its Neh2 identifies the subject as having a cancer; and (b) administering to the subject a therapeutically effective amount of a NRF2 pathway antagonist.

In some embodiments, the NRF2 protein further comprises a deletion of all or a portion of its Neh4 domain. In some embodiments, the protein expression is determined by western blot, immunohistochemistry, or mass spectrometry. In some embodiments, the method further comprises determining a DNA sequence of the NRF2 (e.g., by PCR, exome-seq, microarray analysis, or whole genome sequencing).

In some embodiments, the method comprises administering to the subject a therapeutically effective amount of an anti-cancer agent. In some embodiments, the anti-cancer agent and the NRF2 pathway antagonist are co-administered. In other embodiments, the anti-cancer agent and the NRF2 pathway antagonist are sequentially administered. In some embodiments, the anti-cancer agent is selected from the group consisting of an anti-angiogenic agent, a chemotherapeutic agent, a growth inhibitory agent, a cytotoxic agent, and an immunotherapy. In some embodiments, the anti-angiogenic agent is a VEGF antagonist. In some embodiments, the NRF2 pathway antagonist is selected from the group consisting of a CREB antagonist, a CREB Binding Protein (CBP) antagonist, a Maf antagonist, an activating transcription factor 4 (ATF4) antagonist, a protein kinase C (PKC) antagonist, a Jun antagonist, a glucocorticoid receptor antagonist, a UbcM2 antagonist, a HACE1 antagonist, a c-Myc agonist, a SUMO agonist, a KEAP1 agonist, a CUL3 agonist, or a retinoic acid receptor α (RARα) agonist.

In some embodiments, the sample obtained from the subject is a tumor sample, e.g., from a biopsy sample. In some embodiments, the sample is obtained from a previously untreated subject. In some embodiments, the subject has a lung cancer (e.g., non-small cell lung cancer (NSCLC), e.g., squamous NSCLC) or a head and neck cancer (e.g., squamous head and neck cancer).

### BRIEF DESCRIPTION OF THE DRAWINGS

FIG. 1A is a plot showing 96 lung cancer cell lines subjected to RNA-seq, exome-seq, and SNP array analysis. Alterations in KRAS, TP53, KEAP1, EGFR, STK11, NFE2L2, and NF1 are shown.

FIG. 1B is a protein sequence representation showing point mutations in the NFE2L2 (NRF2) gene.

FIG. 1C is a protein sequence representation showing point mutations in the KEAP1 gene.

FIG. 1D is an image of the crystal structure of the KEAP1/NRF2 peptide complex.

FIG. 2A is a volcano plot illustrating the ratios of average expression levels for all genes in mutant (n=25) versus wild-type (WT) (n=74) KEAP1 NSCLC cell lines and the associated adjusted p-values resulting from the differential expression analysis. Significantly differentially expressed genes (>2-fold, p<0.01) are indicated, and gene sets previously identified as NRF2 targets are identified as black dots.

FIG. 2B is a heatmap showing the results of unsupervised ward clustering showing the upregulation of the 27 genes associated with KEAP1 mutations in NSCLC cell lines.

FIG. 3A is a heatmap showing the results of an unsupervised ward clustering showing that the NSCLC cell line-derived KEAP1 gene signature classified 32 of the 40 (80%) KEAP1 mutant lung adenocarcinomas from the cancer genome atlas (TCGA).

FIG. 3B is a heatmap showing the results of an unsupervised ward clustering showing that the NSCLC cell line-derived KEAP1 gene signature classifies 19 of 22 (86%) KEAP1 mutant and 27 of 27 (100%) NRF2 mutant lung squamous cell carcinomas from TCGA.

FIG. 4 is a graph showing the relative abundance of protein products of the KEAP1 gene signature in mutant (n=6) and WT (n=37) NSCLC cell lines.

FIG. 5 is a heatmap indicating the frequency of recurrent splice alterations seen in 19 tumor indications.

FIG. 6 shows the NRF2 exons and splice junctions predicted from RNA-seq data. Predicted features consistent with two annotated refGene transcripts are shown in gray. Identified exon-exon junctions corresponding to skip of exon 2 (J2, J5) or exon 2+3 (J3, J6) are shown in black and gray, respectively. A heatmap illustrates read evidence for exon-exon junctions (columns) across 482 TCGA lung squamous carcinoma (rows) on an FPKM scale after log2 (x +1) transformation.

FIG. 7 is a schematic depicting the effect of splice alterations in EGFR, NRF2, MET, and CTNNB1 on protein structure. Arrows indicate in-frame deletions as the result of the splice alteration.

FIG. 8A is a Venn diagram illustrating the mutual exclusive occurrence of NRF2 splice alteration and mutation in KEAP1 or NRF2 in squamous NSCLC.

FIG. 8B is a heatmap showing clustering of squamous NSCLC based on 27 candidate NRF2 target genes. Mutation status and NRF2 splice alteration are indicated for each sample.

FIG. 9A is a Venn diagram illustrating the mutual exclusive occurrence of NRF2 splice alteration and mutation in KEAP1 or NRF2 in head and neck cancers.

FIG. 9B is a heatmap showing clustering of head and neck cancers based on 27 candidate NRF2 target genes. Mutation status and NRF2 splice alteration are indicated for each sample.

FIG. 10 is a graph showing the presence of junction reads skipping exon 2 in KMS-27 and JHH-6 cells, as quantified by RNA-seq.

FIG. 11A is a schematic diagram showing the locations of exons within WT and exon 2-deleted NRF2 (Ae2 NRF2) mRNA, in relation to forward and reverse primers derived from exon 1 and exons 3/4, indicated by right-hand facing and left-hand facing arrows, respectively.

FIG. 11B is a series of agarose gel images showing RNA products amplified from total RNA of normal leucocytes, JHH-6 cells, and KMS-27 cells, by RT-PCR. Regions surrounding NRF2 exon 2 were amplified with the indicated primers. Fragments from wild-type NRF2, Ae2 NRF2, and primer dimers are indicated. Bands indicating the presence of Ae2 NRF2 RNA are visible in JHH-6 and KMS-27 cells.

FIG. 12A is a graph showing the sequencing results of the PCR products from JHH-6 and KMS-27 cells, indicating the deletion of exon 2 in NRF2.

FIG. 12B shows the nucleic acid and amino acid sequences for Ae2 NRF2.

FIG. 12C shows the nucleic acid and amino acid sequences for wild-type NRF2. The exon 2 sequence is shaded.

FIG. 13 shows the results of a Western blot experiment indicating the relative expression of phosphorylated NRF2, wild-type NRF2, and Δe2 NRF2 by HUH-1, JHH-6, and HuCCT1 cells. Protein lysates from the indicated cell lines were separated by SDS PAGE. * represents a likely non-specific band as it is not depleted by NRF2 siRNA transfection.

FIG. 14A shows the results of a Western blot experiment indicating the relative expression of phosphorylated NRF2, wild-type NRF2, and Ae2 NRF2 by HUH-1, JHH-6, and HuCCT1 cells in the presence and absence of lambda phosphatase (λ P′tase). Cells were grown in 6-well dishes and treated with 100 μg/ml cyclohexamide (CHX) for the indicated times. The lysates were either incubated with buffer or 400 units lambda phosphatase for 30 min, before separation by SDS PAGE and Western blotting with NRF2 antibodies.

FIG. 14B is a graph showing the stability of NRF2 protein expressed by HuCCT1 cells (circles), JHH-6 cells (squares), and HUH1 cells (triangles) in the presence of CHX. Band intensities from the results shown in FIG. 14A were quantified and fitted to a one-phase decay curve to obtain protein half-life estimates, which are indicated next to each curve. Relative protein expression was taken as a percent of initial concentration of each cell line.

FIG. 14C shows the results of a Western blot experiment indicating the relative expression of NRF2 and Ae2 NRF2 by HUH-1, JHH-6, and HuCCT1 cells after tranfection with either siNTC (50 nM) or siKEAP1 (50 nM). Cells were grown in 6-well dishes and treated with 100 μg/ml cyclohexamide (CHX) for the indicated times. The lysates were incubated with 400 units lambda phosphatase for 30 minutes, before separation by SDS PAGE and Western blotting with NRF2 antibodies.

FIG. 14D is a graph showing the stability of NRF2 protein expressed by HuCCT1 cells (circles), JHH-6 cells (squares), and HUH1 cells (triangles) in the presence of CHX after tranfection with siNTC (solid lines) or siKEAP1 (dashed lines). Band intensities from the results shown in FIG. 14C were quantified and fitted to a one-phase decay curve to obtain protein half-life estimates, which are indicated next to each curve. Relative protein expression was taken as a percent of initial concentration of each cell line.

FIG. 15 shows the results of a Western blot experiment indicating the expression of Δe2 NRF2 by KMS-27 cells. 20 μg lysates from HCC-1354, KMS-27, and HuCCT1 cells were prepared, and for all except HuCCT1 treated with λ P′tase. Untreated and treated lysates were then subjected to SDS PAGE, and NRF2 and actin were detected.

FIG. 16 shows the results of a Western blot experiment indicating nuclear localization of NRF2. HuCCT1, HUH-1, and JHH-6 cells were grown in 10 cm dishes and partitioned into nuclear and cytosol fractions. Fractions were separated by SDS PAGE and NRF2 was visualized. Nuclear and cytosolic purity was estimated using Hsp90 as a cytosolic marker and HDAC2 as a nuclear marker.

FIG. 17A is a graph showing the expression of the 27 signature NRF2 target genes of the KEAP1 gene signature (each displayed on the x-axis) in 16 hepatocellular carcinoma cell lines (represented by black squares, filled gray circles, and open gray circles) using RNA-seq data described in Klijn et al. (Nat Biotechnol. 33 (3): 306-312, 2014). Filled gray circles represent mutant KEAP1 liver cancer cell lines, and open gray circles represent the JHH-6 cell line.

FIG. 17B is a graph showing the expression of the 27 signature NRF2 target genes of the KEAP1 gene signature (each displayed on the x-axis) in 18 multiple myeloma cell lines (represented by black squares and open gray circles) using RNA-seq data described in Klijn et al. (Nat. Biotechnol. 33 (3): 306-312, 2014). Open gray circles represent the KMS-27 cell line.

FIG. 18A is a bar graph showing the NRF2 target gene score (mean z-scores for the 27 NRF2 target genes determined over the full data set) in the 16 hepatocellular carcinoma cell lines. KEAP1 and NRF2 alterations are indicated as filled and outlined boxes, respectively.

FIG. 18B is a bar graph showing the NRF2 target gene score (mean z-scores for the 27 NRF2 target genes determined over the full data set) in the 18 multiple myeloma cell lines. The outlined box indicates a NRF2 alteration.

FIG. 19 is a bar graph showing the viability of HUH-1, JHH-6, and HuCCT1 cells in the presence or absence of siRNAs targeting NRF2. Cells were seeded into 96-well plates containing either a non-targeted siRNA control (NTC), or siRNA targeting NRF2 (NRF2). Viability was measured 4 days later using CellTiter-Glo. Viability is presented as a percentage of NTC luminescence.

FIG. 20 is a series of bar graphs showing the effect of transfection reagents on relative NRF2 expression by HUH-1, JHH-6, and HuCCT1 cells. Cells were grown in 6-well dishes and transfected with siRNA targeting NRF2 exon 5 of NRF2. Total RNA was isolated after 48 hours, and NRF2 expression was measured using Taqman probes targeting exon 5.

FIG. 21 is a series of bar graphs showing the effect of transfection reagents on four well-characterized NRF2 target genes, SLC7A11, GCLC, NROB1, and SGRN, expressed by HUH-1 cells (dark gray shaded bars), JHH-6 cells (light gray shaded bars), and HuCCT1 cells (black shaded bars). Cells were grown in 6-well dishes and transfected with siRNA targeting NRF2exon 5 of NRF2, or non-targeted siRNA (NTC). Total RNA was isolated after 48 hours, and gene expression was measured using Taqman probes targeting the indicated NRF2 target genes.

FIG. 22 is a series of representative FACS histograms showing the effect of NRF2 targeting siRNA on DNA fragmentation in HUH-1, JHH-6, and HuCCT1 cells. Cells were treated with staurosporin as a positive control.

FIG. 23 is a set of immunoblots showing the effect of NRF2 exon 2 and exon 2+3 deletions on KEAP1 interaction. 293 cells were transfected with plasmids expressing FLAG-NRF2, Δe2 FLAG-NRF2, Δe2+3 FLAG-NRF2 or HA-KEAP1. 48 hours after transfection, cells were lysed, and either lysates (top gel) or anti-FLAG immunoprecipitations were analyzed by Western blotting using the indicated antibodies.

FIG. 24A is a set of immunoblots showing the effect of cyclohexamide on NRF2 stability. 293 cells were transfected with the same plasmids as described in FIG. 23 and treated with 100 μg/ml cycloheximide (CHX) for the indicated times. Cells were lysed and separated by SDS PAGE, and Western blotted using NRF2 and anti-actin antibodies.

FIG. 24B is a graph showing the stability of truncated NRF2 following KEAP1 expression over time.

FIG. 25 is a series of bar graphs showing the expression of various NRF2 target genes under various transfection conditions. Cells were treated as in FIGS. 24A-24B but harvested for total RNA, which was used to analyze the expression of the indicated genes using Taqman RT-PCR.

FIGS. 26A-1 to 26B-2 are a series of graphs showing the mRNA expression levels of indicated NRF2 target genes in TCGA squamous NSCLC tumors, plotted according to mutation status of KEAP1 and NRF2. Individual graphs show mRNA expression levels of NQO1 (FIG. 26A-1), SLC7A11 (FIG. 26B-1), KYNU (FIG. 26C-1), FECH (FIG. 26D-1), CABYR (FIG. 26E-1), GCLM (FIG. 26F-1), TXN (FIG. 26G-1), AKR1C4 (FIG. 26H-1), AKR1C3 (FIG. 261-1), TXNRD1 (FIG. 26J-1), SRXN1 (FIG. 26K-1), GPX2 (FIG. 26L-1), AKR1C2 (FIG. 26M-1), OSGIN1 (FIG. 26N-1), TRIM16 (FIG. 26O-1), NROB1 (FIG. 26P-1), GSR (FIG. 26Q-1), AKR1B10 (FIG. 26R-1), TRIM16L (FIG. 26S-1), PGD (FIG. 26T-1), ME1 (FIG. 26U-1), FTL (FIG. 26V-1), RSPO3 (FIG. 26W-1), CYP4F11 (FIG. 26X-1), UGDH (FIG. 26Y-1), TALDO1 (FIG. 26Z-1), ABCC2 (FIG. 26A-2), and AKR1B15 (FIG. 26B-2). Only samples for which both exome-seq and RNA-seq data were available were considered. One sample with mutations in both NRF2 and KEAP1 was excluded. In addition, samples with evidence for NRF2 copy number changes |log2 (CAN)|>0.5 were excluded.

FIG. 27A is an exome-seq graph showing relative NRF2 exon abundance across 808 cancer cell lines, showing a decrease in reads mapping to exon 2.

FIG. 27B is an exome-seq graph showing normalized z-scores for exon read coverage across 1,218 squamous NSCLC tumors. Eleven tumors showing decreased read count for exon 2 or exon 2+3 are compared to nearby control regions.

FIG. 28A is a schematic diagram showing the genomic location of discordant read pairs in seven tumors supporting genomic alterations affecting NRF2 exon 2 or exon 2+3.

FIG. 28B-1 is a set of graphs showing the copy number analyses of chromosome 2 showing two tumor samples with NRF2 exon 2 focal deletions. Arrows point to NRF2 exon 2. The log-ratio of target regions are shown in black and control regions are shown in gray.

FIG. 28B-2 is a set of graphs showing the copy number analyses of chromosome 2 showing two tumor samples with NRF2 exon 2+3 focal deletions. Arrows point to NRF2 exon 2 and exon 3. The log-ratio of target regions are shown in black and control regions are shown in gray.

FIG. 28C is a series of whole-genome sequencing graphs showing the presence of microdeletions surrounding NRF2 exon 2 in JHH-6 cells, KMS-27 cells, as well as primary tumor and adjacent matched DNA. The sequences of reads spanning the deletions are shown NRF2NRF2.

FIG. 29 is a series of agarose gel images showing RNA products amplified from total RNA of select patients with squamous NSCLC. Shown are amplification products from patient #58 tumor tissue, patient #64 tumor tissue, patient #63 normal tissue, and patient #63 tumor tissue by RT-PCR. Regions surrounding NRF2 exon 2 were amplified with the primers indicated in FIG. 11A. Fragments from wild-type NRF2 and Δe2 NRF2 are indicated. RT-PCR analysis identified patient #63 as having loss of NRF2 exon 2, which was strongly enriched in the tumor compared to the adjacent normal tissue.

FIG. 30 is a graph showing the presence of junction reads skipping exon 2 in tumor and normal cells, as quantified by RNA-seq.

FIG. 31 is a histogram of the mutant KEAP1 gene signature score for TCGA samples from lung squamous carcinoma (LUSC). Dark gray histograms represent KEAP1/NRF2 mutant tumors, light gray histograms represent exon 2/3-deleted tumors, and medium gray histograms represent KEAP1/NRF2 wild-type tumors. The gene signature score for a given sample was determined by summation of gene expression z-scores over all genes in the gene signature.

FIG. 32 is a series of histograms of the mutant KEAP1 gene signature score for TCGA samples from lung squamous carcinoma (LUSC), lung adenoma (LUAD), and head and neck squamous carcinoma (HNSC). Dark gray histograms represent KEAP1/NRF2 mutant tumors, light gray histograms represent exon 2/3-deleted tumors, and medium gray histograms represent KEAP1/NRF2 wild-type tumors. The gene signature score for a given sample was determined by summation of gene expression z-scores over all genes in the gene signature.

FIG. 33 is a series of histograms of the mutant KEAP1 gene signature score for TCGA samples from lung squamous carcinoma (LUSC), lung adenoma (LUAD), and head and neck squamous carcinoma (HNSC). Dark gray histograms represent tumor samples, and light gray histograms represent normal samples. The gene signature score for a given sample was determined by summation of gene expression z-scores over all genes in the gene signature.

FIG. 34 is a series of junction read sequences showing the structure of the deletions in JHH-6 cells, KMS-26 cells, and primary tumor, identified by WGS. The DNA sequences of the 3′ end, 5′ end, and junction read of JHH-6 cells are provided by SEQ ID NOs: 61-63, respectively. The DNA sequences of the 3′ end, 5′ end, and junction read of KMS-27 cells are provided by SEQ ID NOs: 64-66, respectively. The DNA sequences of the 3′ end, 5′ end, and junction read of primary tumor cells are provided by SEQ ID NOs: 67-69, respectively.

FIG. 35 is a series of Western blots showing the relative expression of NRF2. The indicated cell lines were infected with lentiviruses expressing independent non-target control (NTC) or three independent NRF2 shRNA sequences (sh1, sh2, and sh3) and were incubated for 48 hours with (+) or without (−) 500 ng/ml doxycycline (dox) following puromycin selection.

FIG. 36 is a graph showing the viability of the cell lines shown in FIG. 35 after incubation with or without dox for 7 days. Viability was measured using CellTiter-Glo (CTG) ATP detection. Each circle is the average of six technical replicates, and values were normalized to the average percent viability of three independent NTCs +dox.

FIG. 37 is a graph showing the viability of cell lines treated with dox vs no dox. Cells were grown for four days and viability measured using CTG ATP measurement. Significance was calculated using Student's t test.

FIG. 38 is a graph showing the viability of 28 NSCLC cell lines following treatment with NRF2 siRNA relative to NTC treatment. Cells are grouped by KEAP1 genotype. Significance was calculated using Student's t test.

FIG. 39 is a Western blot experiment showing the expression of NRF2 in KEAP1 mutant tumors. Mice were implanted with A549 cells expressing NRF2 sh10. When tumors reached ˜200 mm3, 1 mg/ml doxycycline or 5% sucrose was added to the drinking water. After five days, tumor extracts were blotted for NRF2.

FIG. 40 is a Western blot experiment showing the expression of NRF2 in KEAP1 wild-type tumors. Mice were implanted with H441 cells expressing NRF2 sh10. When tumors reached ˜200 mm3, 1 mg/ml doxycycline or 5% sucrose was added to the drinking water. After five days, tumor extracts were blotted for NRF2.

FIG. 41A is a graph showing the kinetics of tumor volume in mice implanted with KEAP1 mutant tumors. Mice were implanted with A549 cell lines expressing NRF2 sh10. When tumors reached ˜200 mm³, mice were randomized into groups of 10, and either 1 mg/ml doxycycline or 5% sucrose was added to the drinking water. Tumors were measured over a 28-day period. Error bars represent SEM (n=10).

FIG. 41B is a graph showing the kinetics of tumor volume in mice implanted with KEAP1 wild-type tumors. Mice were implanted with H441 cell lines expressing NRF2 sh10. When tumors reached ˜200 mm³, mice were randomized into groups of 10, and either 1 mg/ml doxycycline or 5% sucrose was added to the drinking water. Tumors were measured over a 28-day period. Error bars represent SEM (n=10).

FIG. 42 is a series of bar graphs showing viability of A549 or H441 cells in various growth conditions. A549 and H460 cells expressing NTC or NRF2 sh10 shRNAs were plated into either 2D tissue culture treated plastic dishes or ultra-low attachment (ULA) coated tissue culture plates. They were then cultured for five days in either environmental oxygen concentrations or 0.5% oxygen (hypoxia). Cell viability was assessed by CTG ATP measurements.

FIG. 43 is a series of photographs showing colony formation of KEAP1 mutant cell lines (A549, H1437, and H460) and KEAP1 wild-type cell lines (H1048, H441, and Calu6) in soft agar treated with vehicle, 500 ng/ml dox, or 1 mM reduced glutathione (GSH). Representative areas of the plate were photographed. FIG. 44 is a series of bar graphs showing the quantified colony formation for each cell type and treatment group shown in FIG. 43. Error bars represent standard deviation from biological triplicate wells.

FIG. 45 is a series of photographs showing A549 colony formation on SCIVAX® micropatterned nanoculture dishes. Cells were photographed after about five days in culture in the presence or absence of 500 ng/ml dox.

FIG. 46 is a bar graph showing viability of the cells from FIG. 45, quantified by CTG ATP measurements. The left column of each treatment group represents 1,000-cell cultures, and the right column represents 5,000-cell cultures.

FIG. 47 is a series of photographs showing 5,000, 50,000, or 500,000 NTC or NRF2sh10 shRNA expressing A549 cells plated in methylcellulose-containing tissue culture dishes. Cells were photographed after ˜10 days of culture in the presence or absence of 500 ng/ml doxycycline.

FIG. 48 is a series of photographs showing A549 cells expressing NRF2sh10 shRNA plated into regular tissue culture dishes (top) or soft agar (bottom). Cells were treated with either vehicle or 500 ng/ml doxycycline, in the presence or absence of 2 mM N-acetyl cysteine (NAC). Viability in 2D growth was measured after about five days by CTG ATP measurement, and photographs of cells in soft agar were taken after about ten days of growth. FIG. 49 is bar graph showing reactive oxygen species (ROS) levels under indicated conditions as measured using 2′,7′-dichlorodihydrofluorescein diacetate (H2DCF). Error bars represent standard deviation from triplicate wells. FIG. 50 is a Western blot experiment showing the effect on NRF2 knockdown on expression of

SLC7A11. A549 cells expressing NRF2 sh10 were treated with vehicle or 500 ng/ml dox for the indicated time points and blotted using SLC7A11 and β-actin antibodies.

FIG. 51 is a bar graph showing cystine uptake by A549 cells expressing NTC1 or NRF2 sh10 over various concentrations of erastin. A549 cells expressing NTC1 or NRF2 sh10 were incubated with vehicle or dox for 48 hours, then incubated with 0.5 uCi ¹⁴C-Cystine for 20 minutes. Cells were lysed and intracellular cystine was measured by liquid scintillation counting.

FIG. 52 is a bar graph showing glutathione (GSH) levels in A549 and H1437 cells in response to NRF2 knockdown.

FIG. 53 is a histogram showing increasing ROS levels in response to shNRF2 and/or erastin, as measured by H2DCF.

FIG. 54 is a graph showing viability of A549 cells expressing shNTC or shNFR2 over a dose response of erastin after about four days, as measured using CTG ATP measurements.

FIG. 55A is a graph showing the IC₅₀ of erastin on KEAP1 wild-type cell lines versus KEAP1 mutant cell lines, derived from a dose response graph as shown in FIG. 54.

FIG. 55B is a graph showing the viability of KEAP1 wild-type cell lines versus KEAP1 mutant cell lines in response to erastin, as area under the curve of a dose response graph as shown in FIG. 54.

FIG. 56A is a graph showing the IC₅₀ of the glutaminase inhibitor BPTES on KEAP1 wild-type cell lines versus KEAP1 mutant cell lines.

FIG. 56B is a graph showing the viability of KEAP1 wild-type cell lines versus KEAP1 mutant cell lines in response to the glutaminase inhibitor BPTES.

FIG. 57A is a graph showing the IC₅₀ of the glutathione synthase inhibitor buthionine sylphoximine (BSO) on KEAP1 wild-type cell lines versus KEAP1 mutant cell lines.

FIG. 57B is a graph showing the viability of KEAP1 wild-type cell lines versus KEAP1 mutant cell lines in response to BSO.

FIG. 58 is a scatterplot showing average gRNA expression per indicated gene in KEAP1 mutant

NSCLC cells grown for 15 days in a 3D methylcellulose culture versus a 2D plastic tissue culture dish. A549 cells were infected with lentivirus (0.3 MOI at 1000× coverage) expressing a gRNA library comprising 481 NRF2/KEAP1 target genes and 37 control genes. Puromycin-resistant cells were then plated into 2D plastic tissue culture dishes or grown in methyl cellulose. After various time points, cells were collected and gRNAs identified by Next Gen sequencing.

FIG. 59 is a scatterplot showing average gRNA expression per indicated gene in KEAP1 mutant NSCLC cells implanted in nude mice (xeno) versus grown for 15 days in a 2D plastic tissue culture dish. A549 cells were infected with lentivirus (0.3 MOI at 1000× coverage) expressing a gRNA library comprising 481 NRF2/KEAP1 target genes and 37 control genes. Puromycin-resistant cells were then plated into 2D plastic tissue culture dishes or implanted into nude mice. After various time points, cells were collected and gRNAs identified by Next Gen sequencing.

FIG. 60 is a scatterplot showing average gRNA expression per indicated gene in KEAP1 mutant NSCLC cells implanted in nude mice (xeno) versus grown for 15 days in a 3D methylcellulose culture. A549 cells were infected with lentivirus (0.3 MOI at 1000× coverage) expressing a gRNA library comprising 481 NRF2/KEAP1 target genes and 37 control genes. Puromycin-resistant cells were then grown in methyl cellulose or implanted into nude mice. After various time points, cells were collected and gRNAs identified by Next Gen sequencing.

FIG. 61 is a graph showing kinetics of A549 xenograft tumor volume in response to treatment with the Erb2 antibody YW57.88.5.

FIG. 62 is a series of photographs showing colony formation of KEAP1 mutant cell lines and KEAP1 wild-type cell lines grown in soft agar (anchorage independent conditions) in the presence of IGF1R inhibitors linsitinib and NVP-AEW541, and in the presence or absence of glutathione.

### DETAILED DESCRIPTION OF EMBODIMENTS OF THE INVENTION

### I. Introduction

The present invention provides diagnostic and accompanying therapeutic methods for cancer, such as lung cancer (e.g., NSCLC) or head and neck squamous cancer (e.g., HNSC). The invention is based, at least in part, on the discovery that splice variants in NRF2 that remove exon 2 or exons 2+3 result in an unexpected mechanism for conferring NRF2 activation in cancers. The NRF2 splice variants result in NRF2 activation by a mutually exclusive mechanism from mutations in KEAP1 or NRF2, yet result in a similar NRF2 target gene expression profile. In cell lines with microdeletions that result in these NRF2 splice variants, there is a loss of NRF2-KEAP1 interaction, increased NRF2 stabilization, induction of a NRF2 transcriptional response, and NRF2 pathway dependency. This occurs in 3-6% of squamous NSCLC and 1-2% of HNSC and results in a similar activation of NRF2 target genes and dependency on the pathway as KEAP1 mutations.

This discovery is useful for diagnosing a subject suffering from cancer (e.g., by detecting a NRF2 splice variant or by detecting a gene or protein expression profile consistent with the presence of a NRF2 splice variant) and for treating a subject according to such a diagnosis (e.g., by administering a therapeutically effective amount of a NRF2 pathway antagonist, e.g., a cAMP Responsive Element Binding Protein (CREB) Binding Protein (CBP) inhibitor).

### II. Definitions

The terms “diagnose,” “diagnosing,” or “diagnosis” are used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, e.g., by histopathological criteria, or by molecular features (e.g., a subtype characterized by expression of one or a combination of biomarkers (e.g., particular genes or proteins encoded by said genes)).

The terms “cancer” and “cancerous” refer to or describe the physiological condition in mammals that is typically characterized by unregulated cell growth. Included in this definition are benign and malignant cancers as well as dormant tumors or micrometastatses. Examples of cancer include, but are not limited to, carcinoma, lymphoma, blastoma, glioblastoma, sarcoma, and leukemia. Cancers may include, for example, breast cancer, squamous cell cancer, lung cancer (including small-cell lung cancer, non-small cell lung cancer (NSCLC), adenocarcinoma of the lung, and squamous carcinoma of the lung (e.g., squamous NSCLC)), various types of head and neck cancer (e.g., HNSC), cancer of the peritoneum, hepatocellular cancer, gastric or stomach cancer (including gastrointestinal cancer), pancreatic cancer, ovarian cancer, cervical cancer, liver cancer, bladder cancer, hepatoma, colon cancer, colorectal cancer, endometrial or uterine carcinoma, salivary gland carcinoma, kidney or renal cancer, liver cancer, prostate cancer, vulval cancer, thyroid cancer, and hepatic carcinoma, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

A “patient” or “subject” herein refers to any single animal (including, e.g., a mammal, such as a dog, a cat, a horse, a rabbit, a zoo animal, a cow, a pig, a sheep, a non-human primate, and a human), such as a human, eligible for treatment who is experiencing or has experienced one or more signs, symptoms, or other indicators of a disease or disorder, such as a cancer. Intended to be included as a patient are any patients involved in clinical research trials not showing any clinical sign of disease, patients involved in epidemiological studies, or patients once used as controls. The patient may have been previously treated with a NRF2 pathway antagonist or another drug, or not so treated. The patient may be naive to an additional drug(s) being used when the treatment herein is started, i.e., the patient may not have been previously treated with, for example, a therapy other than a NRF2 pathway antagonist (e.g., a VEGF antagonist or a PD-1 axis binding antagonist) at “baseline” (i.e., at a set point in time before the administration of a first dose of a NRF2 pathway antagonist in the treatment method herein, such as the day of screening the subject before treatment is commenced). Such “naive” patients or subjects are generally considered to be candidates for treatment with such additional drug(s).

The terms “level of expression” or “expression level” in general are used interchangeably and generally refer to the amount of a biomarker in a biological sample. “Expression” generally refers to the process by which information (e.g., gene-encoded and/or epigenetic information) is converted into the structures present and operating in the cell. Therefore, as used herein, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, transfer and ribosomal RNAs).

The terms “biomarker” and “marker” are used interchangeably herein to refer to a DNA, RNA, protein, carbohydrate, or glycolipid-based molecular marker, the expression or presence of which in a subject's or patient's sample can be detected by standard methods (or methods disclosed herein). Such biomarkers include, but are not limited to, the mRNA sequences set forth in Table 1 and encoded proteins thereof. Expression of such a biomarker may be determined to be higher or lower in a sample obtained from a patient sensitive or responsive to a NRF2 pathway antagonist than a reference level (including, e.g., the average (e.g., mean or median) expression level of the biomarker in a sample from a group/population of patients, e.g., patients having cancer, and being tested for responsiveness to a NRF2 pathway antagonist; the median expression level of the biomarker in a sample from a group/population of patients, e.g., patients having cancer, and identified as not responding to NRF2 pathway antagonists; the level in a sample previously obtained from the individual at a prior time; or the level in a sample from a patient who received prior treatment with a NRF2 pathway antagonist in a primary tumor setting, and who now may be experiencing metastasis). Individuals having an expression level that is greater than or less than the reference expression level of at least one gene, such as those set forth in Table 1 can be identified as subjects/patients likely to respond to treatment with a NRF2 pathway antagonist. For example, such subjects/patients who exhibit gene expression levels at the most extreme 50%, 45%, 40%, 35%, 30%, 25%, 20%, 15%, 10%, or 5% relative to (i.e., higher or lower than) the reference level (such as the mean level), can be identified as subjects/patients (e.g., patients having cancer) likely to respond to treatment with a NRF2 pathway antagonist.

TABLE 1

| SEQ ID NO | Biomarker |
|---|---|
| 1 | ABCC2 |
| 2 | AKR1B10 |
| 3 | AKR1B15 |
| 4 | AKR1C2 |
| 5 | AKR1C3 |
| 6 | AKR1C4 |
| 7 | CABYR |
| 8 | CYP4F11 |
| 9 | FECH |
| 10 | FTL |
| 11 | GCLM |
| 12 | GSR |
| 13 | KYNU |
| 14 | ME1 |
| 15 | NRF2/NFE2L2 |
| 16 | NQO1 |
| 17 | NR0B1 |
| 18 | OSGIN1 |
| 19 | PGD |
| 20 | RSPO3 |
| 21 | SLC7A11 |
| 22 | SRXN1 |
| 23 | TALDO1 |
| 24 | TRIM16 |
| 25 | TRIM16L |
| 26 | TXN |
| 27 | TXNRD1 |
| 28 | UGDH |

The term “ABCC2” as used herein, refers to any native ABCC2 (ATP-Binding Cassette Sub-Family C, Member 2) from any vertebrate source, including mammals such as primates (e.g., humans) and rodents (e.g., mice and rats), unless otherwise indicated. The term encompasses “full-length,” unprocessed ABCC2 as well as any form of ABCC2 that results from processing in the cell. The term also encompasses naturally occurring variants of ABCC2, e.g., splice variants or allelic variants. The nucleic acid sequence of an exemplary human ABCC2 is set forth in SEQ ID NO: 1. The amino acid sequence of an exemplary protein encoded by human ABCC2 is shown in SEQ ID NO: 33.

The term “AKR1B10” as used herein, refers to any native AKR1B10 (Aldo-Keto Reductase Family 1, Member B10) from any vertebrate source, including mammals such as primates (e.g., humans) and rodents (e.g., mice and rats), unless otherwise indicated. The term encompasses “full-length,” unprocessed AKR1B10 as well as any form of AKR1B10 that results from processing in the cell. The term also encompasses naturally occurring variants of AKR1B10, e.g., splice variants or allelic variants. The nucleic acid sequence of an exemplary human AKR1B10 is set forth in SEQ ID NO: 2. The amino acid sequence of an exemplary protein encoded by human AKR1B10 is shown in SEQ ID NO: 34.

The term “AKR1B15” as used herein, refers to any native AKR1B15 (Aldo-Keto Reductase Family 1, Member B15) from any vertebrate source, including mammals such as primates (e.g., humans) and rodents (e.g., mice and rats), unless otherwise indicated. The term encompasses “full-length,” unprocessed AKR1B15 as well as any form of AKR1B15 that results from processing in the cell. The term also encompasses naturally occurring variants of AKR1B15, e.g., splice variants or allelic variants. The nucleic acid sequence of an exemplary human AKR1B15 is set forth in SEQ ID NO: 3. The amino acid sequence of an exemplary protein encoded by human AKR1B15 is shown in SEQ ID NO: 35.

The term “AKR1C2” as used herein, refers to any native AKR1C2 (Aldo-Keto Reductase Family 1, Member C2) from any vertebrate source, including mammals such as primates (e.g., humans) and rodents (e.g., mice and rats), unless otherwise indicated. The term encompasses “full-length,” unprocessed AKR1C2 as well as any form of AKR1C2 that results from processing in the cell. The term also encompasses naturally occurring variants of AKR1C2, e.g., splice variants or allelic variants. The nucleic acid sequence of an exemplary human AKR1C2 is set forth in SEQ ID NO: 4. The amino acid sequence of an exemplary protein encoded by human AKR1C2 is shown in SEQ ID NO: 36.

The term “AKR1C3” as used herein, refers to any native AKR1C3 (Aldo-Keto Reductase Family 1, Member C3) from any vertebrate source, including mammals such as primates (e.g., humans) and rodents (e.g., mice and rats), unless otherwise indicated. The term encompasses “full-length,” unprocessed AKR1C3 as well as any form of AKR1C3 that results from processing in the cell. The term also encompasses naturally occurring variants of AKR1C3, e.g., splice variants or allelic variants. The nucleic acid sequence of an exemplary human AKR1C3 is set forth in SEQ ID NO: 5. The amino acid sequence of an exemplary protein encoded by human AKR1C3 is shown in SEQ ID NO: 37.

The term “AKR1C4” as used herein, refers to any native AKR1C4 (Aldo-Keto Reductase Family 1, Member C4) from any vertebrate source, including mammals such as primates (e.g., humans) and rodents (e.g., mice and rats), unless otherwise indicated. The term encompasses “full-length,” unprocessed AKR1C4 as well as any form of AKR1C4 that results from processing in the cell. The term also encompasses naturally occurring variants of AKR1C4, e.g., splice variants or allelic variants. The nucleic acid sequence of an exemplary human AKR1C4 is set forth in SEQ ID NO: 6. The amino acid sequence of an exemplary protein encoded by human AKR1C4 is shown in SEQ ID NO: 38.

The term “CABYR” as used herein, refers to any native CABYR (Calcium Binding Tyrosine-(Y)-Phosphorylation Regulated) from any vertebrate source, including mammals such as primates (e.g., humans) and rodents (e.g., mice and rats), unless otherwise indicated. The term encompasses “full-length,” unprocessed CABYR as well as any form of CABYR that results from processing in the cell. The term also encompasses naturally occurring variants of CABYR, e.g., splice variants or allelic variants. The nucleic acid sequence of an exemplary human CABYR is set forth in SEQ ID NO: 7. The amino acid sequence of an exemplary protein encoded by human CABYR is shown in SEQ ID NO: 39.

The term “CYP4F11” as used herein, refers to any native CYP4F11 (Cytochrome P450, Family 4, Subfamily F, Polypeptide 11) from any vertebrate source, including mammals such as primates (e.g., humans) and rodents (e.g., mice and rats), unless otherwise indicated. The term encompasses “full-length,” unprocessed CYP4F11 as well as any form of CYP4F11 that results from processing in the cell. The term also encompasses naturally occurring variants of CYP4F11, e.g., splice variants or allelic variants. The nucleic acid sequence of an exemplary human CYP4F11 is set forth in SEQ ID NO: 8. The amino acid sequence of an exemplary protein encoded by human CYP4F11 is shown in SEQ ID NO: 40.

The term “FECH” as used herein, refers to any native FECH (Ferrochelatase) from any vertebrate source, including mammals such as primates (e.g., humans) and rodents (e.g., mice and rats), unless otherwise indicated. The term encompasses “full-length,” unprocessed FECH as well as any form of FECH that results from processing in the cell. The term also encompasses naturally occurring variants of FECH, e.g., splice variants or allelic variants. The nucleic acid sequence of an exemplary human FECH is set forth in SEQ ID NO: 9. The amino acid sequence of an exemplary protein encoded by human FECH is shown in SEQ ID NO: 41.

The term “FTL” as used herein, refers to any native FTL (Ferritin, Light Polypeptide) from any vertebrate source, including mammals such as primates (e.g., humans) and rodents (e.g., mice and rats), unless otherwise indicated. The term encompasses “full-length,” unprocessed FTL as well as any form of FTL that results from processing in the cell. The term also encompasses naturally occurring variants of FTL, e.g., splice variants or allelic variants. The nucleic acid sequence of an exemplary human FTL is set forth in SEQ ID NO: 10. The amino acid sequence of an exemplary protein encoded by human FTL is shown in SEQ ID NO: 42.

The term “GCLM” as used herein, refers to any native GCLM (Glutamate-Cysteine Ligase, Modifier Subunit) from any vertebrate source, including mammals such as primates (e.g., humans) and rodents (e.g., mice and rats), unless otherwise indicated. The term encompasses “full-length,” unprocessed GCLM as well as any form of GCLM that results from processing in the cell. The term also encompasses naturally occurring variants of GCLM, e.g., splice variants or allelic variants. The nucleic acid sequence of an exemplary human GCLM is set forth in SEQ ID NO: 11. The amino acid sequence of an exemplary protein encoded by human GCLM is shown in SEQ ID NO: 43.

The term “GSR” as used herein, refers to any native GSR (Glutathione Reductase) from any vertebrate source, including mammals such as primates (e.g., humans) and rodents (e.g., mice and rats), unless otherwise indicated. The term encompasses “full-length,” unprocessed GSR as well as any form of GSR that results from processing in the cell. The term also encompasses naturally occurring variants of GSR, e.g., splice variants or allelic variants. The nucleic acid sequence of an exemplary human GSR is set forth in SEQ ID NO: 12. The amino acid sequence of an exemplary protein encoded by human GSR is shown in SEQ ID NO: 44.

The term “KYNU” as used herein, refers to any native KYNU (Kynureninase) from any vertebrate source, including mammals such as primates (e.g., humans) and rodents (e.g., mice and rats), unless otherwise indicated. The term encompasses “full-length,” unprocessed KYNU as well as any form of KYNU that results from processing in the cell. The term also encompasses naturally occurring variants of KYNU, e.g., splice variants or allelic variants. The nucleic acid sequence of an exemplary human KYNU is set forth in SEQ ID NO: 13. The amino acid sequence of an exemplary protein encoded by human KYNU is shown in SEQ ID NO: 45.

The term “ME1” as used herein, refers to any native ME1 (Malic Enzyme 1, NADP (+)-Dependent, Cytosolic) from any vertebrate source, including mammals such as primates (e.g., humans) and rodents (e.g., mice and rats), unless otherwise indicated. The term encompasses “full-length,” unprocessed ME1 as well as any form of ME1 that results from processing in the cell. The term also encompasses naturally occurring variants of ME1, e.g., splice variants or allelic variants. The nucleic acid sequence of an exemplary human ME1 is set forth in SEQ ID NO: 14. The amino acid sequence of an exemplary protein encoded by human ME1 is shown in SEQ ID NO: 46.

The term “NFE2L2” or “NRF2” as used herein, refers to any native NFE2L2 or NRF2 (Nuclear Factor, Erythroid 2-Like 2) from any vertebrate source, including mammals such as primates (e.g., humans) and rodents (e.g., mice and rats), unless otherwise indicated. The term encompasses “full-length,” unprocessed NFE2L2 as well as any form of NFE2L2 that results from processing in the cell. The term also encompasses naturally occurring variants of NFE2L2, e.g., splice variants or allelic variants. The nucleic acid sequence of an exemplary human NFE2L2 is set forth in SEQ ID NO: 15. The amino acid sequence of an exemplary protein encoded by human NFE2L2 is shown in SEQ ID NO: 47.

The term “NQO1” as used herein, refers to any native NQO1 (NAD (P) H Dehydrogenase, Quinone 1) from any vertebrate source, including mammals such as primates (e.g., humans) and rodents (e.g., mice and rats), unless otherwise indicated. The term encompasses “full-length,” unprocessed NQO1 as well as any form of NQO1 that results from processing in the cell. The term also encompasses naturally occurring variants of NQO1, e.g., splice variants or allelic variants. The nucleic acid sequence of an exemplary human NQO1 is set forth in SEQ ID NO: 16. The amino acid sequence of an exemplary protein encoded by human NQO1 is shown in SEQ ID NO: 48.

The term “NROB1” as used herein, refers to any native NROB1 (Nuclear Receptor Subfamily 0, Group B, Member 1) from any vertebrate source, including mammals such as primates (e.g., humans) and rodents (e.g., mice and rats), unless otherwise indicated. The term encompasses “full-length,” unprocessed NROB1 as well as any form of NROB1 that results from processing in the cell. The term also encompasses naturally occurring variants of NROB1, e.g., splice variants or allelic variants. The nucleic acid sequence of an exemplary human NROB1 is set forth in SEQ ID NO: 17. The amino acid sequence of an exemplary protein encoded by human NROB1 is shown in SEQ ID NO: 49.

The term “OSGIN1” as used herein, refers to any native OSGIN1 (Oxidative Stress Induced Growth Inhibitor 1) from any vertebrate source, including mammals such as primates (e.g., humans) and rodents (e.g., mice and rats), unless otherwise indicated. The term encompasses “full-length,” unprocessed OSGIN1 as well as any form of OSGIN1 that results from processing in the cell. The term also encompasses naturally occurring variants of OSGIN1, e.g., splice variants or allelic variants. The nucleic acid sequence of an exemplary human OSGIN1 is set forth in SEQ ID NO: 18. The amino acid sequence of an exemplary protein encoded by human OSGIN1 is shown in SEQ ID NO: 50.

The term “PGD” as used herein, refers to any native PGD (Phosphogluconate Dehydrogenase) from any vertebrate source, including mammals such as primates (e.g., humans) and rodents (e.g., mice and rats), unless otherwise indicated. The term encompasses “full-length,” unprocessed PGD as well as any form of PGD that results from processing in the cell. The term also encompasses naturally occurring variants of PGD, e.g., splice variants or allelic variants. The nucleic acid sequence of an exemplary human PGD is set forth in SEQ ID NO: 19. The amino acid sequence of an exemplary protein encoded by human PGD is shown in SEQ ID NO: 51.

The term “RSPO3” as used herein, refers to any native RSPO3 (R-Spondin 3) from any vertebrate source, including mammals such as primates (e.g., humans) and rodents (e.g., mice and rats), unless otherwise indicated. The term encompasses “full-length,” unprocessed RSPO3 as well as any form of RSPO3 that results from processing in the cell. The term also encompasses naturally occurring variants of RSPO3, e.g., splice variants or allelic variants. The nucleic acid sequence of an exemplary human RSPO3 is set forth in SEQ ID NO: 20. The amino acid sequence of an exemplary protein encoded by human RSPO3 is shown in SEQ ID NO: 52.

The term “SLC7A11” as used herein, refers to any native SLC7A11 (Solute Carrier Family 7 (Anionic Amino Acid Transporter Light Chain, Xc-System), Member 11) from any vertebrate source, including mammals such as primates (e.g., humans) and rodents (e.g., mice and rats), unless otherwise indicated. The term encompasses “full-length,” unprocessed SLC7A11 as well as any form of SLC7A11 that results from processing in the cell. The term also encompasses naturally occurring variants of SLC7A11, e.g., splice variants or allelic variants. The nucleic acid sequence of an exemplary human SLC7A11 is set forth in SEQ ID NO: 21. The amino acid sequence of an exemplary protein encoded by human SLC7A11 is shown in SEQ ID NO: 53.

The term “SRXN1” as used herein, refers to any native SRXN1 (Sulfiredoxin 1) from any vertebrate source, including mammals such as primates (e.g., humans) and rodents (e.g., mice and rats), unless otherwise indicated. The term encompasses “full-length,” unprocessed SRXN1 as well as any form of SRXN1 that results from processing in the cell. The term also encompasses naturally occurring variants of SRXN1, e.g., splice variants or allelic variants. The nucleic acid sequence of an exemplary human SRXN1 is set forth in SEQ ID NO: 22. The amino acid sequence of an exemplary protein encoded by human SRXN1 is shown in SEQ ID NO: 54.

The term “TALDO1” as used herein, refers to any native TALDO1 (Transaldolase 1) from any vertebrate source, including mammals such as primates (e.g., humans) and rodents (e.g., mice and rats), unless otherwise indicated. The term encompasses “full-length,” unprocessed TALDO1 as well as any form of TALDO1 that results from processing in the cell. The term also encompasses naturally occurring variants of TALDO1, e.g., splice variants or allelic variants. The nucleic acid sequence of an exemplary human TALDO1 is set forth in SEQ ID NO: 23. The amino acid sequence of an exemplary protein encoded by human TALDO1 is shown in SEQ ID NO: 55.

The term “TRIM16” as used herein, refers to any native TRIM16 (Tripartite Motif Containing 16) from any vertebrate source, including mammals such as primates (e.g., humans) and rodents (e.g., mice and rats), unless otherwise indicated. The term encompasses “full-length,” unprocessed TRIM16 as well as any form of TRIM16 that results from processing in the cell. The term also encompasses naturally occurring variants of TRIM16, e.g., splice variants or allelic variants. The nucleic acid sequence of an exemplary human TRIM16 is set forth in SEQ ID NO: 24. The amino acid sequence of an exemplary protein encoded by human TRIM16 is shown in SEQ ID NO: 56.

The term “TRIM16L” as used herein, refers to any native TRIM16L (Tripartite Motif Containing 16-Like) from any vertebrate source, including mammals such as primates (e.g., humans) and rodents (e.g., mice and rats), unless otherwise indicated. The term encompasses “full-length,” unprocessed TRIM16L as well as any form of TRIM16L that results from processing in the cell. The term also encompasses naturally occurring variants of TRIM16L, e.g., splice variants or allelic variants. The nucleic acid sequence of an exemplary human TRIM16L is set forth in SEQ ID NO: 25. The amino acid sequence of an exemplary protein encoded by human TRIM16L is shown in SEQ ID NO: 57.

The term “TXN” as used herein, refers to any native TXN (Thioredoxin) from any vertebrate source, including mammals such as primates (e.g., humans) and rodents (e.g., mice and rats), unless otherwise indicated. The term encompasses “full-length,” unprocessed TXN as well as any form of TXN that results from processing in the cell. The term also encompasses naturally occurring variants of TXN, e.g., splice variants or allelic variants. The nucleic acid sequence of an exemplary human TXN is set forth in SEQ ID NO: 26. The amino acid sequence of an exemplary protein encoded by human TXN is shown in SEQ ID NO: 58.

The term “TXNRD1” as used herein, refers to any native TXNRD1 (Thioredoxin Reductase 1) from any vertebrate source, including mammals such as primates (e.g., humans) and rodents (e.g., mice and rats), unless otherwise indicated. The term encompasses “full-length,” unprocessed TXNRD1 as well as any form of TXNRD1 that results from processing in the cell. The term also encompasses naturally occurring variants of TXNRD1, e.g., splice variants or allelic variants. The nucleic acid sequence of an exemplary human TXNRD1 is set forth in SEQ ID NO: 27. The amino acid sequence of an exemplary protein encoded by human TXNRD1 is shown in SEQ ID NO: 59.

The term “UGDH” as used herein, refers to any native UGDH (Uridine Diphospho (UDP)-Glucose 6-Dehydrogenase) from any vertebrate source, including mammals such as primates (e.g., humans) and rodents (e.g., mice and rats), unless otherwise indicated. The term encompasses “full-length,” unprocessed

UGDH as well as any form of UGDH that results from processing in the cell. The term also encompasses naturally occurring variants of UGDH, e.g., splice variants or allelic variants. The nucleic acid sequence of an exemplary human UGDH is set forth in SEQ ID NO: 28. The amino acid sequence of an exemplary protein encoded by human UGDH is shown in SEQ ID NO: 60.

The terms “sample” and “biological sample” are used interchangeably to refer to any biological sample obtained from an individual including body fluids, body tissue (e.g., tumor tissue), cells, or other sources. Body fluids are, e.g., lymph, sera, whole fresh blood, peripheral blood mononuclear cells, frozen whole blood, plasma (including fresh or frozen), urine, saliva, semen, synovial fluid and spinal fluid. Samples also include breast tissue, renal tissue, colonic tissue, brain tissue, muscle tissue, synovial tissue, skin, hair follicle, bone marrow, and tumor tissue. Methods for obtaining tissue biopsies and body fluids from mammals are well known in the art.

By “tissue sample” or “cell sample” is meant a collection of similar cells obtained from a tissue of a subject or individual. The source of the tissue or cell sample may be solid tissue as from a fresh, frozen and/or preserved organ, tissue sample, biopsy, and/or aspirate; blood or any blood constituents such as plasma; bodily fluids such as cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” “reference tissue,” “control sample,” “control cell,” or “control tissue,” as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual. In another embodiment, a reference sample, reference cell, reference tissue, control sample, control cell, or control tissue is obtained from one or more cell lines (e.g., one or more normal cell lines).

The phrase “identifying a patient” or “identifies a patient” as used herein, refers to using the information or data generated relating to the level of at least one of the genes set forth in Table 1, the presence of NRF2 mRNA having deletion of all or a portion of its exon 2 or exon 2+3, or the presence of NRF2 protein having a deletion of all or a portion of its Neh2 or Neh2+4 in a sample of a patient to identify or select the patient as more likely to benefit or less likely to benefit from a therapy comprising a NRF2 pathway antagonist. The information or data used or generated may be in any form, written, oral or electronic. In some embodiments, using the information or data generated includes communicating, presenting, reporting, storing, sending, transferring, supplying, transmitting, dispensing, or combinations thereof. In some embodiments, communicating, presenting, reporting, storing, sending, transferring, supplying, transmitting, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by a laboratory or medical professional. In some embodiments, the information or data includes a comparison of the level of at least one of the genes set forth in Table 1 to a reference level. In some embodiments, the information or data includes an indication that at least one of the genes set forth in Table 1 is present or absent in the sample. In some embodiments, the information or data includes an indication that the NRF2 mRNA has a deletion of all or a portion of its exon 2 or exon 2+3. In some embodiments, the information or data includes an indication that the NRF2 protein has a deletion of all or a portion of its Neh2 or Neh2+4. In some embodiments, the information or data includes an indication that the patient is more likely or less likely to respond to a therapy comprising a NRF2 pathway antagonist).

The term “primer” refers to a single-stranded polynucleotide that is capable of hybridizing to a nucleic acid and allowing polymerization of a complementary nucleic acid, generally by providing a free 3′-OH group.

As used herein, the term “treatment” (and variations thereof, such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “administering” is meant a method of giving a dosage of a compound (e.g., a NRF2 pathway antagonist) to a subject. The compositions utilized in the methods described herein can be administered, for example, intravitreally (e.g., by intravitreal injection), by eye drop, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subcutaneously, subconjunctivally, intravesicularly, mucosally, intrapericardially, intraumbilically, intraocularly, intraorbitally, orally, topically, transdermally, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated).

An “effective amount” of an agent, e.g., a pharmaceutical formulation, refers to an amount effective, at dosages and for periods of time necessary, to achieve the desired therapeutic or prophylactic result.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:
100 times the fraction X/Y
   where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “anti-neoplastic” refers to a composition useful in treating cancer comprising at least one active therapeutic agent, e.g., “anti-cancer agent.” Examples of therapeutic agents (anti-cancer agents) include, but are limited to, e.g., chemotherapeutic agents, growth inhibitory agents, cytotoxic agents, agents used in radiation therapy, anti-angiogenesis agents, apoptotic agents, anti-tubulin agents, and other-agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (TARCEVA™), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, and the like. Combinations thereof are also included in the invention.

The term “cytotoxic agent” as used herein refers to a substance that inhibits or prevents a cellular function and/or causes cell death or destruction. Cytotoxic agents include, but are not limited to, radioactive isotopes (e.g., At211, 1131, |125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes, antibiotics, toxins such as small molecule toxins or enzymatically active toxins of bacterial, fungal, plant or animal origin, including fragments and/or variants thereof, and the various antitumor or anticancer agents disclosed below.

A “chemotherapeutic agent” is a chemical compound useful in the treatment of cancer. Examples of chemotherapeutic agents include is a chemical compound useful in the treatment of cancer. Examples of chemotherapeutic agents include alkylating agents, such as, for example, temozolomide (TMZ), the imidazotetrazine derivative of the alkylating agent dacarbazine. Additional examples of chemotherapeutics agents include, e.g., paclitaxel or topotecan or pegylated liposomal doxorubicin (PLD). Other examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin; bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1l and calicheamicin omegal1 (see, e.g., Agnew, Chem. Intl. Ed. Engl., 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, III.), and TAXOTERE® docetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF that reduce cell proliferation and pharmaceutically acceptable salts, acids, or derivatives of any of the above.

The terms “Programmed Death Ligand 1” and “PD-L1” refer herein to a native sequence PD-L1 polypeptide, polypeptide variants, and fragments of a native sequence polypeptide and polypeptide variants. The PD-L1 polypeptide described herein may be that which is isolated from a variety of sources, such as from human tissue types or from another source, or prepared by recombinant or synthetic methods.

The term “PD-L1 axis binding antagonist” refers to a molecule that inhibits the interaction of a PD-L1 axis binding partner with one or more of its binding partners, so as to remove T-cell dysfunction resulting from signaling on the PD-1 signaling axis, with a result being restored or enhanced T-cell function. As used herein, a PD-L1 axis binding antagonist includes a PD-L1 binding antagonist and a PD-1 binding antagonist as well as molecules that interfere with the interaction between PD-L1 and PD-1 (e.g., a PD-L2-Fc fusion).

As used herein, a “PD-L1 binding antagonist” is a molecule that decreases, blocks, inhibits, abrogates or interferes with signal transduction resulting from the interaction of PD-L1 with either one or more of its binding partners, such as PD-1 and/or B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, PD-L1 binding antagonists include anti-PD-L1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-L1 with one or more of its binding partners, such as PD-1 and/or B7-1. In one embodiment, a PD-L1 binding antagonist reduces the negative signal mediated by or through cell surface proteins expressed on T lymphocytes, and other cells, mediated signaling through PD-L1 or PD-1 so as render a dysfunctional T-cell less dysfunctional. In some embodiments, a PD-L1 binding antagonist is an anti-PD-L1 antibody. In a specific aspect, an anti-PD-L1 antibody is YW243.55.S70. In another specific aspect, an anti-PD-L1 antibody is MDX-1105. In still another specific aspect, an anti-PD-L1 antibody is atezolizumab (MPDL3280A). In still another specific aspect, an anti-PD-L1 antibody is MEDI4736 (druvalumab). In still another specific aspect, an anti-PD-L1 antibody is MSB0010718C (avelumab).

As used herein, a “PD-1 binding antagonist” is a molecule that decreases, blocks, inhibits, abrogates or interferes with signal transduction resulting from the interaction of PD-1 with one or more of its binding partners, such as PD-L1 and/or PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies and antigen-binding fragments thereof, immunoadhesins, fusion proteins, oligopeptides, small molecule antagonists, polynucleotide antagonists, and other molecules that decrease, block, inhibit, abrogate or interfere with signal transduction resulting from the interaction of PD-1 with PD-L1 and/or PD-L2. In one embodiment, a PD-1 binding antagonist reduces the negative signal mediated by or through cell surface proteins expressed on T lymphocytes, and other cells, mediated signaling through PD-1 or PD-L1 so as render a dysfunctional T-cell less dysfunctional. In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is CT-011 (pidilizumab). In another specific aspect, a PD-1 binding antagonist is MEDI-0680 (AMP-514). In another specific aspect, a PD-1 binding antagonist is PDR001. In another specific aspect, a PD-1 binding antagonist is REGN2810 described herein. In another specific aspect, a PD-1 binding antagonist is BGB-108 described herein. In another specific aspect, a PD-1 binding antagonist is AMP-224.

The term “vascular endothelial growth factor” or “VEGF” refers to vascular endothelial growth factor. The term “VEGF” encompasses homologues and isoforms thereof. The term “VEGF” also encompasses the known isoforms, e.g., splice isoforms, of VEGF, e.g., VEGF₁₁₁, VEGF₁₂₁, VEGF₁₄₅, VEGF₁₆₅, VEGF₁₈₉, and VEGF206, together with the naturally-occurring allelic and processed forms thereof, including the 110-amino acid human vascular endothelial cell growth factor generated by plasmin cleavage of VEGF₁₆₅ as described in Ferrara Mol. Biol. Cell. 21:687 (2010), Leung et al., Science, 246:1306 (1989), and Houck et al., Mol. Endocrin., 5:1806 (1991). The term “VEGF” also refers to VEGFs from non-human species such as mouse, rat or primate. Sometimes the VEGF from a specific species are indicated by terms such as hVEGF for human VEGF, mVEGF for murine VEGF, and the like. The term “VEGF” is also used to refer to truncated forms of the polypeptide comprising amino acids 8 to 109 or 1 to 109 of the 165-amino acid human vascular endothelial cell growth factor. Reference to any such forms of VEGF may be identified in the present application, e.g., by “VEGF₁₀₉,” “VEGF (8-109),” “VEGF (1-109)” or “VEGF₁₆₅.” The amino acid positions for a “truncated” native VEGF are numbered as indicated in the native VEGF sequence. For example, amino acid position 17 (methionine) in truncated native VEGF is also position 17 (methionine) in native VEGF. The truncated native VEGF has binding affinity for the KDR and Flt-1 receptors comparable to native VEGF. The term “VEGF variant” as used herein refers to a VEGF polypeptide which includes one or more amino acid mutations in the native VEGF sequence. Optionally, the one or more amino acid mutations include amino acid substitution(s). For purposes of shorthand designation of VEGF variants described herein, it is noted that numbers refer to the amino acid residue position along the amino acid sequence of the putative native VEGF (provided in Leung et al., supra and Houck et al., supra).

The term “VEGF antagonist,” as used herein, refers to a molecule capable of binding to VEGF, reducing VEGF expression levels, or neutralizing, blocking, inhibiting, abrogating, reducing, or interfering with VEGF biological activities, including, but not limited to, VEGF binding to one or more VEGF receptors, VEGF signaling, and VEGF-mediated angiogenesis and endothelial cell survival or proliferation. For example, a molecule capable of neutralizing, blocking, inhibiting, abrogating, reducing, or interfering with VEGF biological activities can exert its effects by binding to one or more VEGF receptor (VEGFR) (e.g., VEGFR1, VEGFR2, VEGFR3, membrane-bound VEGF receptor (mbVEGFR), or soluble VEGF receptor (sVEGFR)). Included as VEGF antagonists useful in the methods of the invention are polypeptides that specifically bind to VEGF, anti-VEGF antibodies and antigen-binding fragments thereof, receptor molecules and derivatives which bind specifically to VEGF thereby sequestering its binding to one or more receptors, fusions proteins (e.g., VEGF-Trap (Regeneron)), and VEGF121-gelonin (Peregrine). VEGF antagonists also include antagonist variants of VEGF polypeptides, antisense nucleobase oligomers complementary to at least a fragment of a nucleic acid molecule encoding a VEGF polypeptide; small RNAs complementary to at least a fragment of a nucleic acid molecule encoding a VEGF polypeptide; ribozymes that target VEGF; peptibodies to VEGF; and VEGF aptamers. VEGF antagonists also include polypeptides that bind to VEGFR, anti-VEGFR antibodies, and antigen-binding fragments thereof, and derivatives which bind to VEGFR thereby blocking, inhibiting, abrogating, reducing, or interfering with VEGF biological activities (e.g., VEGF signaling), or fusions proteins. VEGF antagonists also include nonpeptide small molecules that bind to VEGF or VEGFR and are capable of blocking, inhibiting, abrogating, reducing, or interfering with VEGF biological activities. Thus, the term “VEGF activities” specifically includes VEGF-mediated biological activities of VEGF. In certain embodiments, the VEGF antagonist reduces or inhibits, by at least 10%, 20%, 30%, 40%, 50%, 60%, 70%, 80%, 90% or more, the expression level or biological activity of VEGF. In some embodiments, the VEGF inhibited by the VEGF-specific antagonist is VEGF (8-109), VEGF (1-109), or VEGF₁₆₅.

As used herein, VEGF antagonists can include, but are not limited to, anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGF antibodies (e.g., bevacizumab, sevacizumab, and ranibizumab), and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib).

The terms “anti-VEGF antibody,” an “antibody that binds to VEGF,” and “antibody that specifically binds VEGF” refer to an antibody that is capable of binding VEGF with sufficient affinity such that the antibody is useful as a diagnostic and/or therapeutic agent in targeting VEGF. In one embodiment, the extent of binding of an anti-VEGF antibody to an unrelated, non-VEGF protein is less than about 10% of the binding of the antibody to VEGF as measured, for example, by a radioimmunoassay (RIA). In certain embodiments, an antibody that binds to VEGF has a dissociation constant (Kd) of ≤1μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M). In certain embodiments, an anti-VEGF antibody binds to an epitope of VEGF that is conserved among VEGF from different species.

In certain embodiments, the anti-VEGF antibody can be used as a therapeutic agent in targeting and interfering with diseases or conditions wherein the VEGF activity is involved. Also, the antibody may be subjected to other biological activity assays, e.g., in order to evaluate its effectiveness as a therapeutic. Such assays are known in the art and depend on the target antigen and intended use for the antibody. Examples include the HUVEC inhibition assay; tumor cell growth inhibition assays (as described in WO 89/06692, for example); antibody-dependent cellular cytotoxicity (ADCC) and complement-mediated cytotoxicity (CDC) assays (U.S. Pat. No. 5,500,362); and agonistic activity or hematopoiesis assays (see WO 95/27062). An anti-VEGF antibody will usually not bind to other VEGF homologues such as VEGF-B or VEGF-C, nor other growth factors such as PIGF, PDGF, or bFGF. In one embodiment, anti-VEGF antibody is a monoclonal antibody that binds to the same epitope as the monoclonal anti-VEGF antibody A4.6.1 produced by hybridoma ATCC HB 10709. In another embodiment, the anti-VEGF antibody is a recombinant humanized anti-VEGF monoclonal antibody generated according to Presta et al. (1997) Cancer Res. 57:4593-4599, including but not limited to the antibody known as bevacizumab (BV; AVASTIN®).

The anti-VEGF antibody “ranibizumab” also known as “LUCENTIS®” or “rhuFab V2” is a humanized, affinity-matured anti-human VEGF Fab fragment. Ranibizumab is produced by standard recombinant technology methods in Escherichia coli expression vector and bacterial fermentation. Ranibizumab is not glycosylated and has a molecular mass of ˜48,000 daltons. See WO 98/45331 and US 2003/0190317. Additional preferred antibodies include the G6 or B20 series antibodies (e.g., G6-31, B20-4.1), as described in PCT Application Publication Nos. WO 2005/012359 and WO 2005/044853, which are each incorporated herein by reference in their entirety. For additional preferred antibodies see U.S. Pat. Nos. 7,060,269, 6,582,959, 6,703,020; 6,054,297; WO98/45332; WO 96/30046; WO94/10202; EP 0666868B1; U.S. Patent Application Publication Nos. 2006009360, 20050186208, 20030206899, 20030190317, 20030203409, and 20050112126; and Popkov et al., Journal of Immunological Methods 288:149-164 (2004). Other preferred antibodies include those that bind to a functional epitope on human VEGF comprising of residues F17, M18, D19, Y21, Y25, Q89, 191, K101, E103, and C104 or, alternatively, comprising residues F17, Y21, Q22, Y25, D63, 183, and Q89. Additional anti-VEGF antibodies include anti-VEGF antibodies described in PCT Application Publication No. WO 2009/155724.

The term “co-administered” is used herein to refer to administration of two or more therapeutic agents, where at least part of the administration overlaps in time. Accordingly, co-administration includes a dosing regimen when the administration of one or more agent(s) continues after discontinuing the administration of one or more other agent(s).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

### III. Methods

### A. Diagnostic Methods

Provided herein are methods for diagnosing cancer (e.g., a lung cancer (e.g., squamous NSCLC or non-squamous NSCLC) or a head and neck cancer (e.g., HNSC)) in a subject. Also provided herein are methods for identifying a subject having a cancer that is a NRF2-dependent cancer (e.g., lung cancer, e.g., squamous non-small cell lung cancer or non-squamous non-small cell lung cancer, or head and neck cancer). Any of the methods may be based on the expression level of a biomarker provided herein, for example, a splice variant of NRF2 (e.g., NRF2 mRNA or NRF2 protein), or an increased expression of one or more NRF2 target genes. Any of the methods may further include administering to the subject a NRF2 pathway antagonist. Any of the methods may further include administering an effective amount of a second therapeutic (e.g., one or more (e.g., 1, 2, 3, or 4 or more) additional NRF2 pathway antagonists or one or more (e.g., 1, 2, 3, or 4 or more) anti-cancer agents) to the subject.

The invention provides a method of diagnosing a cancer in a subject, the method comprising determining the expression level of at least one gene (e.g., 1, 2, 3, 4, 5, 6, 7, 8, 9, 10, 11, 12, 13, 14, 15, 16, 17, 18, 19, 20, 21, 22, 23, 24, 25, 26, or 27 genes) selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, and FTL in a sample obtained from the subject; and comparing the expression level of the at least one gene to a reference expression level of the at least one gene, wherein an increase in the expression level of the at least one gene in the sample relative to the reference expression level of the at least one gene identifies a subject having a cancer.

The invention further provides a method of identifying a subject having a cancer that is a NRF2-dependent cancer, the method comprising determining the expression level of at least one gene (e.g., 1, 2, 3, 4, 5, 6, 7, 8, 9, 10, 11, 12, 13, 14, 15, 16, 17, 18, 19, 20, 21, 22, 23, 24, 25, 26, or 27 genes) selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, and FTL in a sample obtained from the subject; comparing the expression level of the at least one gene to a reference expression level of the at least one gene; and determining if the subject's cancer is a NRF2-dependent cancer, wherein an increase in the expression level of the at least one gene in the sample relative to the reference expression level of the at least one gene identifies a subject having a NRF2-dependent cancer.

In any of the preceding methods, the expression level of one or more (e.g., 1, 2, 3, 4, 5, 6, 7, 8, 9, 10, 11, 12, 13, 14, 15, 16, 17, 18, 19, 20, or 21) of AKR1B10, AKR1C2, SRXN1, OSGIN1, FECH, GCLM,

TRIM16, KYNU, CABYR, SLC7A11, TRIM16L, AKR1C4, NROB1, UGDH, TXNRD1, GSR, AKR1C3, TALDO1, PGD, TXN, or NQO1 is determined.

In any of the preceding methods, the expression level of one or more (e.g., 1, 2, 3, 4, 5, 6, 7, 8, 9, 10, 11, or 12) newly identified NRF2 target genes is determined. Newly identified NRF2 target genes include AKR1B10, AKR1C2, ME1, KYNU, CABYR, TRIM16L, AKR1C4, CYP4F11, RSPO3, AKR1B15, NROB1, and AKR1C3.

The invention further provides a method of diagnosing a cancer in a subject, the method comprising determining the mRNA expression level of NRF2 comprising a deletion in all or a portion of its exon 2 in a sample obtained from the subject (e.g., a tumor sample), wherein the presence of NRF2 comprising a deletion in all or a portion of its exon 2 identifies the subject as having a cancer. In some embodiments, the NRF2 further comprises a deletion in all or a portion of its exon 3. Presence and/or expression levels of a gene (e.g., NRF2, KEAP1, AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, or FTL) may be determined qualitatively or quantitatively based on any suitable criterion known in the art, including, but not limited to DNA, mRNA, cDNA, protein fragments, and/or gene copy number.

The invention further provides a method of diagnosing a cancer in a subject, the method comprising determining the protein expression level of NRF2 comprising a deletion in all or a portion of its Neh2 domain in a sample obtained from the subject, wherein the presence of NRF2 comprising a deletion in all or a portion of its Neh2 domain identifies the subject as having a cancer. In some embodiments, the NRF2 further comprises a deletion in all or a portion of its Neh4 domain.

The invention further provides a method of identifying a subject having cancer, the method comprising determining the mRNA expression level of NRF2 comprising a deletion in all or a portion of its exon 2 in a sample obtained from the subject (e.g., a tumor sample), wherein the presence of NRF2 comprising a deletion in all or a portion of its exon 2 identifies the subject as having a cancer. In some embodiments, the NRF2 further comprises a deletion in all or a portion of its exon 3. Presence and/or expression levels of a gene (e.g., NRF2, KEAP1, AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, or FTL) may be determined qualitatively or quantitatively based on any suitable criterion known in the art, including, but not limited to DNA, RNA, cDNA, protein fragments, and/or gene copy number.

The invention further provides a method of identifying a subject having cancer, the method comprising determining the protein expression level of NRF2 comprising a deletion in all or a portion of its Neh2 domain in a sample obtained from the subject, wherein the presence of NRF2 comprising a deletion in all or a portion of its Neh2 domain identifies the subject as having a cancer. In some embodiments, the NRF2 further comprises a deletion in all or a portion of its Neh4 domain.

The presence and/or expression level/amount of various biomarkers described herein in a sample can be analyzed by a number of methodologies, many of which are known in the art and understood by the skilled artisan, including, but not limited to, immunohistochemistry (“IHC”), Western blot analysis, immunoprecipitation, molecular binding assays, ELISA, ELIFA, fluorescence activated cell sorting (“FACS”), MassARRAY, proteomics, quantitative blood based assays (e.g., Serum ELISA), biochemical enzymatic activity assays, in situ hybridization, fluorescence in situ hybridization (FISH), Southern analysis, Northern analysis, whole genome sequencing, massively parallel DNA sequencing (e.g., next-generation sequencing), NANOSTRING®, polymerase chain reaction (PCR) including quantitative real time PCR (qRT-PCR) and other amplification type detection methods, such as, for example, branched DNA, SISBA, TMA and the like, RNA-seq, microarray analysis, gene expression profiling, and/or serial analysis of gene expression (“SAGE”), as well as any one of the wide variety of assays that can be performed by protein, gene, and/or tissue array analysis. Typical protocols for evaluating the status of genes and gene products are found, for example in Ausubel et al., eds., 1995, Current Protocols In Molecular Biology, Units 2 (Northern Blotting), 4 (Southern Blotting), 15 (Immunoblotting) and 18 (PCR Analysis). Multiplexed immunoassays such as those available from Rules Based Medicine or Meso Scale Discovery (“MSD”) may also be used.

In some embodiments of any of the methods described herein, DNA from clinical tumor samples can be sequenced using a next-generation sequencing method, such as the targeted gene pulldown and sequencing method described in Frampton et al. (Nature Biotechnology. 31 (11): 1023-1033, 2013), which is incorporated by reference herein in its entirety. Such a next-generation sequencing method can be used with any of the methods disclosed herein to detect various mutations (e.g., insertions, deletions, base substitutions, focal gene amplifications, and/or homozygous gene deletions), while enabling the use of small samples (e.g., from small-core needle biopsies, fine-needle aspirations, and/or cell blocks) or fixed samples (e.g., formalin-fixed and paraffin-embedded (FFPE) samples).

In any of the preceding methods, the presence and/or expression level/amount of a biomarker (e.g., NRF2, KEAP1, AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, or FTL) is measured by determining protein expression levels of the biomarker. In certain embodiments, the method comprises contacting the biological sample with antibodies that specifically bind to a biomarker (e.g., anti-NRF2 antibodies) under conditions permissive for binding of the biomarker, and detecting whether a complex is formed between the antibodies and biomarker. Such method may be an in vitro or in vivo method. Any method of measuring protein expression levels known in the art or provided herein may be used. For example, in some embodiments, a protein expression level of a biomarker is determined using a method selected from the group consisting of flow cytometry (e.g., fluorescence-activated cell sorting (FACS™)), Western blot, enzyme-linked immunosorbent assay (ELISA), immunoprecipitation, immunohistochemistry (IHC), immunofluorescence, radioimmunoassay, dot blotting, immunodetection methods, HPLC, surface plasmon resonance, optical spectroscopy, mass spectrometry, and HPLC. In some embodiments, the protein expression level of the biomarker is determined in tumor cells.

In some embodiments, the presence and/or expression level/amount of a biomarker (e.g., NRF2,KEAP1, AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, or FTL) is measure by determining mRNA expression levels of the biomarker. In certain embodiments, presence and/or expression level/amount of a gene is determined using a method comprising: (a) performing gene expression profiling, PCR (such as RT-PCR), RNA-seq, microarray analysis, SAGE, MassARRAY technique, or FISH on a sample (such as a subject cancer sample); and b) determining presence and/or expression level/amount of a biomarker in the sample. In one embodiment, the PCR method is qRT-PCR. In one embodiment, the PCR method is multiplex-PCR. In some embodiments, gene expression is measured by microarray. In some embodiments, gene expression is measured by qRT-PCR. In some embodiments, expression is measured by multiplex-PCR.

Methods for the evaluation of mRNAs in cells are well known and include, for example, hybridization assays using complementary DNA probes (such as in situ hybridization using labeled riboprobes specific for the one or more genes, Northern blot and related techniques) and various nucleic acid amplification assays (such as RT-PCR using complementary primers specific for one or more of the genes, and other amplification type detection methods, such as, for example, branched DNA, SISBA, TMA and the like). Samples from mammals can be conveniently assayed for mRNAs using Northern, dot blot, or PCR analysis. In addition, such methods can include one or more steps that allow one to determine the levels of target mRNA in a biological sample (e.g., by simultaneously examining the levels a comparative control mRNA sequence of a “housekeeping” gene such as an actin family member).

In some embodiments of any of the methods, the biomarker is NRF2 (e.g., exon 2-deleted NRF2 or exon 2+3-deleted NRF2). In one embodiment, expression level of biomarker is determined using a method comprising performing WGS analysis on a sample (such as a tumor sample obtained from a patient) and determining expression level of a biomarker in the sample. In some embodiments, presence of exon 2-deleted NRF2 or exon 2+3-deleted NRF2 is determined relative to a reference. In some embodiments, the reference is a reference value. In some embodiments, the reference is a reference sample (e.g., a control cell line sample, a tissue sample from non-cancerous patient, or a wild-type NRF2 tissue sample).

Additionally or alternatively to mRNA expression analysis, other biomarkers, such as protein expression, may be quantified according to methods described above. For example, methods of the invention include testing a sample for a genomic biomarker (e.g., the presence of exon 2-deleted NRF2 or exon 2+3-deleted NRF2, or the upregulation of one or more NRF2 target genes, e.g., AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, or FTL) and additionally testing a sample for a protein biomarker (e.g., protein transcripts of one or more of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NROB1, UGDH, TXNRD1, GSR, AKR1C3, TALDO1, PGD, TXN, NQO1, or FTL).

In some embodiments of any of the methods, a DNA sequence may serve as a biomarker. DNA can be quantified according to any method known in the art, including, but not limited to, PCR, exome-seq (e.g., whole exome sequencing), DNA microarray analysis, NANOSTRING®, or whole genome sequencing.

In some instances, the expression level of the genes in the sample is an average (e.g., mean expression or median expression) of the genes, the reference expression level of the genes is an average (e.g., mean expression or median expression) of the genes of the reference, and the average of the genes of the sample is compared to the average of the genes of the reference.

In certain embodiments, the presence and/or expression levels/amount of a biomarker in a first sample is increased or elevated as compared to presence/absence and/or expression levels/amount in a second sample. In certain embodiments, the presence/absence and/or expression levels/amount of a biomarker in a first sample is decreased or reduced as compared to presence and/or expression levels/amount in a second sample. In certain embodiments, the second sample is a reference sample, reference cell, reference tissue, control sample, control cell, or control tissue. Additional disclosures for determining the presence/absence and/or expression levels/amount of a gene are described herein.

In certain embodiments, a reference sample, reference cell, reference tissue, control sample, control cell, or control tissue is a single sample or combined multiple samples from the same subject or individual that are obtained at one or more different time points than when the test sample is obtained. For example, a reference sample, reference cell, reference tissue, control sample, control cell, or control tissue is obtained at an earlier time point from the same subject or individual than when the test sample is obtained. Such reference sample, reference cell, reference tissue, control sample, control cell, or control tissue may be useful if the reference sample is obtained during initial diagnosis of cancer and the test sample is later obtained when the cancer becomes metastatic.

In certain embodiments, a reference sample, reference cell, reference tissue, control sample, control cell, or control tissue is a combined multiple samples from one or more healthy individuals who are not the patient. In certain embodiments, a reference sample, reference cell, reference tissue, control sample, control cell, or control tissue is a combined multiple samples from one or more individuals with a disease or disorder (e.g., cancer) who are not the subject or individual. In certain embodiments, a reference sample, reference cell, reference tissue, control sample, control cell, or control tissue is pooled RNA samples from normal tissues or pooled plasma or serum samples from one or more individuals who are not the patient. In certain embodiments, a reference sample, reference cell, reference tissue, control sample, control cell, or control tissue is pooled RNA samples from tumor tissues or pooled plasma or serum samples from one or more individuals with a disease or disorder (e.g., cancer) who are not the patient.

In some embodiments of any of the methods, elevated or increased expression refers to an overall increase of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene (DNA or mRNA))), detected by standard art-known methods such as those described herein, as compared to a reference sample, reference cell, reference tissue, control sample, control cell, or control tissue. In certain embodiments, the elevated expression refers to the increase in expression level/amount of a biomarker in the sample wherein the increase is at least about any of 1.5×, 1.75×, 2×, 3×, 4×, 5×, 6×, 7×, 8×, 9×, 10×, 25×, 50×, 75×, or 100× the expression level/amount of the respective biomarker in a reference sample, reference cell, reference tissue, control sample, control cell, or control tissue. In some embodiments, elevated expression refers to an overall increase of greater than about 1.5 fold, about 1.75 fold, about 2 fold, about 2.25 fold, about 2.5 fold, about 2.75 fold, about 3.0 fold, or about 3.25 fold as compared to a reference sample, reference cell, reference tissue, control sample, control cell, control tissue, or internal control (e.g., housekeeping gene).

In some embodiments of any of the methods, reduced expression refers to an overall reduction of about any of 10%, 20%, 30%, 40%, 50%, 60%, 70%, 80%, 90%, 95%, 96%, 97%, 98%, 99% or greater, in the level of biomarker (e.g., protein or nucleic acid (e.g., gene (DNA or mRNA))), detected by standard art known methods such as those described herein, as compared to a reference sample, reference cell, reference tissue, control sample, control cell, or control tissue. In certain embodiments, reduced expression refers to the decrease in expression level/amount of a biomarker in the sample wherein the decrease is at least about any of 0.9×, 0.8×, 0.7×, 0.6×, 0.5×, 0.4×, 0.3×, 0.2×, 0.1×, 0.05×, or 0.01× the expression level/amount of the respective biomarker in a reference sample, reference cell, reference tissue, control sample, control cell, or control tissue.

### B. Therapeutic Methods

The present invention provides methods for treating a patient suffering from a cancer (e.g., a lung cancer (e.g., squamous NSCLC or non-squamous NSCLC) or a head and neck cancer (e.g., HNSC)). In some instances, the methods of the invention include administering to the patient an effective amount of a NRF2 pathway antagonist. Any of the NRF2 pathway antagonists described herein or otherwise known in the art may be used in the methods. In some instances, the methods involve determining the presence and/or expression level of a NRF2 splice variant (e.g., exon 2-deleted NRF2 or exon 2+3-deleted NRF2) or a NRF2 target gene in a sample obtained from a patient and administering an NRF2 pathway antagonist to the patient based on the presence and/or expression level of a NRF2 splice variant (e.g., exon 2-deleted NRF2 or exon 2+3-deleted NRF2) or a NRF2 target gene, e.g., using any of the methods described herein, in the Examples below, or known in the art.

The invention provides a method of treating a subject suffering from a cancer (e.g., a lung cancer (e.g., squamous NSCLC or non-squamous NSCLC) or a head and neck cancer (e.g., HNSC)), the method comprising determining the expression level of at least one gene (e.g., 1, 2, 3, 4, 5, 6, 7, 8, 9, 10, 11, 12, 13, 14, 15, 16, 17, 18, 19, 20, 21, 22, 23, 24, 25, 26, or 27 genes) selected from the group consisting of AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, and FTL in a sample obtained from the subject; and comparing the expression level of the at least one gene to a reference expression level of the at least one gene, wherein an increase in the expression level of the at least one gene in the sample relative to the reference expression level of the at least one gene identifies a subject having a cancer, and administering to the subject a therapeutically effective amount of one or more NRF2 pathway antagonists.

The invention further provides a method of treating a subject suffering from a cancer (e.g., lung cancer (e.g., squamous NSCLC or non-squamous NSCLC) or head and neck cancer), wherein the expression level of one or more (e.g., 1, 2, 3, 4, 5, 6, 7, 8, 9, 10, 11, or 12) newly identified NRF2 target genes is determined. Newly identified NRF2 target genes include AKR1B10, AKR1C2, ME1, KYNU, CABYR, TRIM16L, AKR1C4, CYP4F11, RSPO3, AKR1B15, NR0B1, and AKR1C3.

In some instances, the invention further provides a method of treating a subject suffering from a cancer (e.g., lung cancer (e.g., squamous NSCLC or non-squamous NSCLC) or head and neck cancer), wherein the mRNA expression level of NRF2 comprises a deletion in all, or a portion of, its exon 2 in a sample obtained from the subject, and wherein the presence of NRF2 comprising a deletion in all or a portion of its exon 2 identifies the subject as having a cancer; and administering to the subject a therapeutically effective amount of one or more NRF2 pathway antagonists. In some embodiments, the NRF2 further comprises a deletion in all, or a portion of, its exon 3.

In some instances, the invention further provides a method of treating a subject suffering from a cancer (e.g., a lung cancer (e.g., squamous NSCLC or non-squamous NSCLC) or a head and neck cancer (e.g., HNSC)), wherein the NRF2 protein comprises a deletion in all, or a portion of, its Neh2 domain in a sample obtained from the subject, and wherein the presence of NRF2 comprising a deletion in all, or a portion of, its Neh2 domain identifies the subject as having a cancer; and administering to the subject a therapeutically effective amount of one or more NRF2 pathway antagonists. In some embodiments, the NRF2 further comprises a deletion in all or a portion of its Neh4 domain.

In any of the preceding methods, the NRF2 pathway antagonist may be any NRF2 pathway antagonist known in the art or described herein.

In some instances, the method further includes administering to the subject an effective amount of a second therapeutic agent (e.g., one or more anti-cancer agents). In some instances, the second therapeutic agent is selected from the group consisting of an anti-angiogenic agent, a chemotherapeutic agent, a growth inhibitory agent, a cytotoxic agent, an immunotherapy, and combinations thereof. In some embodiments, the immunotherapy is a VEGF antagonist (e.g., anti-VEGFR2 antibodies and related molecules (e.g., ramucirumab, tanibirumab, aflibercept), anti-VEGFR1 antibodies and related molecules (e.g., icrucumab, aflibercept (VEGF Trap-Eye; EYLEA®), and ziv-aflibercept (VEGF Trap; ZALTRAP®)), bispecific VEGF antibodies (e.g., MP-0250, vanucizumab (VEGF-ANG2), and bispecific antibodies disclosed in US 2001/0236388), bispecific antibodies including combinations of two of anti-VEGF, anti-VEGFR1, and anti-VEGFR2 arms, anti-VEGF antibodies (e.g., bevacizumab, sevacizumab, and ranibizumab), and nonpeptide small molecule VEGF antagonists (e.g., pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, and tivozanib)). In other embodiments, the immunotherapy is a PD-1 axis binding antagonist (e.g., YW243.55.S70, MDX-1105, MPDL3280A (atezolizumab), MEDI4736 (druvalumab), MSB0010718C (avelumab), MDX-1106 (nivolumab), MK-3475 (pembrolizumab), CT-011 (pidilizumab), MEDI-0680 (AMP-514), PDR001, REGN2810, BGB-108 or AMP-224).

The compositions used in the methods described herein (e.g., NRF2 pathway antagonists) can be administered by any suitable method, including, for example, intravenously, intramuscularly, subcutaneously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumorally, peritoneally, subconjunctivally, intravesicularly, mucosally, intrapericardially, intraumbilically, intraocularly, intraorbitally, orally, topically, transdermally, intravitreally (e.g., by intravitreal injection), by eye drop, by inhalation, by injection, by implantation, by infusion, by continuous infusion, by localized perfusion bathing target cells directly, by catheter, by lavage, in cremes, or in lipid compositions. The compositions utilized in the methods described herein can also be administered systemically or locally. The method of administration can vary depending on various factors (e.g., the compound or composition being administered and the severity of the condition, disease, or disorder being treated). In some embodiments, the NRF2 pathway antagonist is administered intravenously, intramuscularly, subcutaneously, topically, orally, transdermally, intraperitoneally, intraorbitally, by implantation, by inhalation, intrathecally, intraventricularly, or intranasally. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

NRF2 pathway antagonists described herein (and any additional anti-cancer agents) may be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The NRF2 pathway antagonist need not be, but is optionally formulated with and/or administered concurrently with one or more agents currently used to prevent or treat the disorder in question. The effective amount of such other agents depends on the amount of the Nrd2 pathway inhibitor present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

In some embodiments, the methods further involve administering to the patient an effective amount of a second therapeutic agent (e.g., one or more anti-cancer agents). In some embodiments, the second therapeutic agent is selected from the group consisting of an anti-angiogenic agent, a chemotherapeutic agent, a growth inhibitory agent, a cytotoxic agent, an immunotherapy, and combinations thereof.

Such combination therapies noted above encompass combined administration (where two or more therapeutic agents (e.g., a NRF2 pathway antagonist and an anti-cancer agent) are included in the same or separate formulations), and separate administration, in which case, administration of a NRF2 pathway antagonist can occur prior to, simultaneously, and/or following, administration of the additional anti-cancer agent or agents. In one embodiment, administration of NRF2 pathway antagonist and administration of an additional anti-cancer agent occur within about one month, or within about one, two or three weeks, or within about one, two, three, four, five, or six days, of each other.

c. NRF2 Pathway Antagonists for Use in the Methods of the Invention

Provided herein are methods for treating or delaying progression of a cancer (e.g., a lung cancer (e.g., squamous NSCLC) or head and neck cancer) in a subject comprising administering to the subject a therapeutically effective amount of a NRF2 pathway antagonist. Any of the preceding methods may be based on the expression level of a biomarker provided herein, for example, NRF2 expression or expression of any protein or mRNA involved in a NRF2 pathway in a tumor sample, e.g., a biopsy containing tumor cells.

In some embodiments, a NRF2 pathway antagonist is a small molecule, e.g., a small molecule capable of binding to NRF2 or protein or gene that regulates the expression, stability, or activity of NRF2.

In some embodiments, the NRF2 pathway antagonist is an antagonist of a NRF2 agonist. Examples of NRF2 agonists include, but are not limited to, cAMP response element-binding protein (CREB), CREB Binding Protein (CBP), Maf, activating transcription factor 4 (ATF4), protein kinase C (PKC), Jun, glucocorticoid receptor, UbcM2, and homologous to the E6-AP carboxyl terminus domain and Ankyrin repeat containing E3 ubiquitin protein ligase 1 (HACE1). Therefore, examples of NRF2 pathway antagonists include, but are not limited to, CREB antagonists, CBP antagonists, Maf antagonists, ATF4 antagonists, PKC antagonists, Jun antagonists, glucocorticoid receptor antagonists, UbcM2 antagonists, and HACE1 antagonists, such as those set forth in Table 2.

In some embodiments, the NRF2 pathway antagonist is an agonist of a NRF2 antagonist. Examples of NRF2 antagonists include, but are not limited to, c-Myc, SUMO, KEAP1, CUL3, retinoic acid receptor α (RARα). Therefore, examples of NRF2 pathway antagonists include, but are not limited to, c-Myc agonists, SUMO, KEAP1 agonists, CUL3 agonists, and RARα agonists, such as those set forth in Table 3.

TABLE 2

| Compound | Target |
|---|---|
| KG-501 | CREB |
| 2-naphthol-AS-E-phosphate | |
| C646 | CBP |
| 4-[4-[[5-(4,5-Dimethyl-2-nitrophenyl)-2- | |
| furanyl]methylene]-4,5-dihydro-3-methyl-5-oxo-1H- | |
| pyrazol-1-yl]benzoic acid | |
| CBP30 | CBP |
| 8-(3-chloro-4-methoxy-phenethyl)-4-(3,5- | |
| dimethyl-isoxazol-4-yl)-9-(2-(morpholin-4-yl)-propyl)- | |
| 7,9-diaza-bicyclo[4.3.0]nona-1(6),2,4,7-tetraene | |
| nivalenol | c-maf |
| 3,4,7,15-Tetrahydroxy-12,13-epoxytrichothec-9-en-8-on | |
| tomatidine | ATF4 |
| (3β,5α,22β,25S)-spirosolan-3-ol | |
| ruboxistaurin | PKC |
| (9S)-9-[(dimethylamino)methyl]-6,7,10,11- | |
| tetrahydro-9H,18H-5,21:12,17- | |
| di(metheno)dibenzo[e,k]pyrrolo[3,4- | |
| h][1,4,13]oxadiazacyclohexadecine-18,20-dione | |
| SP600125 | Jun |
| 1,9-Pyrazoloanthrone | |
| mifepristone | Glucocorticoid |
| (11β,17β)-11-[4-(Dimethylamino)phenyl]-17- | receptor |
| hydroxy-17-(1-propynyl)-estra-4,9-dien- | |
| 3-one | |
| CORT 108297 | Glucocorticoid |
| 1H-Pyrazolo[3,4-g]isoquinoline, 4a-(ethoxymethyl)- | receptor |
| 1-(4-fluorophenyl)-4,4a,5,6,7,8-hexahydro-6-[[4- | |
| (trifluoromethyl)phenyl]sulfonyl]-, (4aR)- | |

TABLE 3

| Compound | Target |
|---|---|
| AI-1 | KEAP1 |
| 4-Chloro-1,2-dihydro-1-methyl-2-oxo-3- | |
| quinolinecarboxylic acid ethyl ester, Ethyl 4-chloro-1- | |
| methyl-2-oxo-1,2-dihydroquinoline-3-carboxylate | |
| retinoic acid | RARα |
| 3,7-Dimethyl-9-(2,6,6-trimethyl-1-cyclohexen-1-yl)- | |
| 2E,4E,6E,8E,-nonatetraenoic acid | |
| CD437 | RARα |
| 6-(4-Hydroxy-3-tricyclo[3.3.1.13,7]dec-1-ylphenyl)-2- | |
| naphthalenecarboxylic acid | |
| TTNPB | RARα |
| 4-[(E)-2-(5,6,7,8-Tetrahydro-5,5,8,8-tetramethyl-2- | |
| naphthalenyl)-1-propenyl]benzoic acid | |

In some embodiments of the invention, derivatives of the compounds listed in Table 2 or 3 may also be administered as NRF2 pathway antagonists. A derivative of a compound listed in Table 2 or 3 is a small molecule that differs in structure from the parent compound, but retains the ability to antagonize a NRF2 pathway. A derivative of a compound may change its interaction with certain other molecules or proteins relative to the parent compound. A derivative of a compound may also include a salt, an adduct, or other variant of the parent compound. In some embodiments of the invention, any derivative of a compound described herein (e.g., any one compound of the compounds listed in Table 2 or 3 may be used instead of the parent compound. In some embodiments, any derivative of a compound listed in Table 2 or 3 may be used in a method of treating a subject having cancer, such as lung cancer.

In some embodiments, a NRF2 pathway antagonist is an antibody (e.g., an anti-NRF2 antibody or an antibody directed against a protein or gene that regulates NRF2 expression, stability, or activity, e.g., a target listed in Table 2 or 3). In some embodiments, the anti-NRF2 antibody is capable of inhibiting binding between NRF2 and antioxidant response element. In some embodiments, the anti-NRF2 antibody is capable of inhibiting binding between NRF2 and a cofactor (e.g., Maf, PKC, Jun, ATF4, or CBP). In some embodiments, the antibody of the invention is an antibody fragment selected from the group consisting of Fab, Fab′-SH, Fv, scFv, and (Fab′)₂ fragments. In some embodiments, the antibody is a humanized antibody. In some embodiments, the antibody is a human antibody. In some embodiments, the antibody is a derivative of a known antibody having any of the above-mentioned properties. Derivatives of antibodies include antibody variants having about 99%, 98%, 97%, 96%, 95%, 94%, 93%, 92%, 91%, 90%, 85%, 80% or lower sequence identity to its parent antibody. Percent (%) amino acid sequence identity is determined according to methods known in the art, including by ALIGN-2, as described above. In some embodiments, a NRF2 pathway antagonist includes an inhibitor of any downstream biomarker (e.g., gene or protein, e.g., a gene or protein involved in iron sequestration (e.g., Ferritin, Light Polypeptide (FTL), Ferritin, Heavy Polypeptide 1 (FTH), or Heme Oxygenase 1 (HMOX1)), GSH utilization (e.g., Glutathione Peroxidase 2 (GPX2), Glutathione S-Transferase Alpha 1 (GSTA1), Glutathione S-Transferase Alpha 2 (GSTA2), Glutathione S-Transferase Alpha 3 (GSTA3), Glutathione S-Transferase Alpha 5 (GSTA5), Glutathione S-Transferase Mu 1 (GSTM1), Glutathione S-Transferase Mu 2 (GSTM2), Glutathione S-Transferase Mu 3 (GSTM3), or Glutathione S-Transferase Pi 1 (GSTP1)), quinine detoxification (e.g., NAD (P) H Dehydrogenase, Quinone 1 (NQO1)), GSH production and regeneration (e.g., Glutamate-Cysteine Ligase, Modifier Subunit (GCLM), Glutamate-Cysteine Ligase, Catalytic Subunit (GCLC), Glutathione Reductase (GSR), or Solute Carrier Family 7 (Anionic Amino Acid Transporter Light Chain, Xc-System), Member 11 (SLC7A11, or XCT)), thioredoxin (TXN) production, regeneration, and utilization (e.g., Thioredoxin 1, (TXN1), Thioredoxin Reductase 1 (TXNRD1), or Peroxiredoxin 1 (PRDX1)), NADPH production (e.g., Glucose-6-Phosphate Dehydrogenase (G6PD), Phosphogluconate Dehydrogenase (PGD), Malic Enzyme 1, NADP(+)-Dependent, Cytosolic (ME1), Isocitrate Dehydrogenase 1 (NADP+), Soluble (IDH1)) or any of the genes or proteins thereof of Table 1).

In some embodiments, a NRF2 pathway antagonist includes a compound that inhibits NRF2 from binding to antioxidant response element (ARE) (e.g., by competitively binding to the ARE binding site on NRF2, by competitively binding to ARE, or by otherwise interfering with a transcriptional cofactor (e.g., small Maf proteins).

In some embodiments, a NRF2 pathway antagonist includes an agonist or antagonist of NRF2-related genes, such that the pharmacological effect of compound involves the downregulation of one or more pathways downstream of NRF2-mediated transcription. Such NRF2-related genes include, e.g., Kelch-Like ECH-Associated Protein 1 (KEAP1), Ectodermal-Neural Cortex 1 (With BTB Domain) (ENC1), Protein Kinase C, Delta (PRKCD), Protein Kinase C, Beta (PRKCB), Polyamine-Modulated Factor 1 (PMF1), Cullin 3(CUL3), Nuclear Factor, Erythroid 2 (NFE2), Activating Transcription Factor 4 (ATF4), Heme Oxygenase 1 (HMOX1), Heme Oxygenase 2 (HMOX2), Ubiquitin C (UBC), V-Maf Avian Musculoaponeurotic Fibrosarcoma Oncogene Homolog K (MAFK), UDP Glucuronosyltransferase 1 Family, Polypeptide A6 (UGT1A6), V-Maf Avian Musculoaponeurotic Fibrosarcoma Oncogene Homolog F (MAFF), CREB Binding Protein (CREBBP), V-Maf Avian Musculoaponeurotic Fibrosarcoma Oncogene Homolog G (MAFG), CAMP Responsive Element Binding Protein 1 (CREB1), FXYD Domain Containing lon Transport Regulator 2 (FXYD2), Jun Proto-Oncogene (JUN), Small Ubiquitin-Like Modifier 2 (SUMO2), Small Ubiquitin-Like Modifier 1 (SUMO1), V-Myc Avian Myelocytomatosis Viral Oncogene Homolog (MYC), Crystallin, Zeta (Quinone Reductase) (CRYZ), Aldo-Keto Reductase Family 7, Member A2 (Aflatoxin Aldehyde Reductase) (AKR7A2), and Glutathione S-Transferase Alpha 2 (GSTA2).

In some embodiments, a method of increasing ubiquitination of NRF2 in a cell is provided, the method comprising contacting the cell with an inhibitor of a NRF2 pathway under conditions allowing inhibition of a NRF2 pathway in a cell. Increased ubiquitination of NRF2 can be determined, e.g., by immunoaffinity enrichment of ubiquitinated NRF2 following trypsin digestion, followed by mass spectrometry, according to known methods. In some embodiments, an increase in ubiquitation may be determined by comparing the ubiquitination of a wild-type NRF2 in a cell or population of cells contacted with a NRF2 pathway antagonist with the ubiquitination of an exon 2 or exon 2+3 deleted NRF2 in a cell or a population of cells contacted with a NRF2 pathway antagonist and/or the ubiquitination of an exon 2 or exon 2+3 deleted NRF2 in a cell or a population of cells not contacted with a NRF2 pathway antagonist.

In some embodiments of the invention, the NRF2 pathway antagonist is ascorbic acid, brusatol, luteolin, or ochratoxin A.

### EXAMPLES

### Example 1

### Materials and Experimental Methods

### A. Mutation and Copy Number Analysis

For 99 NSCLC cell lines, non-synonymous mutations and copy number data for KRas, LKB1, KEAP1, and NRF2 were obtained from Klijn et al. (Nat Biotechnol. 33 (3): 306-312, 2015). Thirteen additional NSCLC cell lines were subjected to copy number analysis. In addition, exome sequencing was applied to 104 NSCLC cell lines. For the cancer genome atlas (TCGA) tumors mutation and copy number data were retrieved from cBioPortal using the R software package CGDS-R (Cerami et al. Cancer Discovery. 2:401-404, 2012; Gao et al. Sci. Signal. 6:11, 2013).

### B. RNA-seq Analysis and Derivation of a Mutant KEAP1 Gene Expression Signature

Raw RNA-seq data for 99 NSCLC cell lines were retrieved from the European Genome-phenome Archive (accession number EGAS00001000610) (PMID: 25485619). Mutations in KEAP1 and NRF2 in each of the NSCLC cell lines are provided in Table 4. Raw RNA-seq data were downloaded from TCGA and aligned to the human reference genome (GRCh37/hgl9) using GSNAP version 2013 Oct. 10 (Wu and Nacu. Bioinformatics 26:873-881, 2010), allowing maximum of 2 mismatches (parameters: “-M 2-n 10-B 2-i 1-N 1-w 200000-E 1--pairmax-rna=200000”). Gene expression levels were quantified with RPKM (reads per kilobase of target and million reads sequenced) values derived from the number of reads mapped to each RefSeq gene. Using the DESeq R package (PMID: 20979621) differential gene expression was measured between KEAP1 mutant and KEAP1 wild-type cell lines, reported as fold-change and associated adjusted p-values. For ward clustering of samples and genes (using Euclidean distance) in variance stabilized count data were used. The ‘NMF’ R package was used to create associated heatmaps.

TABLE 4

| Name | Sample Type | Gene | Mutation |
|---|---|---|---|
| BEN | Carcinoma | KEAP1 | A556T |
| NCI-H460 | Carcinoma Large Cell | KEAP1 | D236H |
| NCI-H838 | Carcinoma Non-Small Cell | KEAP1 | E444* |
| HCC44 | Carcinoma Non-Small Cell | KEAP1 | F211C |
| A549 | Carcinoma | KEAP1 | G333C |
| HCC-15 | Carcinoma Non-Small Cell | KEAP1 | G364C |
| NCI-H1648 | Adenocarcinoma | KEAP1 | G364C |
| NCI-H2110 | Carcinoma Non-Small Cell | KEAP1 | G429C |
| LXF-289 | Adenocarcinoma | KEAP1 | G430V |
| NCI-H647 | Carcinoma Non-Small Cell | KEAP1 | G523W |
| NCI-H920 | Carcinoma Non-Small Cell | KEAP1 | G603V |
| HCC4019 | Adenocarcinoma | KEAP1 | K131* |
| NCI-H23 | Carcinoma Non-Small Cell | KEAP1 | Q193H |
| NCI-H1355 | Adenocarcinoma | KEAP1 | Q75* |
| NCI-H1915 | Carcinoma Non-Small Cell | KEAP1 | R135L |
| NCI-H2126 | Carcinoma Non-Small Cell | KEAP1 | R272C |
| NCI-H1944 | Carcinoma Non-Small Cell | KEAP1 | R272L |
| NCI-H1623 | Carcinoma Non-Small Cell | KEAP1 | R320L |
| NCI-H2170 | Carcinoma Squamous Cell | KEAP1 | R336* |
| NCI-H1435 | Carcinoma Non-Small Cell | KEAP1 | R413L |
| NCI-H322T | Unknown | KEAP1 | R460S |
| H322T | Carcinoma Non-Small Cell | KEAP1 | R460S |
| NCI-H661 | Carcinoma Large Cell | KEAP1 | V168I |
| NCI-H2030 | Carcinoma Non-Small Cell | KEAP1 | V568F |
| NCI-H2023 | Carcinoma Non-Small Cell | KEAP1 | W252C |
| H1573 | Adenocarcinoma | KEAP1 | A143P |
| NCI-H2172 | Carcinoma Non-Small Cell | KEAP1 | G430C |
| H1792 | Adenocarcinoma | KEAP1 | G462W |
| NCI-H2122 | Carcinoma Non-Small Cell | KEAP1 | R202G |
| HCC2270 | Adenocarcinoma | NRF2 | G31E |
| NCI-H2228 | Carcinoma Non-Small Cell | NRF2 | G31A |
| NCI-H1568 | Carcinoma Non-Small Cell | NRF2 | DEE77-79 |
| EBC-1 | Carcinoma Non-Small Cell | NRF2 | D77V |

### C. Splice Variant Analysis

Analysis of splice variants was performed using the SGSeq software package available from the Bioconductor project website (Gentleman et al. Genome Biol. 5: R80, 2004). Exons and splice junctions were predicted from BAM files for 7,384 TCGA samples at 54 genomic loci of known oncogenes using parameters alpha=2, psi=0, beta=0.2, gamma=0.2. Predicted features were merged across samples, and exons were processed into disjoint exon bins. Splice junctions and exon bins were assembled into a genome-wide splice graph. Splice events, which consist of two or more alternative splice variants, were identified from the graph. Splice variants were quantified in terms of FPKM and relative usage W. Briefly, local estimates of relative usage at the start and end of the variant were obtained as the fraction of fragments that are compatible with the variant. Estimates at the event start and end were combined using a weighted mean, with weights proportional to the total number of fragments spanning the boundary. Relative usage estimates with denominator less than 20 were set to NA. To obtain a local estimate of absolute expression at the variant start and end, compatible counts n were converted to FPKMs as n I (N×L)×10⁹ where N is the total number of aligned fragments and L is the effective length (the number of allowed positions for a compatible fragment). Splice variants detected in TCGA samples were also quantified in 2,958 genotype-tissue expression project (GTEx) samples from normal human tissues (Consortium. Science. 348:648-660, 2015).

### D. Identification of Cancer-specific Splice Variants

Only internal splice variants (not involving alternative transcript starts or ends) were considered and the start and end of each splice variant were required to either overlap or extend exons that belong to annotated ref Gene transcripts downloaded from the UCSC Genome Browser website (Pruitt et al. Nucleic Acids Res. 33: D501-504, 2005; Rosenbloom et al. Nucleic Acids Res. 43: D670-681, 2015). Retained introns were excluded. 19 TCGA indications that included at least 100 cancer samples (6,359 cancer samples in total) were considered and splice variants with (i) FPKM>2 and relative usage ψ>0.2 in at least one cancer sample and (ii) FPKM<1 in> 99.9% of GTEx samples, and (iii) FPKM ˜ 0 in >97.5% of GTE x samples were selected. FPKM-based criteria were required to be satisfied at both the start and end of the splice variant. Variants satisfying the FPKM-based criteria for which Y could not be estimated were included after manual inspection.

### E. Analysis of Targeted Paired-end Exome-seq Data

All samples within FoundationCORE were processed and sequenced similarly as previously described (Frampton et al. Nat. Biotechnol. 31, 1023-1031, 2014). NRF2 exon 2 and exon 2+3 deletions were screened across a FoundationCORE dataset (n=58,707) using two distinct approaches.

First, rearrangement calls based on discordant read pairs and/or split reads were examined for direct evidence for loss of NRF2 exon 2 or exon 2+3. Although this approach provides direct evidence of the deletions of interest, deletions can only be discovered with this approach if the breakpoints are within a baited region because intronic regions of NRF2 are not captured. Thus, this approach identifies a limited subset of NRF2 exon 2 or exon 2+3 deletions in which the breakpoints occur near intron-exon boundaries or within exons.

The second approach utilizes copy number log ratio data from individual bait regions. Copy number log ratio values were determined with an in-house algorithm, educated to the specific tumor cellularity of each sample. A z-score was calculated comparing the log ratio for each exon in NRF2 to control polymorphism capture regions immediately adjacent to NRF2 (n=15; evenly spaced every ˜1MB from ˜3MB upstream and ˜12MB downstream of NRF2). Exon 2 deletions with and without concurrent exon 3 deletion were specifically examined. These are herein referred to as exons of interest (EOI). EOI deletions were called if (1) a z-score was <−2 for EOI and not for non-EOls in NRF2 and (2) a log-ratio drop of 0.2 from non-EOls in NRF2 was calculated. Mutual exclusivity between NRF2 exon 2 or exon 2+3 deletions and short variants in NRF2 or KEAP1 was examined specifically within lung squamous cell carcinoma (n=1,218).

### F. Cell Culture

KMS-27 (RPMI-1640), JHH-6 (Williams Media E), HuCCT1 (RPMI-1640), and HUH-1 (DMEM) cells were from JCRB, and 293 (EMEM) cells were from ATCC. Cells were cultured in the indicated media in the presence of 2 mM glutamine and 10% FBS.

### G. Western Blotting

Cell lysates were prepared with RIPA Buffer (Sigma) supplemented with complete EDTA-free protease inhibitor (Roche) and phoSTOP (Roche), Phosphatase Inhibitor Cocktail 2 (Sigma) and Phosphatase Inhibitor Cocktail 3 (Sigma) phosphatase inhibitors. Lysates were run on Novex Tris-Glycine 4-12% gradient gels (ThermoFisher) and transferred onto iBlot nitrocellulose (Invitrogen). Blots were pre-incubated in 5% skim milk powder (Merck) in TBST (10 mM Tris pH8, 150 mM NaCI, 0.1% TWEEN-20), followed by 5% bovine serum albumin (Sigma) in TBST containing antibodies. Secondary antibodies used were ECL Anti-Rabbit HRP and ECL Anti-Mouse HRP (both from GE Heathcare). Blots were developed with a Chemiluminescence Substrate Kit (Protein Simple) and visualized with a FluorChem HD2 imager (Protein Simple). Antibodies used in this study are against KEAP1 (Cell Signaling G1010), NRF2 (Abcam ab62352), HSP90 (Cell Signaling 4877), HDAC2 (Cell Signaling 5113), B-actin (Sigma A2228), HA (Roche 11815016001), and FLAG (Sigma F2426). Lamda phosphatase was from NEB (P0753L), and phosphatase inhibitors were omitted from the lysis buffer in these experiments.

### H. Cell Viability and DNA Fragmentation Analysis

siRNAs were reverse transfected into cells with Dharmafect 2 reagent (ThermoFisher) and OptiMEM (Gibco). Four days post transfection, cells were measured for viability using CellTiter-Glo reagent (Promega) and luminescence was detected on an EnVision Multi-label Reader (Perkin Elmer). siRNAs were reverse transfected into cells with Dharmafect 2 reagent (ThermoFisher) and OptiMEM (Gibco). Four days post-tranfection, cells were measured for apoptosis using propidium iodide (PI) (LifeTechnologies) staining and flow cytometry following a published protocol (Riccardi and Nicoletti Nat. Protoc. 1:1458-1461, 2006). Staurosporin, 1 μM, (Enzo) was added to positive control cells 24 hours pre-staining. siRNAs targeting NRF2 exon 2 had the sequences: 5′-TGGAGTAAGTCGAGAAGTA-3′ (SEQ ID NO: 29) and 5′-ACAACTAGATGAAGAGACA-3′ (SEQ ID NO: 30). siRNAs targeting NRF2 exon 5 had the sequences: 5′-TGACAGAAGTTGACAATTA-3′ (SEQ ID NO: 31) and 5′-GTAAGAAGCCAGATGTTAA-3′ (SEQ ID NO: 32), and were used along with non-target siRNA as control siRNA. Stained cells were analyzed with a Becton Dickinson FACS Caliber instrument. siRNAs targeting KEAP1 were from Dhamacon (L012453-00).

DNA fragmentation was quantified by propidium iodide (PI) staining and measured by flow cytometry according to Riccardi et al. (Nature Protocols, 1:1458-1461 (2006)).

### I. Taqman Analysis

Total cellular RNA was extracted with an RNeasy Kit (Qiagen). RNA was converted to cDNA using a High Capacity cDNA Reverse Transcription Kit (Applied Biosystems), and cDNAs were amplified with Taqman Gene Expression primer-probe sets (ThermoFisher) using Taqman Gene Expression Master Mix reagents (Applied Biosystems). Taqman amplification/detection was performed on a QuantStudio7 Flex Real-Time PCR System. Primer-probe sets used were Hs00232352_ml and Hs00975961_gl to detect NRF2 exons 2 and 5, respectively (ThermoFisher). NRF2 target gene Taqman primer-probe sets used were: SLC7A11 (Hs00921938_ml), SGRN (Hs00921938_ml), NROB1 (Hs03043658_ml), GCLC (HsOOI55249_ml), and GPX2 (Hs01591589_ml), all from ThermoFisher.

### J. 293 Transfections

Plasmid DNAs were transfected into cells using Lipofectamine 2000 (ThermoFisher) and OptiMEM (Gibco) as recommended by manufacturer protocol. Lysates were prepared 2-3 days post transfection. Expression plasmids used were pRK5.NRF2, pRK5.NRF2.delta.e2, pRK5.NRF2.delta.e2,3, pRK5.NRF2.FLAG and pRK5.KEAP1.HA.

### K. Tumor Xenograft Models

Eleven to twelve week-old female C.B-17 SCID.beige mice (Charles River Laboratories) were subcutaneously inoculated in the right lateral flank with 10×10⁶ A549 shRNA cells in 100 μl HBSS/MATRIGEL® (BD Biosciences) or with 10×10⁶ H441 shRNA cells in 100 μl HBSS per mouse. When tumor volume reached approximately 150-250 mm³, mice were randomized to receive drinking water containing 1 mg/ml doxycycline (in 5% sucrose) or no doxycycline (5% sucrose alone) ad libitum. The doxycycline was replaced 3 times a week and the sucrose replaced once a week. Tumor volumes were determined using digital calipers (Fred V. Fowler Company, Inc.) using the formula (L×W×W)/2 and plotted as mean tumor volume (mm³)+/−SEM. Tumor growth inhibition (% TGI) was calculated as the percentage of the area under the fitted curve (AUC) for the respective dose group per day in relation to the vehicle, such that % TGI=100×1−(AUC treatment/day)/(AUC vehicle/day). In a separate study, mice with 150-250 mm³ tumors were dosed with 1 mg/ml doxycycline for 5 days before the tumors were excised and analyzed by Western blotting for NRF2 levels.

L. A549 Xenografts Treated with ErbB3 Antibodies

Female nude mice (n=10) bearing subcutaneous A549 tumors (75-144 mm3) on Day 1 were treated with vehicle or 50 mg/kg YW57.88.5 (100 mg/kg loading dose) administered intravenously once each week for four weeks (qwk×4). Tumors were measured twice each week, and each animal was euthanized for endpoint at the earlier of its tumor reaching a volume of 1000 mm³ or on the final day of the treatment regimen.

### Example 2

### Identification of NSCLC Cell Lines with Mutations in KEAP1 and NRF2

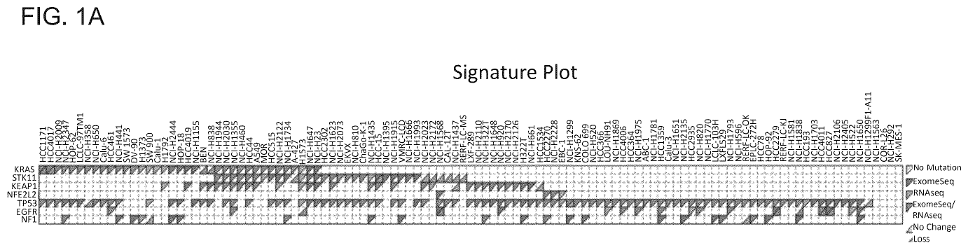

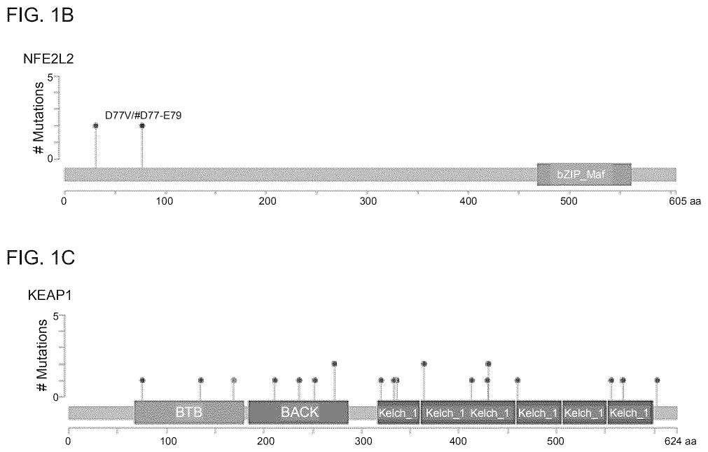

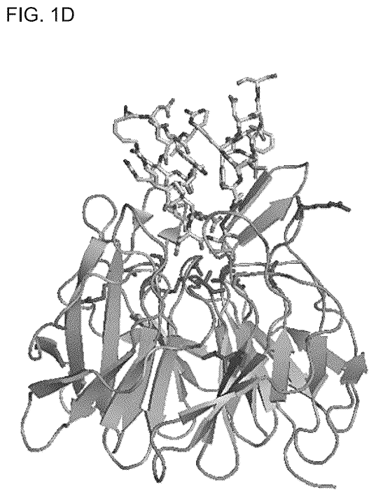

To identify mutations, copy number, and loss of heterozygosity (LOH) of KEAP1 and NRF2 in NSCLC, a panel of 113 NSCLC cell lines profiled by RNA-seq, exome-seq, or SNP arrays was documented (FIG. 1A). KEAP1 mutations were found in 29/113 cell lines (26%), and NRF2 mutations were detected in 4/113 cell lines (4%). Except for the NCI-H661 cell line, all KEAP1 mutated cell lines showed homozygous expression of the mutated allele, which was generally associated with copy neutral LOH. In contrast, the NRF2 mutations were heterozygous and not associated with LOH. A further two cell lines (HCC1534 and NCI-HI437) showed no detectable KEAP1 mRNA through bi-allelic loss of KEAP1 DNA. The NRF2 mutations were in the previously identified hotspots in the KEAP1 interface regions (FIG. 1B) (Shibata et al. Proc. Natl. Acad. Sci. U.S.A. 105:13568-13573, 2008), and comprised point mutations and an in-frame 3-amino-acid deletion. The mutations in KEAP1 were spread throughout the primary sequence (FIG. 1C), with few obvious hotspots. However, when mapped onto the KEAP1/NRF2 peptide crystal structure (Fukutomi et al. Mol. Cell. Biol. 34:832-846, 2014), the mutations cluster in the loops extending from the KEAP1 core beta propeller close to the interaction site with NRF2 (FIG. 1D).

### Example 3

### Identification of a Mutant KEAP1 Gene Signature

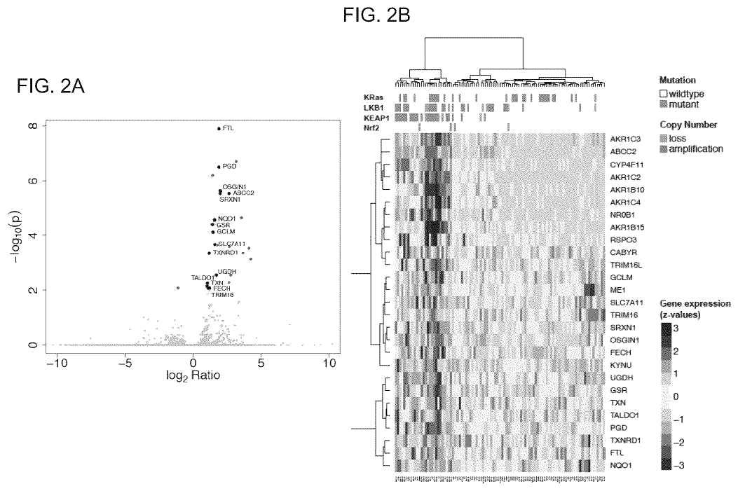

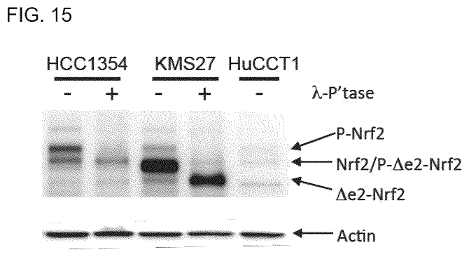

To determine the transcriptional consequences of KEAP1 mutations in NSCLC cell lines, genes that were significantly differentially expressed (p<0.01, absolute mean fold-change>2) in the KEAP1 mutated cell lines compared to the wild-type KEAP1 cell lines were identified. Overall, 27 genes were significantly up-regulated in the KEAP1 mutant cell lines (FIGS. 2A-2B), 15 of which have previously been identified as NRF2 target genes from ChIP-seq or RNA-seq studies (Chorley et al. Nucleic Acids Res. 4:7416-7429, 2012; Hirotsu et al. Nucleic Acids Res. 40:10228-10239, 2012; Malhotra et al. Nucleic Acids Res. 38:5718-5734, 2010). Only one gene, HSPB1, was identified as significantly down-regulated using these cut-offs.

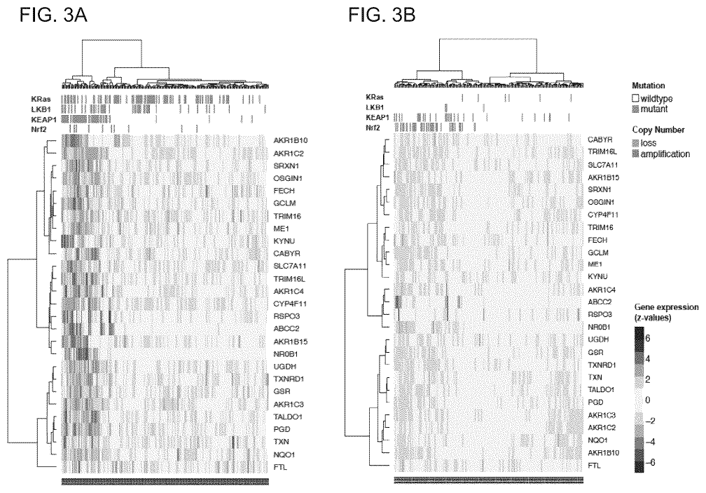

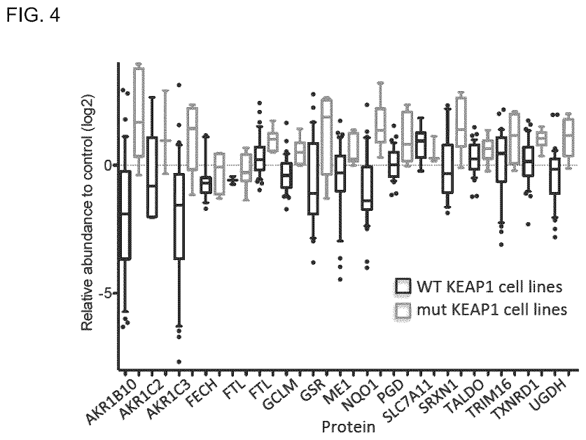

Unsupervised clustering of 230 TCGA lung adenocarcinomas based on the expression of these 27 genes resulted in the division of two major groups (FIG. 3A). One group was mainly characterized by high expression of the 27 signature genes, and contained 43 tumors, out of which 32 (74%) were KEAP1 mutant. The other group, characterized by low expression, contained 187 tumors, out of which 179 were KEAP1 wild-type. Strikingly, using the same gene set to cluster lung squamous cell carcinomas, NRF2 as well as KEAP1 mutant tumors were distinguished from the NRF2/KEAP1 wild-type tumors (FIG. 3B), suggesting that NRF2 mediates most of the transcriptional consequences of KEAP1 loss/mutation. Interestingly, there were several squamous NSCLC tumors that showed high expression of the KEAP1 mutant genes without any known mutations in either KEAP1 or NRF2. Of the 27 genes up-regulated in the KEAP1 mutant cell lines, proteomic data was available for 17 in a smaller sub-set of cell lines (37 wild-type KEAP1, 6 mutant KEAP1). Consistent with the increased levels of mRNA of these genes in the mutant KEAP1 cell lines, the protein targets of all but one of these 17 genes (SLC7A11, which had low peptide coverage) also showed increased expression in the mutant KEAP1 cell lines relative to the wild-type cell lines (FIG. 4).

### Example 4

### Identification of Aberrant Splicing of NRF2 in Tumor Samples

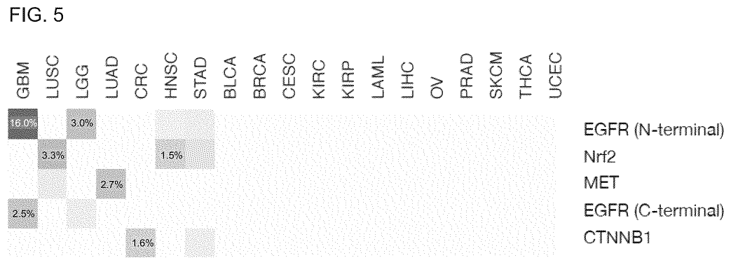

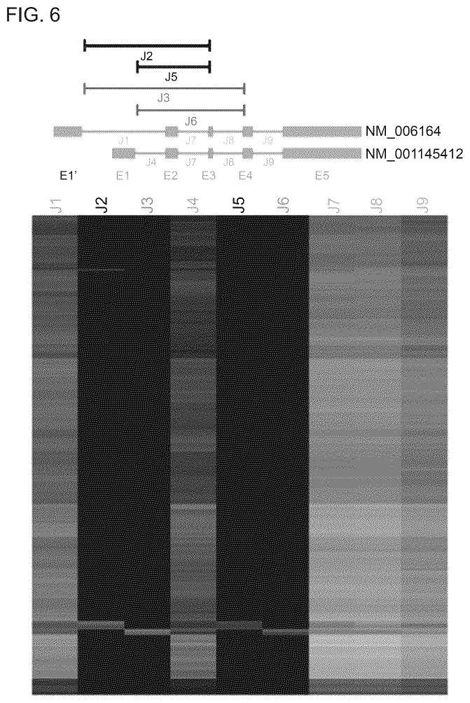

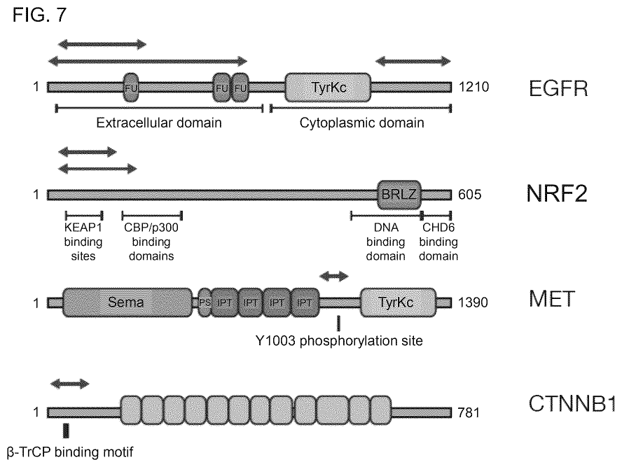

For the majority of tumors with high expression of the 27 candidate NRF2 target genes, elevated gene expression could be explained by mutations in KEAP1 or NRF2. However, there were some tumors that showed high expression of candidate NRF2 target genes in the absence of characterized mutations in either KEAP1 or NRF2. Cancer-associated transcript alterations are increasingly recognized as possible driver events. Therefore, it was hypothesized that NRF2 pathway activation in these tumors might be driven by splice alterations not recognized by whole-exome sequencing. 54 known oncogenes were analyzed to identify splice variants that are recurrently observed in cancer samples from the TCGA but are rarely detected in normal samples from the GTEx (see Example 1). 19 cancer types were selected, each including at least 100 cancer samples (6,359 samples in total). In the 54 considered oncogenes, nine recurrent candidate cancer-specific splice variants were identified (≥ 2 samples and >1% of samples for a given cancer type). Using the same detection criteria as in the cancer samples, none of these variants could be detected in normal controls (2,958 samples in total). Grouping together related variants with shared splice sites yielded five independent alterations in four oncogenes (FIG. 5). These alterations included several well-documented oncogenic splice variants, including EGFRvIII in brain cancers, MET exon 14 skipping in lung adenocarcinoma and CTNNB1 exon 3 deletions in colorectal cancers (Cho et al. Cancer Res. 71 (24): 7587-7596, 2011; Kong-Beltran et al. Cancer Res. 66 (1): 283-289, 2006; Iwao et al. Cancer Res. 58 (5): 1021-1026, 1998). Interestingly, previously uncharacterized splice variants in NRF2 were observed and occurred frequently in patients with squamous NSCLC (3.3%; 16/481) and at lower prevalence in patients with HNSC (1.5%; 6/403) (FIG. 5A). A more detailed analysis of NRF2 splice variants in lung squamous carcinoma revealed two splice variants co-occurring in the same patients, corresponding to a skip of NRF2 exon 2 in mRNAs transcribed from either one of two alternative promoters (2.1%; 10/481) (FIG. 6). Two additional splice variants co-occurred in a distinct set of patients (1.2%; 6/481), corresponding to a skip of both NRF2 exons 2 and 3 (exon 2+3) in mRNAs with either one of the two alternative transcript starts (FIG. 6). All patients expressing NRF2 splice variants lacking exon 2 or exon 2+3 also showed expression of normal NRF2 transcripts as evidenced by split reads supporting inclusion of exon 2. Both exons 2 and 3 are part of the NRF2 coding sequence, and skip of exon 2 or exon 2+3 are predicted to result in protein isoforms with either an N-terminal truncation or an in-frame deletion (FIG. 7). The high recurrence of NRF2 transcripts lacking exon 2 and preservation of coding potential suggest that these splice variants may present gain-of-function events conferring a selective advantage. This is supported by the finding that exon 2 encodes the Neh2 domain, which allows for interaction with KEAP1 (Itoh et al. Genes Dev. 13 (1): 76-86, 1999), which is mutated in 15% of squamous lung cancers.

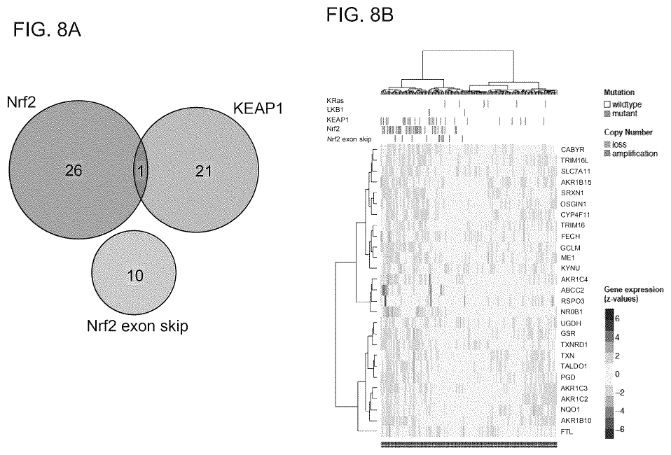

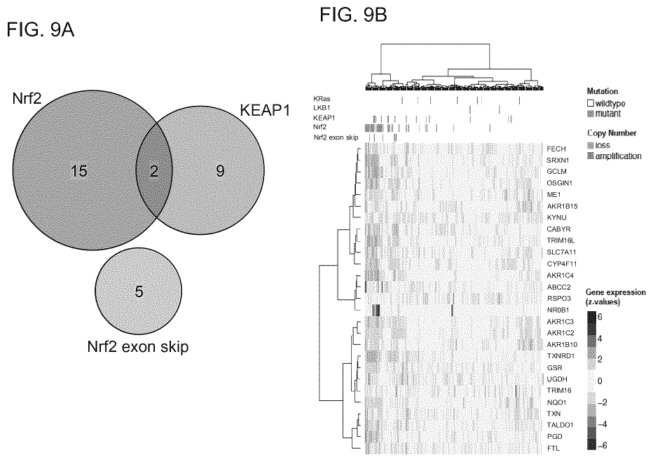

To assess whether the observed NRF2 splice variants can account for NRF2 pathway activation in patients without mutations in KEAP1 or NRF2, co-occurrence of NRF2 splice variants and NRF2 pathway mutations were observed. In the TCGA collection, 178 of the squamous lung tumors were profiled by exome-seq. In this subset, 10 tumors (6%) displaying exon 2 or exon 2+3 deletion were mutually exclusive with 48 tumors (27%) showing mutations in either NRF2 or KEAP1 (FIG. 8A). Moreover, all exon-2 deleted tumors showed high expression of the 27 candidate NRF2 target genes (FIG. 8B). Similar observations were made for head and neck cancer, where NRF2 exon deletion in 5 tumors (2%) were mutually exclusive with mutations in NRF2 or KEAP1 in 26 tumors (9%) (FIGS. 9A-9B). These results suggest that deletion of exon 2 represents an alternative mechanism for activation of NRF2 in a subset of squamous NSCLC and head and neck tumors. Importantly, these results show that consideration of splice alterations, in addition to exome sequencing, increased the percentage of patients identified as having putative NRF2 pathway activation from 27% (48/178) to 33% (58/178) in lung squamous carcinoma and from 9% (26/275) to 11% (31/275) in head and neck squamous carcinoma.

### Example 5

### Validation of NRF2 Splicing Defects in Cell Lines

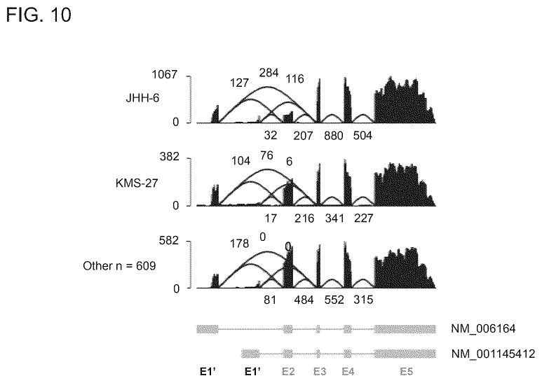

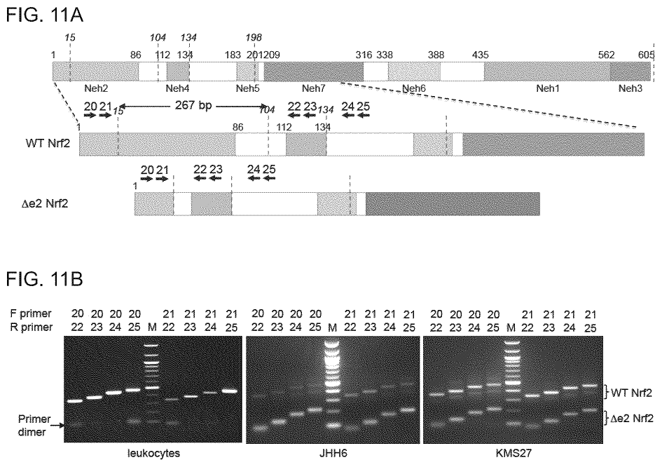

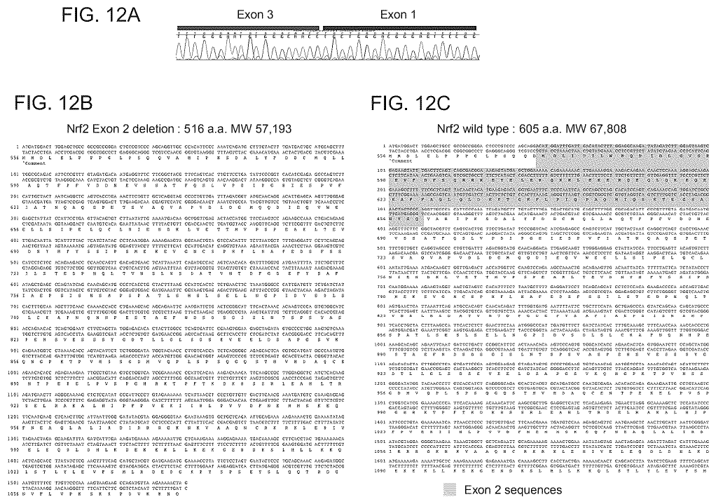

To identify cell line models for further study, read evidence for the identified splice variants in RNA-seq data was analyzed from a large panel of human cancer cell lines (described in Klijn et al. Nat. Biotechnol. 33 (3): 306-312, 2014). Out of 611 cell lines, one multiple myeloma cell line KMS-27 and one hepatocellular carcinoma cell line JHH-6 were identified, both showing evidence for heterozygous skip of NRF2 exon 2 by junction reads (FIG. 10). The NRF2 exon 2 skipping by RT-PCR in JHH-6 and KMS-27 mRNA was validated. Using a series of forward and reverse primers derived from exon 1 and exons 3/4 respectively (FIG. 11A), the exon 2 deletion (Ae2 NRF2) in mRNA isolated from JHH-6 and KMS-27 cells was confirmed (FIG. 11B). Sequencing of the PCR products confirmed the expected deletion of exon 2 (FIGS. 12A-12C). Based on RNA-seq data no point mutations were detected in the coding sequence of NRF2 or KEAP1 in JHH-6 or KMS-27 (Klijn et al. Nat. Biotechnol. 33 (3): 306-312, 2014).

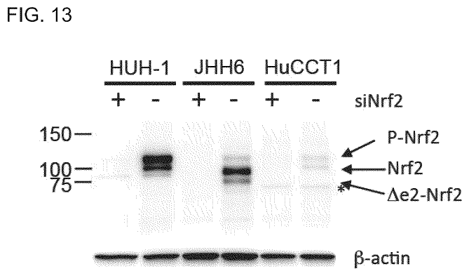

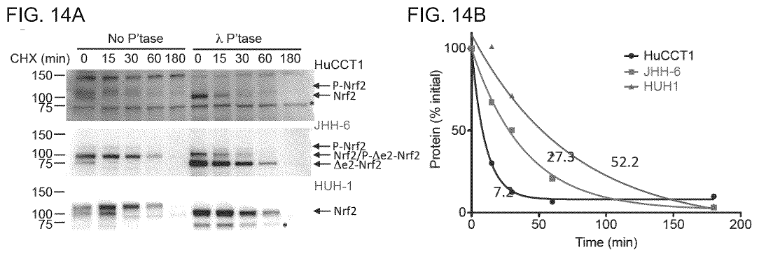

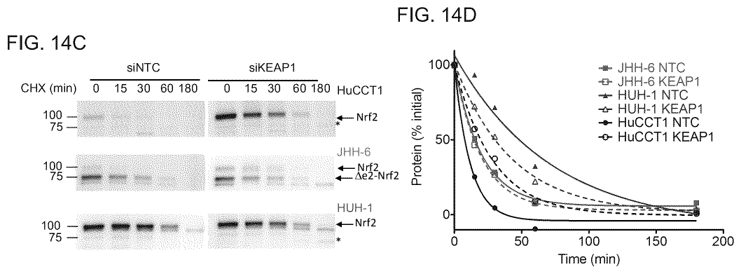

As NRF2/KEAP1 alterations are fairly common in hepatocellular carcinoma (10%) but infrequent in multiple myeloma (0%), JHH-6 cells were further tested. Specifically, expression of the exon 2-deleted form of NRF2 protein was tested. Western blotting of whole-cell lysates from JHH-6 cells, as well as the KEAP1 mutant HUH-1 line, and HuCCT1 cells as a representative wild-type KEAP1 liver cancer cell line was performed. The levels of NRF2 in JHH-6 cells were comparable to those seen in HUH-1 cells, which were much higher than in the wild-type KEAP1 HuCCT1 cells (FIG. 13). Moreover, a smaller molecular weight species, consistent with a deletion of exon 2, was detectable in JHH-6 and was reduced upon NFE2L2 siRNA transfection, confirming that it indeed represents a form of NRF2. While the altered NRF2 isoform was visible, it was surprising that it was not more abundant, given the lack of KEAP1 interaction motifs. It was hypothesized that a phosphorylated form of exon 2-deleted NRF2 might co-migrate with the unphosphorylated form of wild-type NRF2 in the 4-12% gels used. Indeed, dephosphorylation of JHH-6 lysates showed that the exon 2-deleted form of NRF2 was significantly more abundant than the wild-type form (FIG. 14A, middle panel). Similarly, KMS-27 cells expressed the exon 2-deleted form of NRF2, which was the major species apparent upon dephosphorylation (FIG. 15).

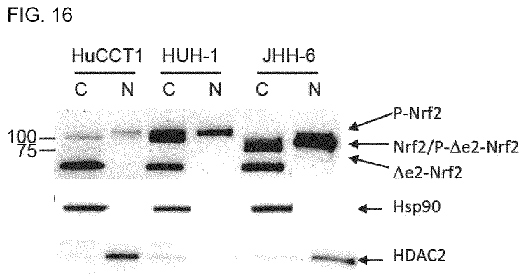

The stability of NRF2 in the three liver cancer cell lines was tested using cycloheximide to abolish total protein synthesis. Dephosphoryalted lysates were used to allow more accurate quantification of total NRF2. The experiments showed increased stability of Ae2 NRF2 in JHH-6 cells, comparable to NRF2 in HUH-1 cells, which were both more stable than NRF2 in HuCCT1 cells (FIGS. 14A-14B). The exon 2-deleted form of NRF2 in JHH-6 cells also showed prominent nuclear localization, also when compared to HUH-1 cells (FIG. 16).

To determine whether the deletion of exon 2 in JHH-6 cells made NRF2 refractory to regulation by KEAP1, the stability of NRF2 in response to KEAP1 knockdown was tested. Knockdown of KEAP1 in

HuCCT1 cells resulted in increased steady state levels of NRF2 due to increased stability (FIG. 14C). However, knockdown of KEAP1 in JHH-6 cells did not affect the levels or stability of exon 2-deleted NRF2. As expected, knockdown of KEAP1 did not increase the stability of wild-type NRF2 in the KEAP1 mutant HUH-1 cell line (FIG. 14D).

### Example 6

### Assessment of Exon 2 and/or Exon 2+3 Deletion on NRF2

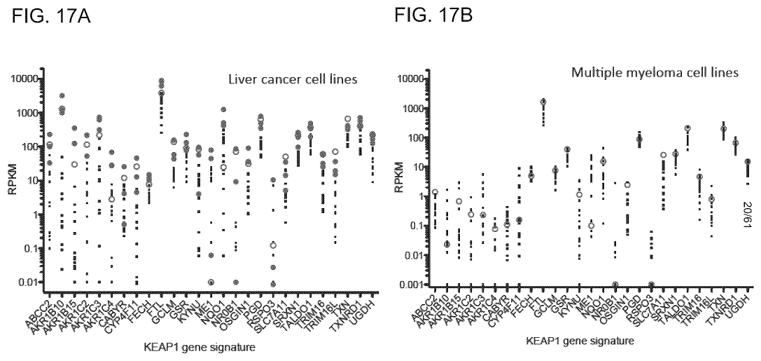

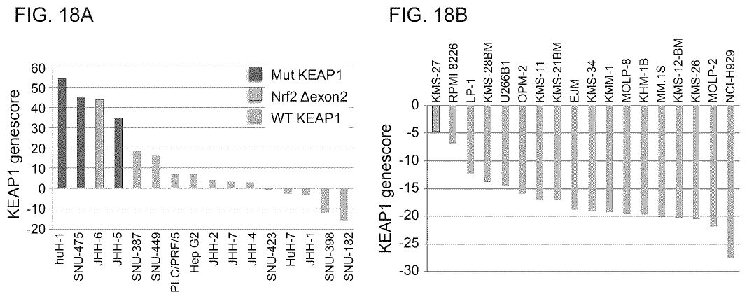

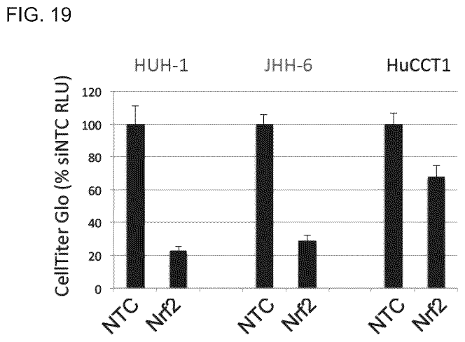

Utilizing the NRF2/KEAP1 gene signature described in Example 3, it was determined that, of 16 hepatocellular carcinoma cell lines, JHH-6 cells show among the highest expression of NRF2 target genes from RNA-seq data, similar to those seen in mutant KEAP1 expressing lines (FIG. 17A). Similarly, out of 18 multiple myeloma cell lines examined, KMS-27 cells show among the highest expression of these genes (FIG. 17B). Expression of these genes can be summarized by a “NRF2 target gene score” calculated as the mean of z-scores for individual target genes across the 611 cell lines examined. This results in a single score per cell line that reflects the extent of overexpression of signature genes in the given line. The NRF2 target score confirms that JHH-6 cells show a similar score as liver cancer cell lines expressing KEAP1 mutations (FIG. 18A) and KMS-27 cells show the highest score among multiple myeloma cell lines (FIG. 18B), despite multiple myeloma showing a low overall NRF2 target gene score (indicated by the negative values).

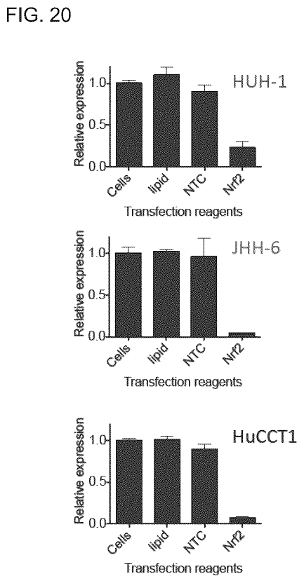

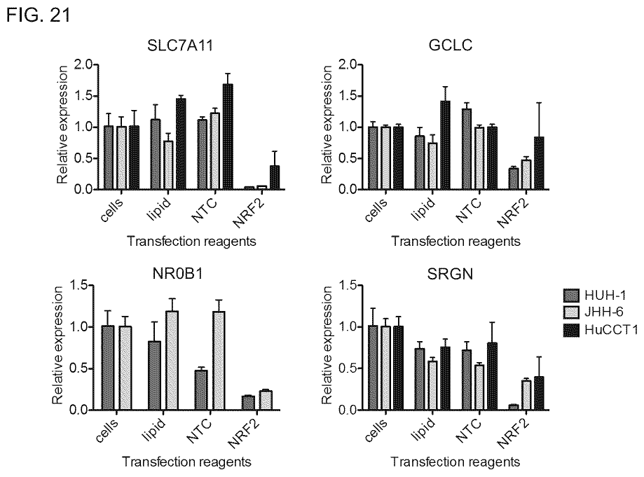

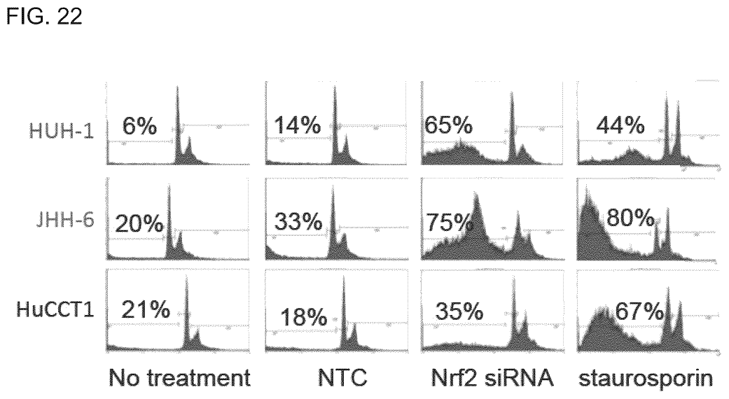

Next, the dependence of JHH-6 cells expressing exon 2 deleted NRF2 on the expression of NRF2 protein was compared to wild-type NRF2 expressing HuCCT1 cell. Knockdown of NRF2 in JHH-6 cells caused a marked decrease in cell viability, similar to that seen in the mutant KEAP1 hepatocellular carcinoma cell line HUH-1. In contrast, NRF2 knockdown had a more modest effect on the viability of HuCCT1 cells (FIG. 19). This was not due to defective NRF2 knockdown in HuCCT1 cells, as NRF2 knockdown was equally efficient in all three cell lines (FIG. 20). Knockdown of NRF2 also resulted in decreased expression of four well-characterized NRF2 target genes, although this was slightly reduced in the wild-type KEAP1HuCCT1 cell line (FIG. 21). Decreased viability was likely due, at least in part, to apoptosis as measured by an increase in fragmented DNA (FIG. 22).

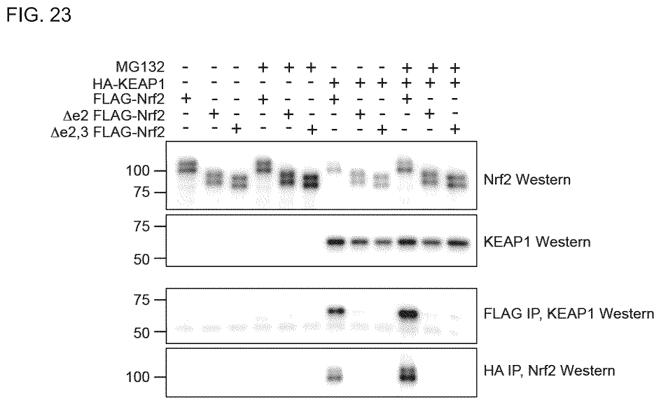

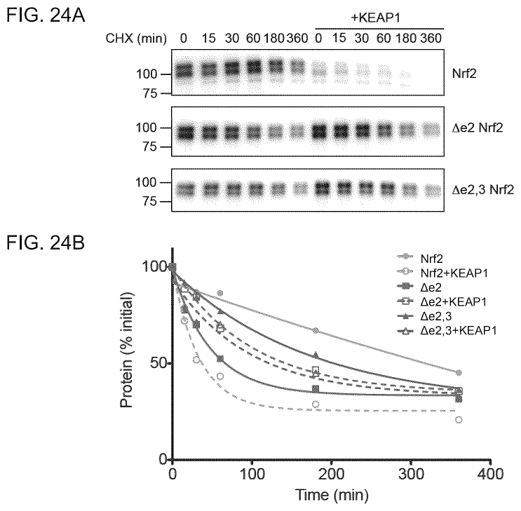

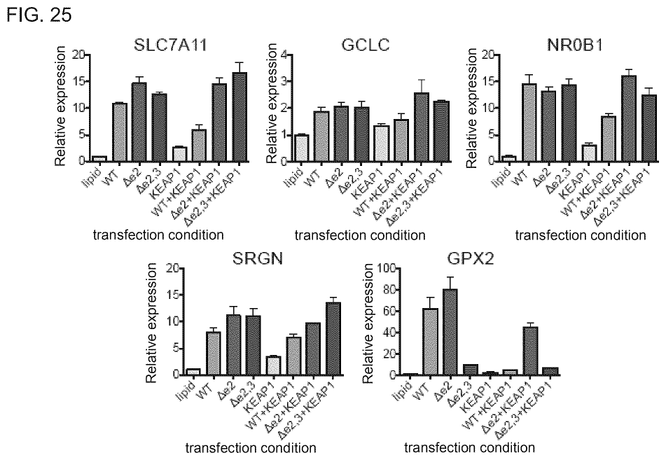

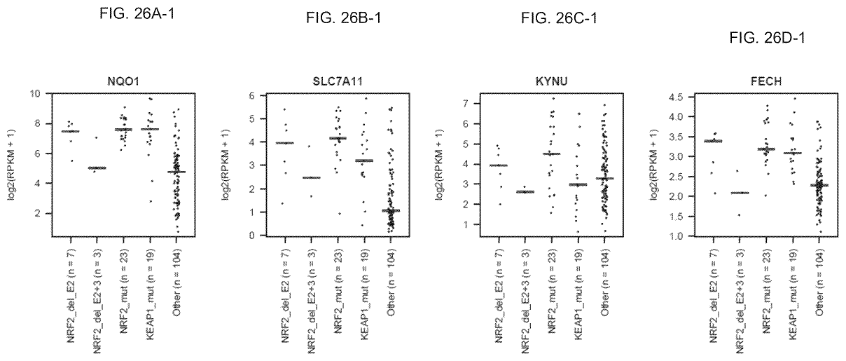

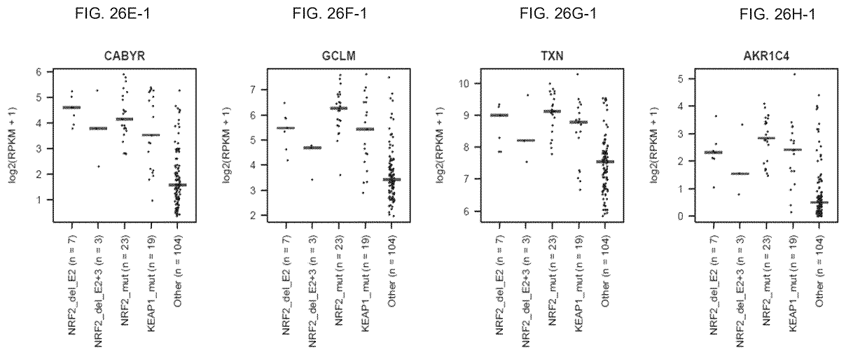

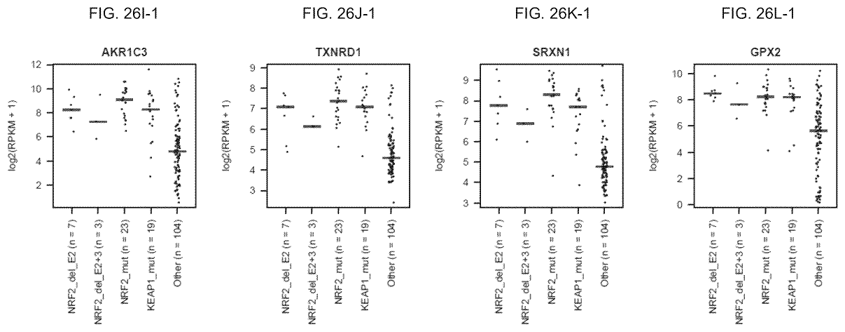

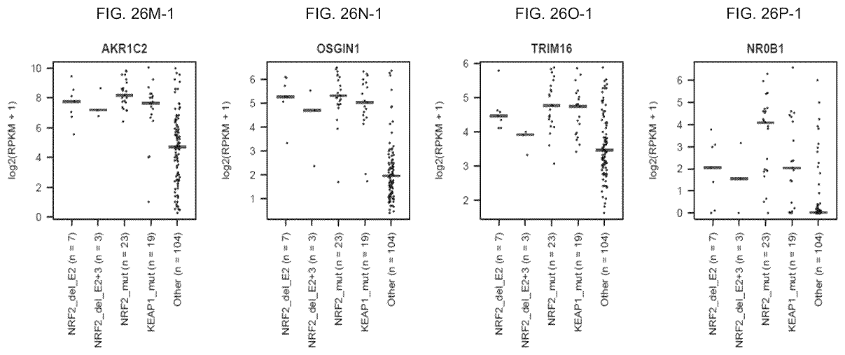

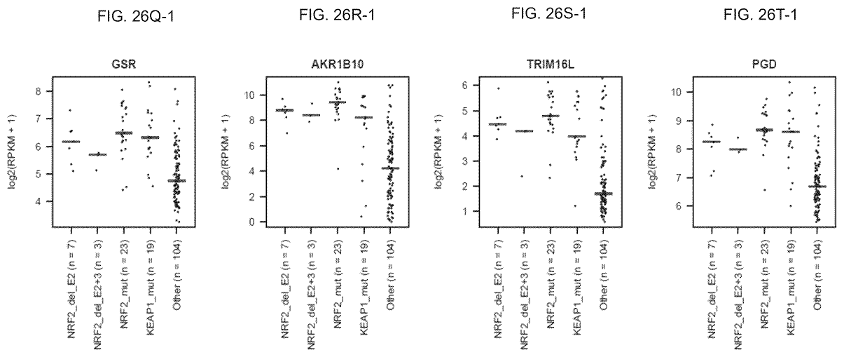

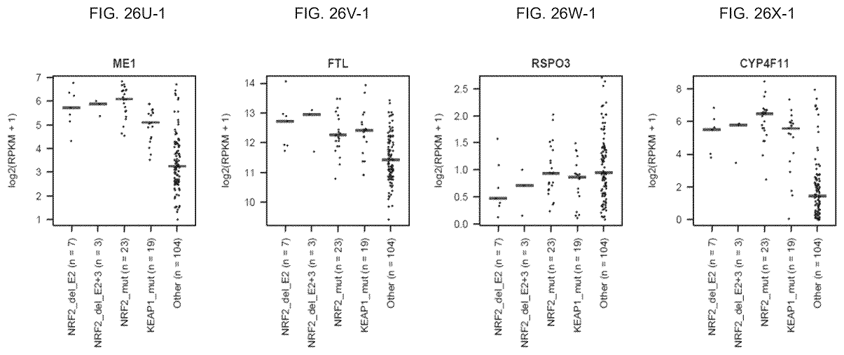

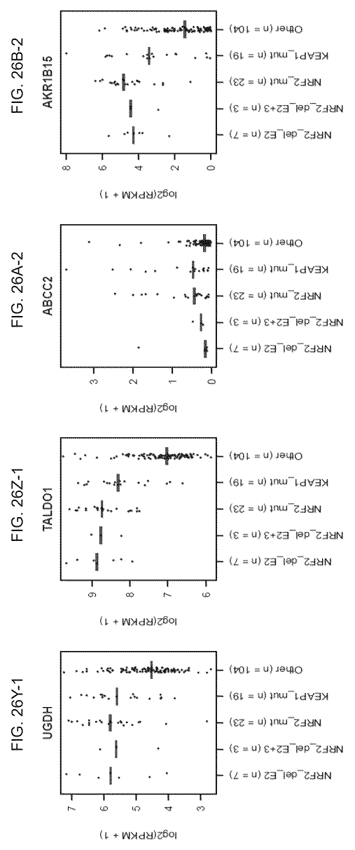

To address how loss of NRF2 exon 2 affects the ability of NRF2 to be regulated by KEAP1, transient expression in 293 cells was used. KEAP1 decreased the expression of full-length NRF2, but had lesser effects on the expression of NRF2 lacking exon 2 or exons 2+3 (FIG. 23, upper panels). The inhibitory effect of KEAP1 on full-length NRF2 expression was mostly abolished by proteasome inhibitor MG132, as expected. Full-length NRF2 and KEAP1 interacted with each other, whereas deletion of exon 2 or exon 2+3 completely abolished the ability of KEAP1 to bind NRF2 (FIG. 23, lower panels). As a result, truncated NRF2 remained stable following KEAP1 expression, in contrast to wild-type NRF2 (FIGS. 24A-24B), although the truncated forms of NRF2 appeared to have slightly decreased intrinsic stability. However, altered NRF2 isoforms were transcriptionally active, as judged by their ability to increase NRF2 target gene expression (FIG. 25). Most genes were similarly increased by exon 2-or exon 2+3-deleted NRF2 compared to full-length NRF2 and were resistant to the effects of KEAP1 overexpression. Interestingly, exon 2+3-deleted NRF2 was defective for increasing GPX2 expression, suggesting that there might be subtle differences in the transcriptional activation of this form of NRF2. Consistent with this observation, 22 of the 27 target genes described in Example 3, in addition to GPX2, showed lower median expression in exon 2+3-deleted squamous lung tumors compared to exon 2-deleted tumors (FIG. 26).

### Example 7

### Mechanistic Analysis of NRF2 Exon 2 Splice Alteration

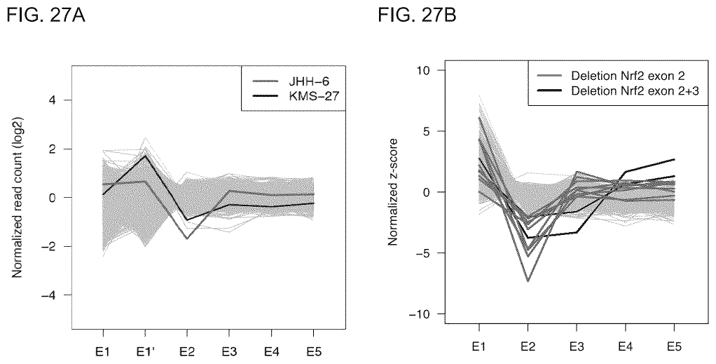

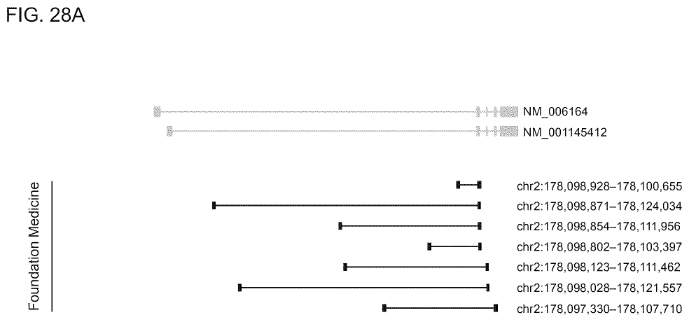

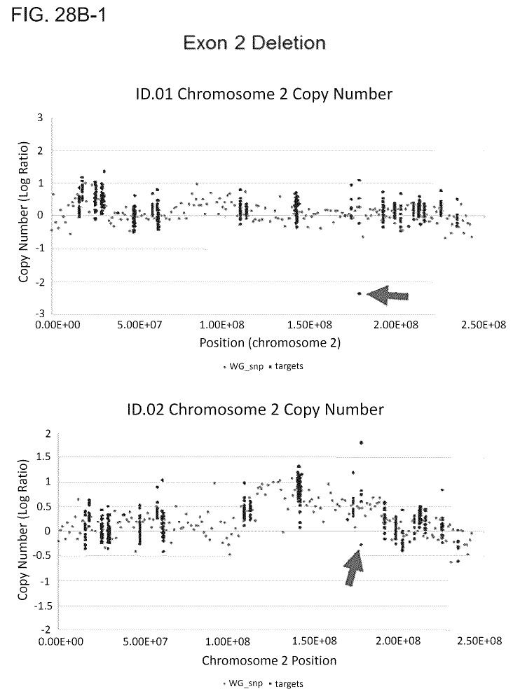

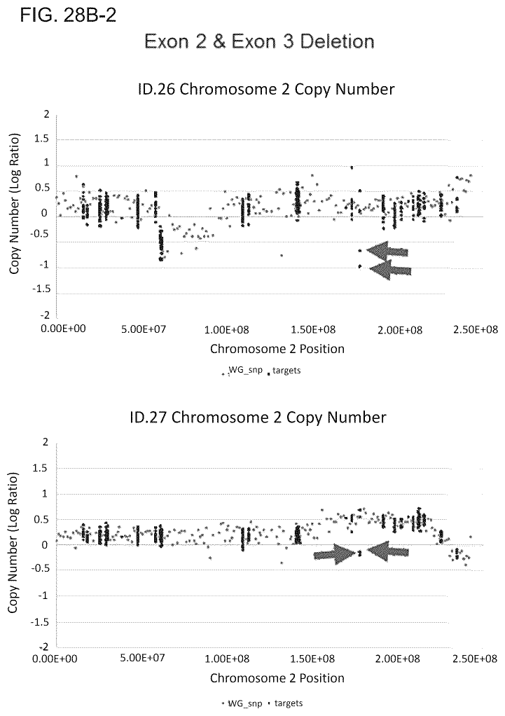

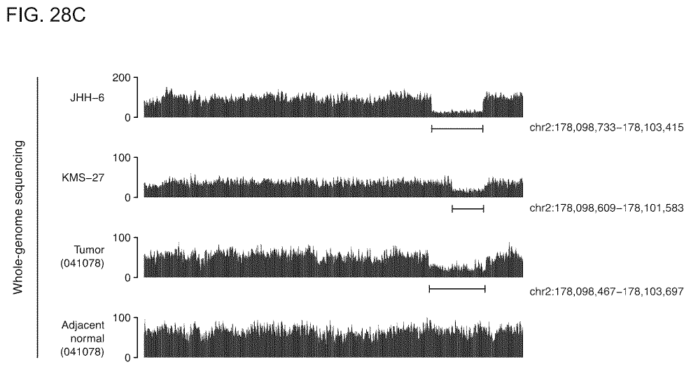

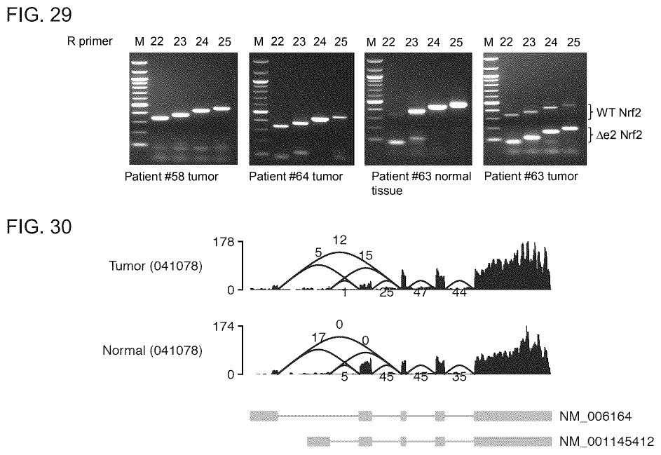

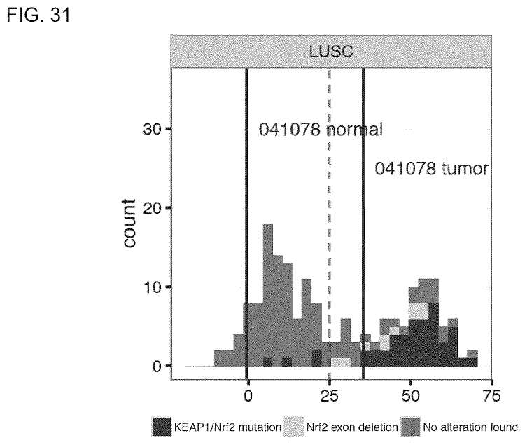

Analysis of exome-seq data for KMS-27 and JHH-6 shows a decrease in reads mapping to exon 2, suggesting that the observed transcript variants could be the result of genomic alterations (FIG. 27A). Whole-genome sequencing (WGS) of JHH-6 and KMS-27 showed that these cell lines harbor microdeletions surrounding NRF2 exon 2, spanning 4,685 and 2,981 nucleotides, respectively (FIG. 27B). To investigate the causal mechanism in patients, targeted paired-end exome-seq data from a large cohort (n=1,218) of clinical squamous NSCLC tumors with high read coverage (>300×) were analyzed. In this data set, eleven tumors showed a decrease in copy number for exon 2 or exons 2+3 compared to nearby control regions (Materials and Methods; FIG. 27B). The focal nature of the deletions can be appreciated by investigating log-ratios from defined genomic regions targeted for sequencing (FIGS. 28B-1 and 28B-2). Seven tumors with discordant read pairs were consistent with structural variants encompassing several kilobases of DNA and affecting exon 2 or exon 2+3 (FIG. 28A). In total, sixteen patients showed evidence for genomic alterations affecting NRF2 exon 2 or exon 2+3, and the identified events were mutually exclusive with point mutations or indels in NRF2 and KEAP1, which are known to activate this pathway. An additional cohort of 45 squamous NSCLC tumors were analyzed, for which both RNA and DNA were available. RT-PCR analysis identified a single patient with loss of exon 2, which was strongly enriched in the tumor compared to adjacent normal tissue (FIG. 29). RNA-seq analysis confirmed that the transcript variant was expressed in the identified tumor, but was absent in adjacent normal tissue (FIG. 30). Expression of NRF2 target genes was also elevated to a similar extent as in TCGA tumors with known mutations in this pathway, whereas the adjacent normal tissue showed low expression of these genes (FIG. 31). Finally, whole-genome sequencing confirmed that the transcript variant was the result of a somatic genomic microdeletion of 5,233 nucleotides surrounding exon 2 (FIG. 28C). These data suggest that genomic microdeletions are a clinically relevant mechanism for NRF2 pathway activation.

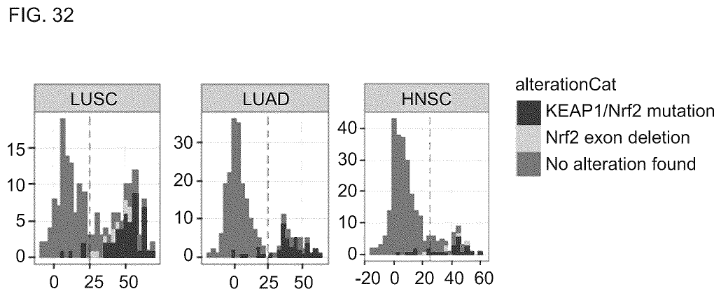

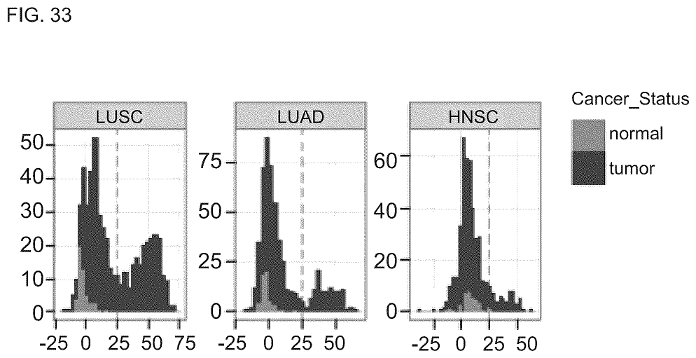

These data suggest that the set of genes regulated by NRF2 is conserved across different tissues and conditions. This has practical value in the use of a single gene signature to identify tumors with NRF2 activation in both NSCLC and HNSC (FIG. 32). Interestingly, this NRF2/KEAP1 signature is only activated in tumors. Matched normal samples for lung and head and neck tumors showed only low NRF2 target gene activity (FIG. 33). This suggests that inhibition of the NRF2 pathway might have selective benefit in tumors showing pathway deregulation compared to normal tissues.

Intragenic genomic deletions that result in activation of proto-oncogenes have previously been reported for a number of genes, including EGFR and CTNNB1. Such variants are not routinely assayed, due in part to limitations of current genomic technologies. In particular, small aberrations affecting individual exons and involving small copy number changes are difficult to detect by exome-seq alone. Thus, intragenic deletions have remained relatively unexplored and new variants are still being discovered. Recent studies of small cell lung cancer and adult T cell leukemia/lymphoma identified recurrent microdeletions in TP73, IKZF2, and CARD11 using whole-genome sequencing (George et al. Nature 524, 47-53:2015; Kataoka et al. Nat. Genet. 47:1304-1315, 2015). In the present study, publicly available RNA-seq data generated as part of the TCGA project was used to identify recurrent transcript alterations in known oncogenes. Due to differences between patient cohorts, it is difficult to assess the general prevalence of NRF2 exon deletions. For example, when analyzing TCGA lung squamous cancers with available RNA-seq data (n=481), we identified 3% (16/481) of patients having a deletion of NRF2 exon 2 or exon 2+3. When analyzing the subset of patients with available exome-seq data (n=178), for which somatic mutation calling can be performed, the proportion of patients with NRF2 exon deletions was 6% (10/178). Accounting for NRF2 exon deletions increased the percentage of patients with putative NRF2 pathway activation from 27% (48/178) to 33% (58/178) in lung squamous carcinoma and from 9% (26/275) to 11% (31/275) in head and neck squamous carcinoma, compared to assessing mutations in NRF2 or KEAP1 by exome-seq alone (FIGS. 8A and 9A). Analysis of real-world clinical samples from patients that underwent genomic profiling suggested a prevalence of NRF2 exon deletions in 1-2% of lung squamous cell carcinoma. However, the latter analysis lacks sensitivity since optimized criteria for determining single-exon deletions in samples with variable tumor content have yet to be established and only unambiguous deletions were considered. Nevertheless, the results presented herein are consistent with the concept that modulation of this pathway is frequently altered in specific tumor indications, such as squamous NSCLC and head and neck carcinomas. Additional screening of known cancer genes through sequencing of complete gene loci, including introns, or by combining data from exome and RNA sequencing experiments may also be performed.

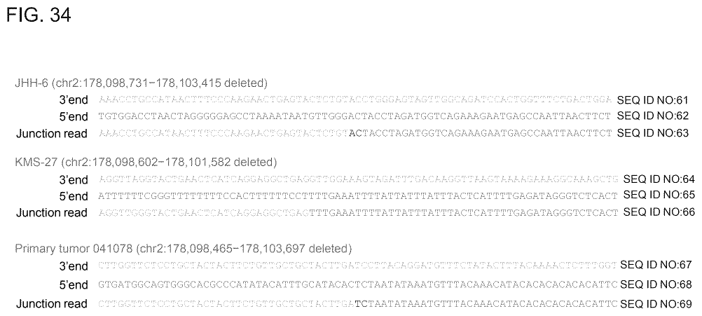

Analysis of the structure of the three deletions identified by WGS showed that breakpoints were distinct, but in each case genomic regions flanking the deletions showed 2-6 nucleotides with sequence homology (FIG. 34). The DNA sequences of the 3′ end, 5′ end, and junction read of JHH-6 cells are provided by SEQ ID NOs: 61-63, respectively. The DNA sequences of the 3′ end, 5′ end, and junction read of KMS-27 cells are provided by SEQ ID NOs: 64-66, respectively. The DNA sequences of the 3′ end, 5′ end, and junction read of primary tumor cells are provided by SEQ ID NOs: 67-69, respectively.

NRF2 often shows genomic amplification in addition to point mutations. Interestingly, while the intensity of the NRF2 deletion product in KMS-27 cells by RT-PCR analysis appeared similar to wild-type NRF2, it seemed to be more abundant in JHH-6 cells (FIG. 13). This was also reflected in WGS read counts, which suggested a higher abundance of the deleted form compared to the wild-type allele (FIG. 27B). These results are consistent with the observation that JHH-6 cells carry five copies of the NRF2 gene locus by SNP array, whereas KMS-27 cells carry two copies. Amplification of NRF2 is reasonably frequent in the TCGA samples analyzed, including squamous (4.5%) and adenomatous (2.6%) NSCLC, HNSC (12.2%), and liver cancers (3.6%), and represents a mechanism to increase NRF2 transcriptional output. In the case of JHH-6 cells, these data suggest that the deleted allele has been preferentially amplified, providing an additional mechanism to boost NRF2 signaling in this cell line. However, preferential amplification of the truncated/spliced allele was not observed in the primary tumors, suggesting that exon 2 or 2+3 deletion alone can provide sufficient NRF2 activity for clonal selection.

Deletion of exon 2 provides an elegant mechanism to increase NRF2 activity by removing the interaction site with KEAP1, while keeping the remainder of the gene functionally intact for DNA binding and transcriptional activation functions. Indeed, our biochemical analyses confirmed the almost complete loss of KEAP1 binding and resulting stabilization of NRF2 when exon 2 is deleted (FIGS. 23 and 24). When considering NRF2 point mutations found in tumors, mutations surrounding the ETGE high-affinity binding site result in complete loss of KEAP1 interaction, whereas mutations in the lower affinity DLG motif vary in their ability to disrupt the NRF2/KEAP1 complex (Fukutomi et al. Mol Cell Biol. 34 (5): 832-846, 2014; Shibata et al. Proc. Natl. Acad. Sci. USA. 105 (36): 13568-13573, 2008). However, even point mutations that do not disrupt the complex change the nature of the interaction such as to prevent KEAP1-mediated ubiquitination of NRF2 (Shibata et al. Proc. Natl. Acad. Sci. USA. 105 (36): 13568-13573, 2008). While the interaction with KEAP1 is similarly abolished in the case of deletion of both exon 2 and 3, exon 3 contains the Neh4 domain that has been previously implicated in transcriptional activation by NRF2 through binding to CREB (CAMP Responsive Element Binding protein) Binding Protein (CBP) (Katoh et al. Genes Cells. 6 (10): 857-868, 2001). Neh4 (contained in exon 3) and Neh5 (contained in exon 4) were shown to act synergistically in recruiting CBP. Consistent with this, a decreased ability of Δe2+3 NRF2 to induce some NRF2 target genes compared to Δe2 NRF2 or tumor-associated point mutations in NRF2 was observed (FIGS. 25 and 26).

Deletions found in human tumors that remove the interaction domain with E3 ligases have also been observed in other genes. For example, 7 out of 222 colorectal tumors showed small genomic deletions (234-677 bp) surrounding exon 3 of β-catenin (Iwao et al. Cancer Res. 58 (5): 1021-1026, 1998) that removes the interaction site for its E3 ligase β-TRCP (Hart et al. Curr. Biol. 9 (4): 207-210, 1999). Similarly, the majority of TMPRSS-ERG fusion proteins found in prostate cancer encode truncated versions of ERG that render them resistant to ubiquitination and degradation mediated by SPOP (An et al. Mol. Cell. 59 (6): 904-916, 2015).

In addition, mutations resulting in MET exon 14 skipping remove amino acid residue Y1003, which is required for Cbl recruitment and subsequent ubiquitination and down-regulation. Therefore, small intragenic deletions represent effective mechanisms for nascent oncogenes to escape normal degradation during tumor initiation and evolution.

### Example 8

### NRF2 knockdown in mutant KEAP1 cells

This example provides a characterization of the effects of KEAP1 mutations on the requirement for NRF2 activity under different growth environments and shows that NRF2 activity is essential for growth in anchorage independent conditions.

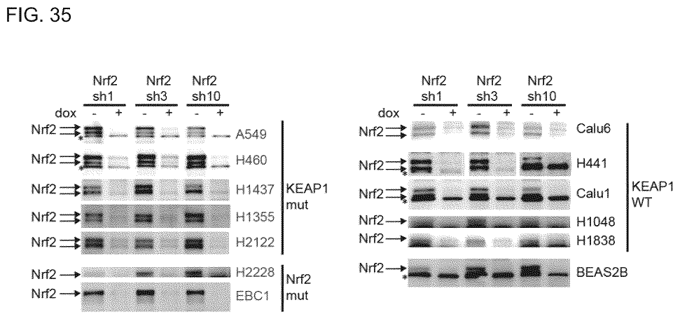

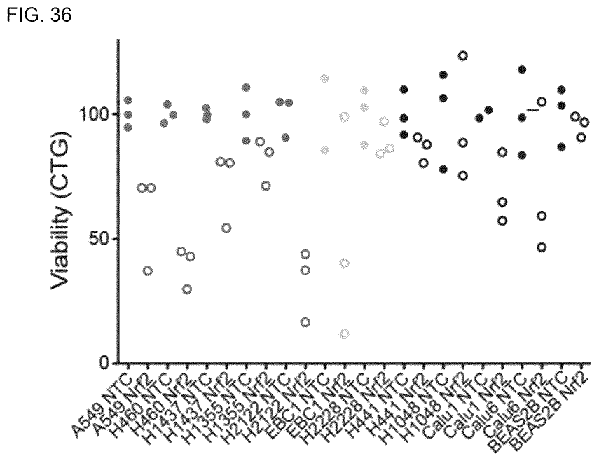

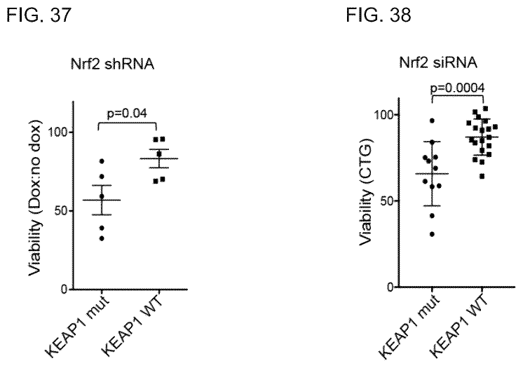

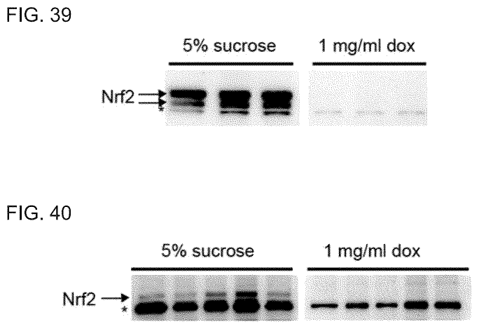

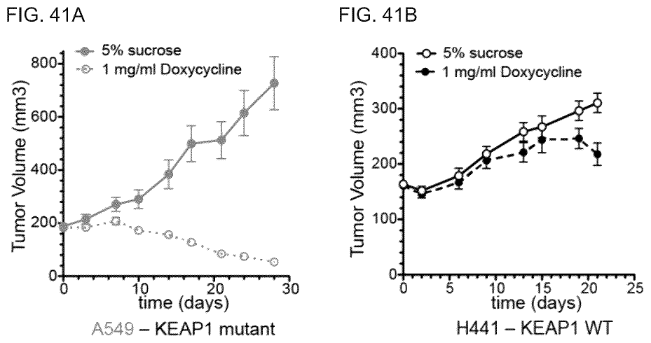

The consequences of NRF2 inhibition across wild-type and mutant KEAP1 and NRF2 cell lines were examined. Stable cell lines expressing three independent NRF2 shRNAs under the control of doxycycline, as well as three independent non-targeting controls (NTCs) were established. These NRF2 shRNAs were effective at reducing NRF2 protein levels in five KEAP1 mutant cell lines, two NRF2 mutant cell lines, and five wild-type NSCLC cell lines, as well as in immortalized but non-transformed lung epithelial BEAS2B cells (FIG. 35). Upon doxycycline addition, viability of most cell lines was decreased to varying extents, with the KEAP1 mutant cell lines generally exhibiting significantly greater decreases (FIGS. 36 and 37). Knockdown of NRF2 by siRNA in a larger panel of NSCLC cell lines confirmed a genotype-dependent effect on cell viability (FIG. 38). The consequence of NRF2 knockdown in tumor xenografts was characterized. The KEAP1 mutant A549 cell line and the KEAP1 wild-type H441 cell lines expressing dox-inducible NRF2 shRNAs were implanted into the flanks of female SCID mice. NRF2 was effectively knocked down in doxycycline treated mice in both tumors (FIGS. 39 and 40). NRF2 knockdown in the KEAP1 mutant A549 cell line had a dramatic effect on tumor growth, resulting in complete tumor regression in 5 out of 10 tumors (FIG. 41A). In contrast, the effect on KEAP1 wild-type H441 growth was more modest, resulting in a 37% reduction in tumor growth with all animals displaying maintained tumor burden (FIG. 41B).

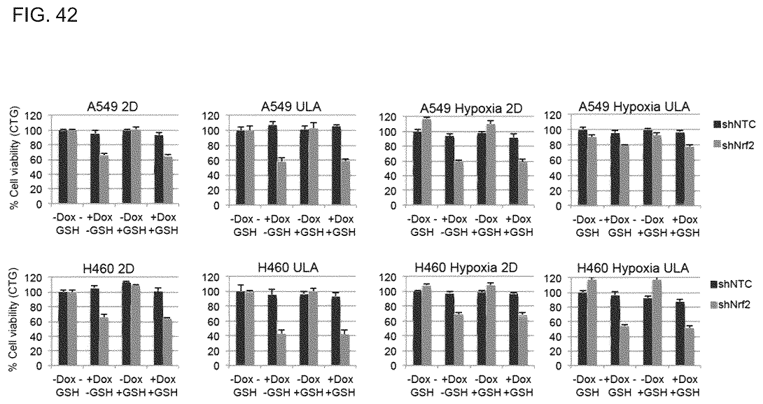

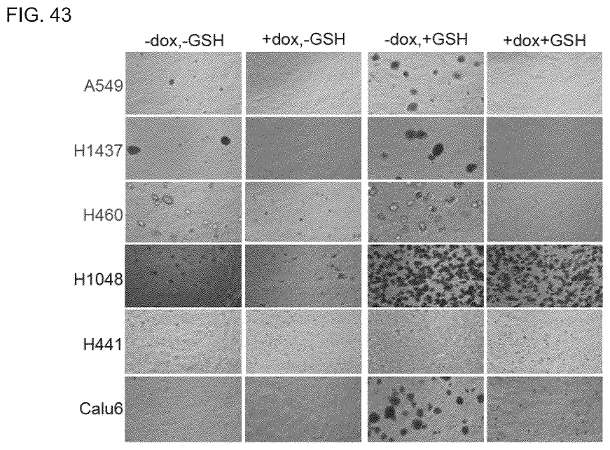

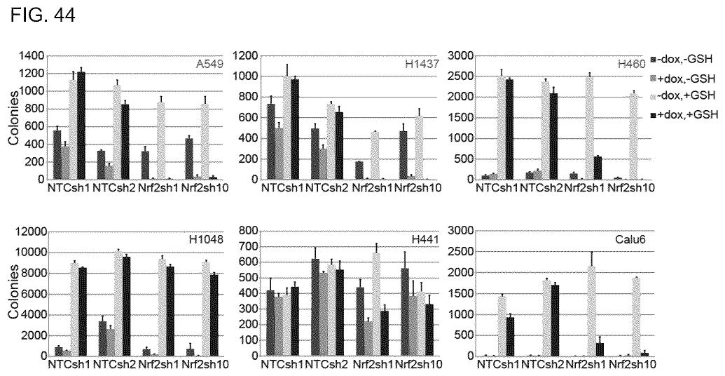

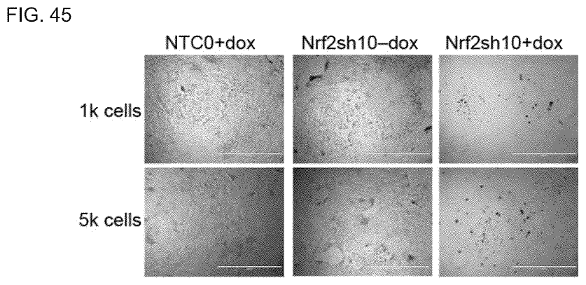

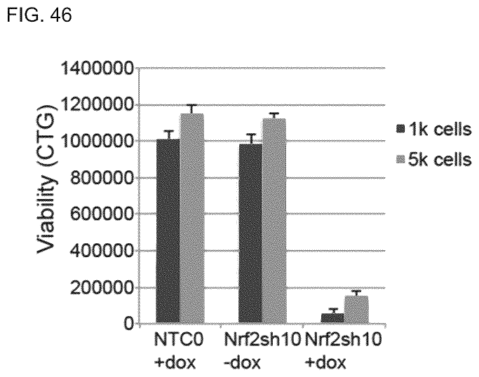

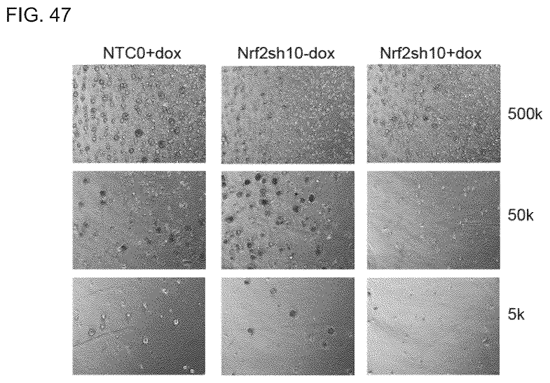

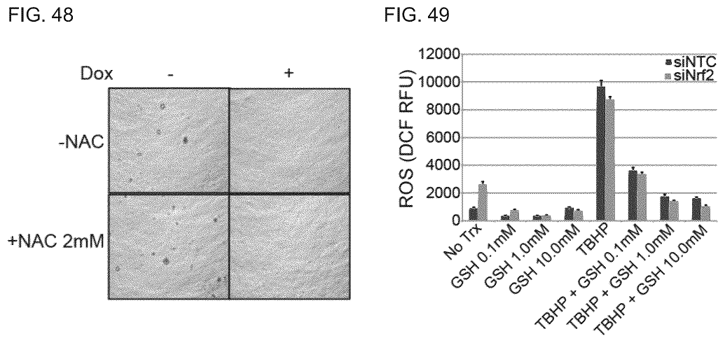

To understand the differential effects between NRF2 knockdown on tumor propagation in xenografts versus 2D growth on plastic, several additional cell culture environments were tested. NRF2 knockdown in cells grown in low adherence plates and/or low oxygen (0.5%) showed similar consequences to cells grown on plastic (FIG. 42). In contrast, the growth of KEAP1 mutant cell lines was severely compromised when cultured in soft agar (FIGS. 43 and 44), on micropatterned plastic films (FIGS. 45 and 46), or in methylcellulose (FIG. 47). Growth in soft agar was used to characterize the consequences of NRF2 knockdown in more detail. While knockdown of NRF2 completely abolished colony formation in three KEAP1 mutant cell lines, it had almost no effect in H1048 and H441, two wild-type KEAP1 NSCLC cell lines (FIGS. 43 and 44). The role of the glutathione pathway in the response to NRF2 knockdown was assessed, as this pathway has been shown to mediate survival properties facilitated by high NRF2 activity. While addition of reduced glutathione generally increased the ability of all tested cell lines to form colonies in soft agar, it was unable to rescue the consequences of NRF2 knockdown (FIGS. 43 and 44). Similar negative results were seen with N-acetyl cysteine (NAC; FIG. 48). Exogenous glutathione was able to enter cells and reduce reactive oxygen (ROS) levels, as measured by dichlorofluorescein staining (FIG. 49). Thus, the requirement for NRF2 activity is surprisingly independent of the glutathione synthesis pathway.

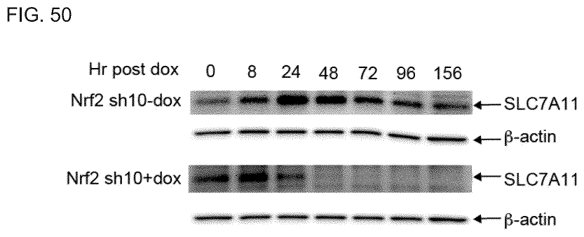

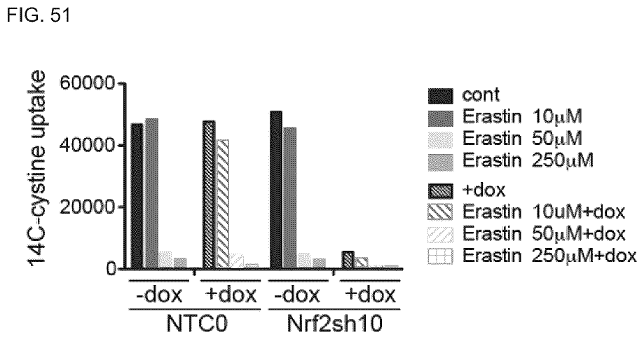

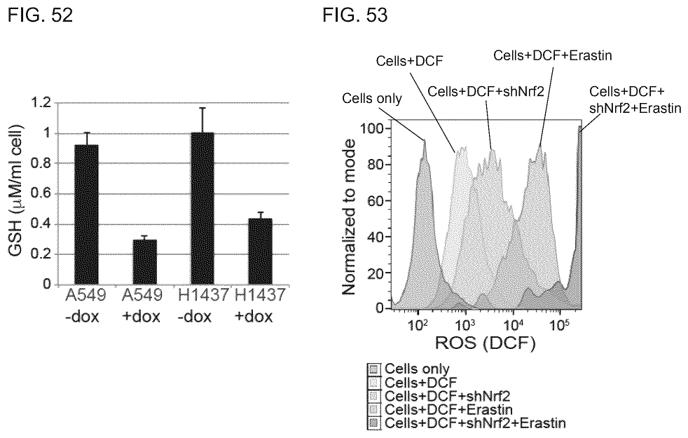

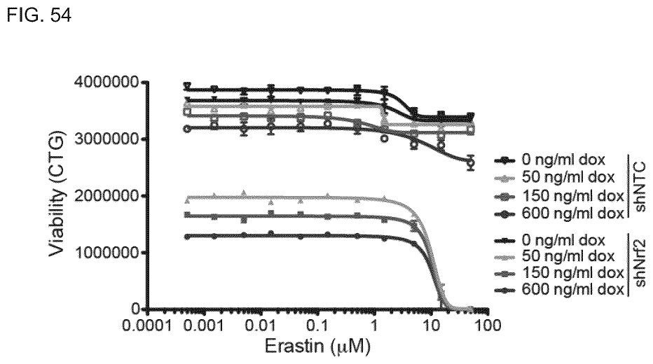

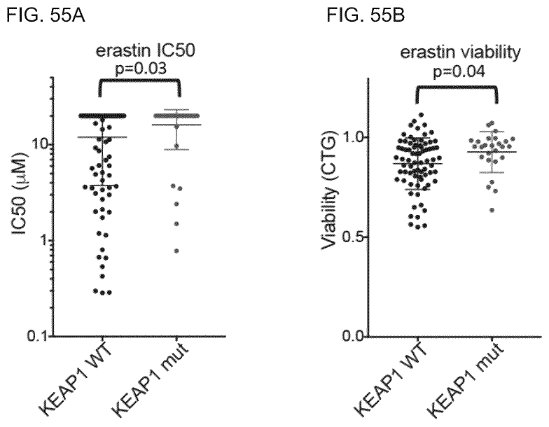

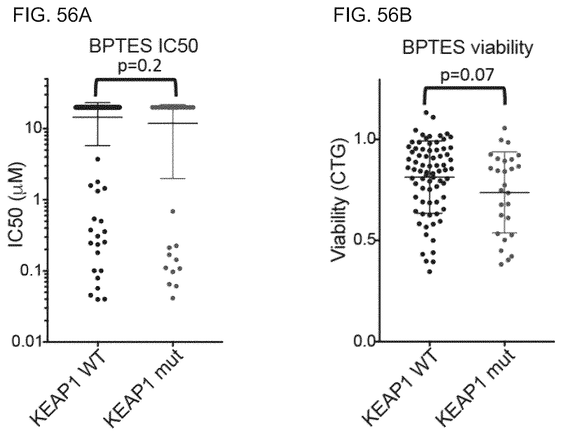

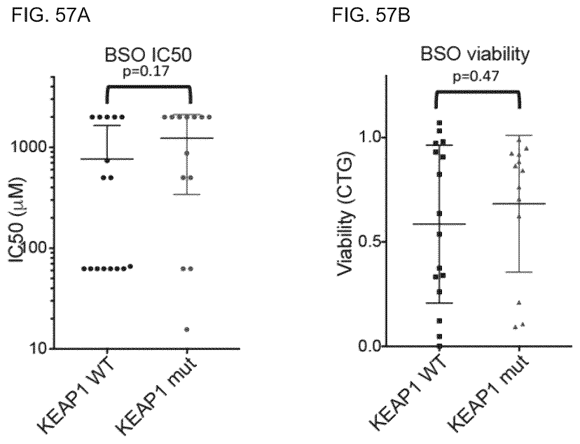

To further explore the effects of the glutathione pathway in NRF2 responses, the expression and activity of the xCT glutamate/cysteine antiporter, one of the rate limiting steps in glutathione synthesis, was monitored. SLC7A11 expression was reduced following NRF2 knockdown (FIG. 50), causing a decrease in cystine uptake (FIG. 51) associated with reduced glutathione (FIG. 52). NRF2 knockdown also caused a large increase in ROS levels (FIG. 53). To determine whether inhibition of SLC7A11 expression and cystine uptake contributed to decreased viability following NRF2 knockdown, xCT function was initiated using erastin, which inhibited cystine uptake (FIG. 51) and increased oxidative stress (FIG. 53). However, this was not sufficient to decrease the viability of the KEAP1 mutant cell line A549 (FIG. 54) or most other KEAP1 mutant cell lines (FIG. 55). The combination of erastin and NRF2 knockdown, however, did result in a dramatic decrease in viability (FIG. 54). Similarly, the glutathione synthase inhibitor buthionine sylphoximine (BSO) or the glutaminase inhibitor BPTES also did not display preferential toxicity for KEAP1 mutant cell lines (FIGS. 56 and 57). These results indicate that supplementation with glutathione was not sufficient to rescue lethality induced by NRF2 knockdown, nor was depletion of glutathione sufficient to kill KEAP1 mutant cell lines.

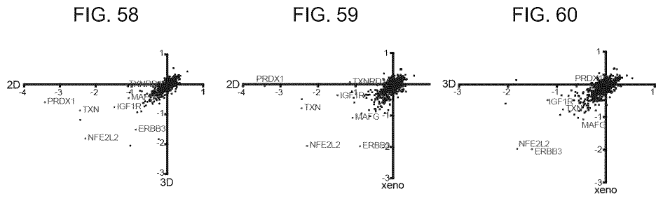

In order to understand which pathways were activated as a consequence of NRF2 activation or KEAP1 loss, a CRISPR screen was performed using a library of genes that were decreased upon NRF2 knockdown in A549 cells and/or elevated in a panel of KEAP1 mutant NSCLC cell lines. As distinct consequences were observed following NRF2 knockdown in 2D, 3D, and xenograft growth conditions, the screen was performed under all three environments to determine whether discrete dependencies could be identified. At 15-day time point for all three conditions, all three screens performed similarly, with gRNAs representing only a small number of genes showing significant drop-out (FIGS. 58-60). NFE2L2 and its binding partner, MAFG, were among the most significant genes, showing that the screen performed as expected. The pentose phosphate pathway genes PGD, G6PD and TKT, known NRF2 target genes, also showed strong drop-out. Other strong hits in the screen were two growth factor receptor genes, IGF1R and ERBB3, and genes encoding three components of a redox signaling relay, PRDX1, TXN, and TXNRD1.

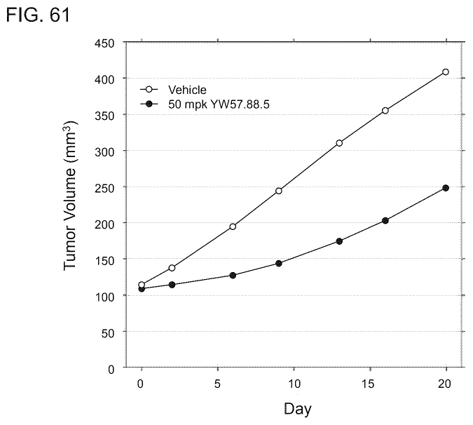

Expression of ErbB3 was decreased following NRF2 knockdown in A549 cells (FIGS. 58-60). Treatment with YW57.88.5 in a tumor xenograft model indicated that ErbB3 was required for A549 proliferation (FIG. 61).

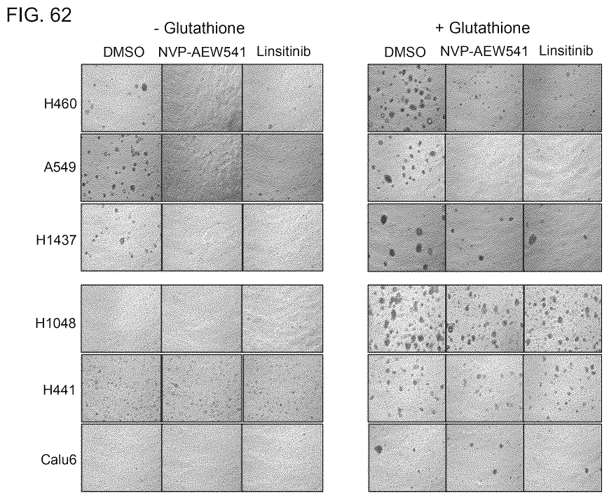

Expression of IGF1R was greater in KEAP1 mutant NSCLC cells relative to KEAP1 wild-type NSCLC cell lines. To test the effect of IGF1R inhibition on KEAP1 mutant and KEAP1 wild-type cells, cell lines were treated with linsitinib, a potent and selective IGF1R small molecule inhibitor. Linsitinib showed little effect on proliferation when tested in three wild-type and three mutant KEAP1 NSCLC cell lines. However, this compound was very potent at inhibiting colony growth of A549 cells in soft agar, having an IC₅₀ of about 20 nM. Moreover, when tested against a large panel of NSCLC cell lines, there appeared to be a selective growth inhibition in soft agar of this compound in KEAP1 mutant cell lines. A similar selective effect on KEAP1 mutant cell lines when grown under anchorage independent conditions was also seen with an independent IGF1R inhibitor NVP-AEW541 (FIG. 62).

Thus, growth factors signaling through IGF1R and ErbB3 are significant mediators of the growth of KEAP1 mutant cells.

### Other Embodiments

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1-40. (canceled)

41. A method of treating a subject having a cancer, the method comprising:
(a) determining the expression levels of the following 27 genes: AKR1B10, AKR1C2, SRXN1, OSGIN1, FECH, GCLM, TRIM16, ME1, KYNU, CABYR, SLC7A11, TRIM16L, AKR1C4, CYP4F11, RSPO3, ABCC2, AKR1B15, NR0B1, UGDH, TXNRD1, GSR, AKR1C3, TALDO1, PGD, TXN, NQO1, and FTL in a sample obtained from the subject; and comparing the expression level of each of the 27 genes to a reference expression level of each of the 27 genes, wherein an increase in the expression level of each of the 27 genes in the sample relative to the respective reference expression level of each of the 27 genes identifies the subject as likely to respond to a NRF2 pathway antagonist; and
(b) administering to the subject a therapeutically effective amount of a NRF2 pathway antagonist.

42-47. (canceled)

48. The method of claim 41, wherein (a) the expression level of each of the 27 genes in the sample is an average expression level of each of the 27 the genes of the sample; and
(b) the reference expression level of each of the 27 genes is an average expression level of each of the 27 genes in a population of subjects having the cancer.

49-53. (canceled)

54. The method of claim 41, wherein the expression level is an mRNA expression level.

55. The method of claim 54, wherein the mRNA expression level is determined by PCR, RT-PCR, RNA-seq, gene expression profiling, serial analysis of gene expression, or microarray analysis.

56. (canceled)

57. The method of claim 41, wherein the expression level is a protein expression level.

58. The method of claim 57, wherein the protein expression is determined by western blot, immunohistochemistry, or mass spectrometry.

59-76. (canceled)

77. The method of claim 41, wherein the NRF2 pathway antagonist is selected from the group consisting of a CREB antagonist, a CREB Binding Protein (CBP) antagonist, a Maf antagonist, an activating transcription factor 4 (ATF4) antagonist, a protein kinase C (PKC) antagonist, a Jun antagonist, a glucocorticoid receptor antagonist, a UbcM2 antagonist, a HACE1 antagonist, a c-Myc agonist, a SUMO agonist, a KEAP1 agonist, a CUL3 agonist, or a retinoic acid receptor α (RARα) agonist.

78. The method of claim 41, wherein the sample obtained from the subject is a tumor sample.

79. The method of claim 78, wherein the tumor sample is from a biopsy.

80. (canceled)

81. The method of claim 41, wherein the subject has a lung cancer or a head and neck cancer.

82. The method of claim 81, wherein the lung cancer is a non-small cell lung cancer (NSCLC).

83. The method of claim 82, wherein the NSCLC is a squamous NSCLC.

84. The method of claim 81, wherein the head and neck cancer is a squamous head and neck cancer.
